(19) United States
(12) Patent Application Publication
(10) Pub. No.: US 2021/0198676 A1
(43) Pub. Date: Jul. 1, 2021

(54) TEMPERATURE CONTROLLED DNA POLYMERASE INHIBITORS

(71) Applicant: SWIFT BIOSCIENCES, INC., Ann Arbor, MI (US)

(72) Inventors: Vladimir MAKAROV, Ann Arbor, MI (US); Jordan ROSEFIGURA, Ann Arbor, MI (US); Ashley M. WOOD, Ann Arbor, MI (US)

(21) Appl. No.: 17/138,996

(22) Filed: Dec. 31, 2020

Related U.S. Application Data
(63) Continuation of application No. PCT/US2019/040367, filed on Jul. 2, 2019.
(60) Provisional application No. 62/693,265, filed on Jul. 2, 2018.

(51) Int. Cl. C12N 15/113; C12Q 1/686

(52) U.S. Cl. CPC C12N 15/1137; C12Q 1/686; C12N 2310/335; C12N 2310/321; C12N 2310/14

(57) ABSTRACT

The present disclosure provides polynucleotide-based inhibitors for reversible activation of DNA polymerases. Use of lower Tm polynucleotide-based inhibitors allow PCR reaction assembly at room temperature while activating polymerase at higher PCR primer annealing temperatures, where the reversible nature of the inhibition additionally improves priming specificity during each PCR cycle. Additionally, temperature controlled inactivation of polymerase activity after PCR or other polymerase based enzymatic incubation eliminates a purification step when needed for compatibility with subsequent enzymatic incubations. For this application, the Tₘ of the polynucleotide-based inhibitor is higher than the desired reaction conditions of the subsequent enzymatic incubation.

### CROSS-REFERENCE TO RELATED APPLICATIONS

The present application is a continuation of International Application No. PCT/US2019/040367, filed Jul. 2, 2019, published as WO 2020/010124, which claims priority to U.S. Provisional Application No. 62/693,265, filed Jul. 2, 2018, the entirety of each of which is incorporated herein by reference.

### SEQUENCE LISTING

The instant application contains a Sequence Listing which has been submitted electronically in ASCII format and is hereby incorporated by reference in its entirety. Said ASCII copy, created on Jul. 1, 2019, is named 18-21019-WO_SL.txt. and is 134,472 bytes in size.

### BACKGROUND

Priming specificity is a key factor when performing either single or multiplexed PCR. With the advent of targeted next generation sequencing (NGS), there is a significant need for highly multiplexed PCR to simultaneously amplify hundreds to thousands of genomic target regions in a single tube. Although advances in primer design and reaction chemistries have largely overcome the formation of PCR artifacts including primer dimer formation and off-target amplification, certain target loci present challenging sequence content for assay design and additional measures to improve specificity are needed. These include target regions of lower base composition complexity and target regions with related pseudogenes or gene homologues of similar sequence. Reduction of such artifacts is critical for targeted next generation sequencing panels as the presence of primer dimers and off-target amplification artifacts increase the cost of sequencing when present in the NGS library as both generate sequenceable products. The addition of the disclosed temperature controlled polymerase inhibitors demonstrate an improvement to both on-target amplification in a highly multiplexed PCR assay as well as a reduction in primer dimer formation.

Additionally, the disclosed polynucleotide-based inhibitors can be used to further simplify NGS library preparation and other workflows by eliminating the need for a purification step following the use of a polymerase when needed for compatibility with subsequent enzymatic steps. For example, inactivation of polymerases used for DNA end repair prior to NGS adapter ligation allows one to eliminate a bead-based purification step. Similarly, if adapter ligation is performed following multiplexed PCR, polymerase activity can be inhibited for the subsequent DNA ligase-mediated NGS adapter ligation step.

Although antibody hotstart polymerase inhibitors are widely used, the advantage of the disclosed temperature controlled polymerase inhibitors is that their activity is reversible, similar to aptamer-based hotstart polymerase inhibitors. An advantage of the present polymerase inhibitors over aptamer-based inhibitors is in the flexibility and ease of design. For polymerase specific inhibition, different replication blocking modifications are used, and by altering the Tm of the partially double-stranded duplex portion, inhibition of polymerase activity can simply be adjusted for reaction temperatures from 16° C. to >75° C. With the disclosed inhibitors, it is possible to inhibit one polymerase type while another polymerase type remains active. A second advantage over some aptamers that require a stem-loop structure for inhibition is that the disclosed inhibitors are comprised of two independent oligonucleotides where annealing and denaturation occurs more cooperatively in a narrower temperature range. This is due to the bimolecular vs. intramolecular sequence complementarity, thereby making them easier to fine-tune to specific reaction conditions.

### SUMMARY

The present disclosure provides compositions and methods for using polymerase inhibitors in PCR and subsequent enzymatic processing steps.

The polymerase inhibitors of the present disclosure include a partially double-stranded polynucleotide duplex with at least one 5′ overhang (also referred to herein as a “single-stranded region”) as shown in FIG. 1. The polymerase inhibitors comprise at least one corresponding recessed 3′ end at the terminus of the partially double-stranded DNA duplex, which represents a natural intermediate of DNA replication and has an increased affinity for DNA polymerase binding. In addition, the 5′ overhang can include a replication blocking sequence or the 3′ terminus (recessed 3′ end) comprises an extension blocking group to prevent polymerase extension. In this regard, such partially double-stranded polynucleotide duplexes can be used as a sink for DNA polymerase binding, which sequesters and prevents polymerase activity because the inclusion of the replication blocking sequence or the extension blocking group maintains the 5′ overhang which can persist in the presence of DNA polymerase activity. Allowing extension of the 3′ recessed end to replicate across the 5′ overhang would render the polynucleotide inert and would no longer serve as a sink for polymerase binding and reversible inactivation of polymerase activity. In terms of inhibiting a polymerase, this can include both polymerase activity as well as exonuclease activity inherent to these enzymes. Additionally, polymerase inhibitors comprising two or more 5′ overhangs, one at each terminus can also be used in order to increase the likelihood of polymerase binding.

In some embodiments, a polymerase inhibitor can include a synthetic nucleic acid molecule which includes a first oligonucleotide comprising a first complementary region and a second oligonucleotide comprising a second complementary region and a first single-stranded region positioned 5′ to the second complementary region, where the first complementary region is sufficiently complementary to the second complementary region to form a double-stranded region at a temperature below a melting temperature of the polymerase inhibitor and the first single-stranded region includes a first replication blocking sequence or the first oligonucleotide further comprises a first extension blocking group.

In some embodiments, a polymerase inhibitor of the present disclosure can include a synthetic nucleic acid molecule which can include a first oligonucleotide and a second oligonucleotide, where the first oligonucleotide can include a first complementary region and the second oligonucleotide can include a second complementary region and a first single-stranded region positioned 5′ to the second complementary region, where the first complementary region and the second complementary region are sufficiently complementary to form a double-stranded region at a temperature below a melting temperature of the polymerase inhibitor, where the first oligonucleotide also includes a second single-stranded region positioned 5′ to the first complementary region, where the first single-stranded region can include a first replication blocking sequence or the first oligonucleotide can include a first extension blocking group at a 3′ end of the first oligonucleotide, and where the second single-stranded region can include a second replication blocking sequence or the second oligonucleotide can include a second extension blocking group at a 3′ end of the second oligonucleotide.

In some embodiments, a kit of the present disclosure can include a polymerase inhibitor of the present disclosure and a polymerase.

In some embodiments, a method for performing PCR includes the steps of (i) combining a polymerase inhibitor of the present disclosure with a thermostable polymerase, deoxynucleotides, a substrate polynucleotide, and at least one primer pair comprising a forward primer and a reverse primer sufficient to amplify a target portion of the substrate polynucleotide to form a first reaction mixture under conditions sufficient for the first complementary region and secondary complementary region to form a double-stranded region, and (ii) incubating the first reaction mixture under conditions sufficient to (a) dissociate the first oligonucleotide and the second oligonucleotide of the polymerase inhibitor, (b) allow the forward and reverse primers to anneal to the substrate polynucleotide, and (c) allow the thermostable polymerase to extend the forward and reverse primers to yield PCR amplicons.

In some embodiments, a method for inhibiting polymerase in a PCR reaction product can include the steps of (i) adding a polymerase inhibitor of the present disclosure to a reaction product that includes a thermostable polymerase and PCR amplicons and (ii) enzymatically processing the PCR amplicons at an enzymatic processing temperature.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the disclosure is not limited to specific methods and instrumentalities disclosed herein.

FIG. 1 depicts a schematic of polymerase inhibitor activity. The inhibitor is depicted as two oligonucleotides (grey lines) that are partially double-stranded when annealed at low temperature, where the replication blocking modification is depicted (lighter grey portion) and bound, inactive polymerase is depicted by ovals. As the temperature is increased above the Tm of the duplex, the oligonucleotides and polymerase dissociate and the polymerase becomes active.

FIG. 2 provides examples of lower melting temperature (Tₘ) inhibitors for reversible inhibition of high fidelity DNA polymerases for PCR. The 4 riboU bases are a replication blocking modification, and the duplex is designed using a low complexity sequence to facilitate rapid annealing. When the inhibitor molecules are added at a molar excess to both the polymerase molecules present as well as the primed active substrate molecules, binding and inhibition of high fidelity DNA polymerases occurs during PCR reaction assembly at room temperature but at elevated temperature above the duplex Tₘ, oligonucleotide denaturation leads to release and activation of polymerase activity (at temperatures above 50° C. for these example polynucleotides). FIG. 2 discloses SEQ ID NOS 556-559, respectively, in order of appearance.

FIG. 3 provides an example of a polymerase inhibitor that will inhibit polymerase at low temperature or high temperature due to the higher Tₘ of the duplex portion. As an alternative to a bead-based purification, this inhibitor can be added at a molar excess to both the polymerase molecules as well as subsequent oligonucleotide substrate molecules following PCR or other polymerization reactions in order to inactivate polymerase when needed for compatibility with downstream enzymatic incubations. Four riboU bases are used as a replication block for high fidelity polymerase, but different modifications can be incorporated for inhibition of Taq (e.g. 3 riboG bases) or any polymerase (e.g. stable a basic site, carbon spacer). The Tₘ of this inhibitor duplex should be higher than the temperature needed for subsequent enzymatic incubations. FIG. 3 discloses SEQ ID NOS 560-561, respectively, in order of appearance.

FIG. 4 provides an example of DNA polymerase inhibitors with different duplex melting temperature to be used as reversible inhibitors for room temperature PCR reaction assembly (low Tm inhibitors) or as inhibitors following PCR or other polymerization reactions when needed for compatibility with downstream enzymatic incubations (high Tₘ inhibitors). The 4 riboU replication block is specific for high fidelity polymerases, whereas the 3 riboG replication block is specific for Taq polymerase. FIG. 4 discloses SEQ ID NOS 562-577, respectively, in order of appearance.

FIG. 5 provides next generation sequencing metrics for Example 1.

FIG. 6 provides next generation sequencing metrics for Example 2.

FIG. 7 provides next generation sequencing metrics for Example 3.

### DETAILED DESCRIPTION

While compositions and methods are described herein by way of examples and embodiments, those skilled in the art recognize the compositions and methods are not limited to the embodiments or drawings described. It should be understood that the drawings and description are not intended to be limited to the particular form disclosed. Rather, the intention is to cover all modifications, equivalents and alternatives falling within the spirit and scope of the appended claims and description. Any headings used herein are for organization purposes only and are not meant to limit the scope of the description of the claims. As used herein, the words “may” and “can” are used in the permissive sense (i.e., meaning having the potential to) rather than the mandatory sense (i.e. meaning must). Similarly, the words “include,” “including,” and “includes” mean including, but not limited to. The present disclosure describes particular embodiments and with reference to certain drawings, but the subject matter is not limited thereto.

The present disclosure will provide description to the accompanying drawings, in which some, but not all embodiments of the subject matter of the disclosure are shown. Indeed, the subject matter may be embodied in many different forms and should not be construed as limited to the embodiments set forth herein, rather, these embodiments are provided so that this disclosure satisfies all the legal requirements.

Definitions

Certain terminology is used in the following description for convenience only and is not limiting. Unless specifically set forth herein, the terms “a,” “an,” and “the” are not limited to one element, but instead should be read consistent with the meaning of “one or more,” “at least one,” and “one or more than one.” As used herein “another” means at least a second or more. The terminology includes the words noted above, derivatives thereof and words of similar import.

The use of the term “or” in the claims is used to mean “and/or” unless explicitly indicated to refer to alternatives only or the alternatives are mutually exclusive.

Use of the term “about,” when used with a numerical value, is intended to include +/−10%. For example, if a number of nucleotides is identified as about 200, this would include 180 to 200 (plus or minus 10%).

As used herein, the term “synthetic,” with respect to a nucleic molecule refers to a nucleic acid molecule produced by in vitro chemical and/or enzymatic synthesis.

Unless otherwise defined, all technical and scientific terms used herein have the same meaning as commonly understood by one of ordinary skill in the art.

The present disclosure generally relates to compositions and methods for using polymerase inhibitors in PCR and subsequent enzymatic processing steps.

Polymerase Inhibitors

Generally, the polymerase inhibitors of the present disclosure include a partially double-stranded polynucleotide duplex with at least one 5′ overhang, where the 5′ overhang includes a replication blocking sequence or the recessed 3′ end includes an extension blocking group. The polymerase inhibitors of the present disclosure can include two 5′ overhangs. In such cases, the second 5′ overhang can include a replication blocking sequence or the second recessed 3′ end can include an extension blocking group.

In some embodiments, a polymerase inhibitor of the present disclosure can include a synthetic nucleic acid molecule which can include a first oligonucleotide and a second oligonucleotide, where the first oligonucleotide can include a first complementary region and the second oligonucleotide can include a second complementary region and a first single-stranded region positioned 5′ to the second complementary region, where the first complementary region and the second complementary region are sufficiently complementary to form a double-stranded region at a temperature below a melting temperature of the polymerase inhibitor, and where the first single-stranded region can include a first replication blocking sequence or the first oligonucleotide can include a first extension blocking group at a 3′ end of the first oligonucleotide.

In some embodiments, a polymerase inhibitor of the present disclosure, a polymerase inhibitor of the present disclosure can include a synthetic nucleic acid molecule which can include a first oligonucleotide and a second oligonucleotide, where the first oligonucleotide can include a first complementary region and the second oligonucleotide can include a second complementary region and a first single-stranded region positioned 5′ to the second complementary region, where the first complementary region and the second complementary region are sufficiently complementary to form a double-stranded region at a temperature below a melting temperature of the polymerase inhibitor, where the first oligonucleotide also includes a second single-stranded region positioned 5′ to the first complementary region, where the first single-stranded region can include a first replication blocking sequence or the first oligonucleotide can include a first extension blocking group at a 3′ end of the first oligonucleotide, and where the second single-stranded region can include a second replication blocking sequence or the second oligonucleotide can include a second extension blocking group at a 3′ end of the second oligonucleotide.

In some embodiments, the first oligonucleotide and the second oligonucleotide are separate oligonucleotides, i.e. they are not part of the same polynucleotide. In some embodiments, the first oligonucleotide and the second oligonucleotide can be part of the same polynucleotide. For example, the first oligonucleotide and second oligonucleotide can be connected and form a stem loop structure. In some embodiments, the synthetic nucleic acid molecule can further comprise a connecting region that is positioned between a 5′ end of the first oligonucleotide and the 3′ end of the second oligonucleotide. In some embodiments, the connecting region can include deoxynucleotides, ribonucleotides, inosine bases or carbon or other spacers.

In some embodiments, the synthetic nucleic acid molecule can further include an affinity label. By way of example, but not limitation, the affinity label can be biotin or digoxygenin. By way of example, but not limitation, biotin can be added during synthesis using a biotin-label deoxynucleotide.

Complementary Regions

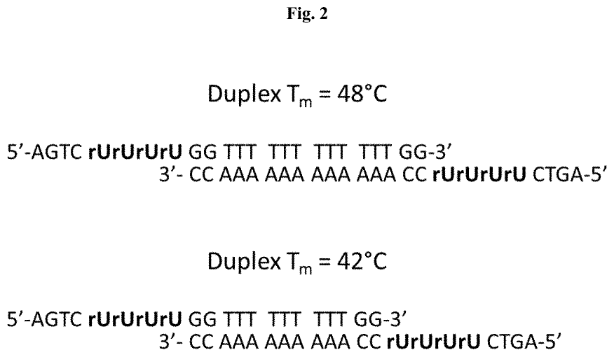

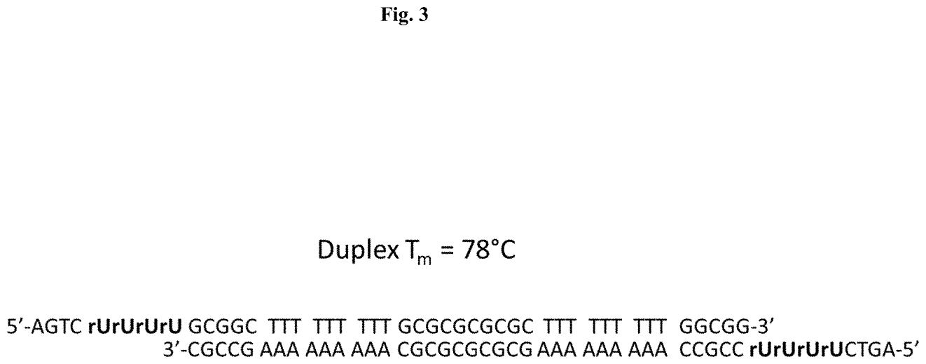

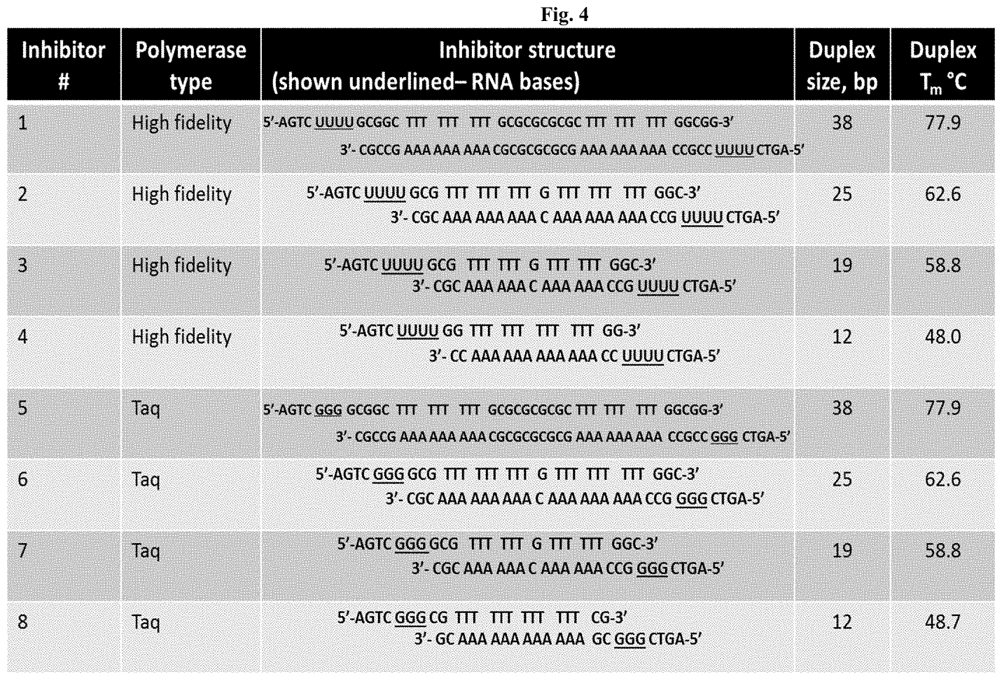

In any of the foregoing embodiments, the complementary regions of the first oligonucleotide and second oligonucleotide of the polymerase inhibitors of the present disclosure—the first complementary region and second complementary region, respectively—can be sufficiently complementary for the first oligonucleotide and the second oligonucleotide to form a double-stranded region at a temperature below a melting temperature. The sequences should not be self-complementary to avoid the formation of secondary structure when the first and second oligonucleotide are dissociated which can inhibit the formation and effectiveness of the inhibitor. In some embodiments, the double-stranded portion of the polymerase inhibitors of the present disclosure can include a low complexity sequence to increase the rate of annealing for duplex formation at permissive temperatures, such as those shown in FIGS. 2-4, e.g. the first complementary region can include the low complexity sequence which will impact the sequence of the second complementary region. In some embodiments, the low complexity sequence, can be selected from a homopolymeric sequence or a heteropolymeric sequence comprising a dinucleotide sequence. By way of example, but not limitation, homopolymeric sequences can include poly (dA), poly (dT), poly (dC), poly (dG), poly (dU), poly (rA), poly (U), poly (rC), and poly (rG). By way of example, but not limitation, a heteropolymeric sequence comprising a dinucleotide sequence can include: dA and rA bases, dT, dU and U bases, dC and rC bases, or dG and rG bases or random sequences of the following combinations: dG and dC; dA and dT; dG and dT; dG and dA; dA and dC; or dC and dT, or a mixture of ribonucleotide and deoxyribonucleotide. By way of further example, but not limitation, where low complexity sequence includes a homopolymer sequence, it can be flanked by a GC clamp, such as a T homopolymer that can anneal to an A homopolymer flanked by a GC clamp as shown in FIG. 2 where the T homopolymer is flanked by two G nucleotides at either end which can anneal to complementary C nucleotides on the opposite oligonucleotide. By way of further example, but not limitation, the homopolymer sequence can be flanked by a dinucleotide repeat portion of GC bases as shown in FIG. 3, which can be used for high annealing temperatures. In some embodiments, the first complementary region and the second complementary region can include deoxynucleotide bases and ribonucleotide bases. In some embodiments, the complementary regions can comprise a sequence including all 4 nucleotides. In some embodiments, the first complementary region and second complementary region do not comprise self-complementary sequences that can lead to secondary structure of the single-stranded inhibitor molecules.

In some embodiments, the complementary regions can include from about 6 to about 100 or more nucleotides. By way of example, but not limitation, the first complementary region and second complementary region can include 6, 7, 8, 9, 10, 11, 12, 13, 14, 15, 20, 25, 30, 35, 40, 45, 50, 55, 60, 65, 70, 75, 80, 85, 90, 95 or 100 nucleotides and any range or value therebetween. The length of the complementary regions, in addition to other factors such as the sequence of the first and second oligonucleotides, can impact the melting temperature of the polymerase inhibitor.

Single-Stranded Regions

Polymerase inhibitors of the present disclosure can include one or more single-stranded regions. These single-stranded regions can form 5′ overhangs which can further include replication blocking sequences. These 5′ overhangs can generate a natural substrate for polymerase extension due to the 3′ recessed end that is formed by the double-stranded region.

The single-stranded regions—the first single-stranded region and the second single-stranded region—can be of any length that is suitable for use as a polymerase inhibitor and to achieve the desired melting temperature. In some embodiments, the first single-stranded region is from 1 to about 100 nucleotides or more in length. By way of example, but not limitation, the first single-stranded region can include about 1, 2, 3, 4, 5, 6, 7, 8, 9, 10, 11, 12, 13, 14, 15, 20, 25, 30, 35, 40, 45, 50, 55, 60, 65, 70, 75, 85, 90, 95 or 100 nucleotides and any range or value therebetween. Similarly, in some embodiments, the second single-stranded region is from about 1 to about 100 nucleotides or more in length. By way of example, but not limitation, the second single-stranded region can include about 1, 2, 3, 4, 5, 6, 7, 8, 9, 10, 11, 12, 13, 14, 15, 20, 25, 30, 35, 40, 45, 50, 55, 60, 65, 70, 75, 85, 90, 95 or 100 nucleotides and any range or value therebetween.

Replication Blocking Sequences

The 5′ overhang(s) of the polymerase inhibitors of the present disclosure can further include a replication blocking sequence. A replication blocking sequence can be any suitable sequence that prevents extension by a polymerase such as, by way of example but not limitation, a general polymerase replication blocking sequence such as a stable abasic site or a carbon spacer. In some embodiments, the replication blocking sequence can be selected from the group consisting of a stable abasic site, a carbon spacer, at least one 2′-O methyl nucleotide, a deoxyuridine base, a deoxyinosine base, and from about 2 to about 50 consecutive nucleotide bases, or any combination thereof. In some embodiments, the replication blocking sequence can include 2, 3, 4, 5, 6, 7, 8, 9, 10, 11, 12, 13, 14, 15, 20, 25, 30, 35, 40, 45 or 50 consecutive ribonucleotides and any range of value therebetween. Certain replication blocking sequences can be specific to particular polymerases. By way of example but not limitation, a consecutive stretch of least two ribo(U) bases or at least 2 ribo(A) bases or a combination thereof can be used to block extension by high fidelity polymerases. Exemplary high fidelity polymerases include Kapa HiFi HotStart ReadyMix (Roche), Q5 DNA Polymerase (NEB), PrimeStar GXL Polymerase (Clontech), and Herculase DNA Polymerase (Agilent). By way of further example but not limitation, a consecutive stretch of at least two ribo(G) bases can be used to block extension by Taq polymerase. By way of further example but not limitation, a deoxyuridine base or deoxyinosine base can be used to block extension by a high fidelity proofreading polymerase, i.e. a high fidelity polymerase having 3′-5′ exonuclease activity, such as, by way of example, but not limitation Kapa HiFi HotStart ReadyMix (Roche).

In some embodiments, the replication blocking sequence is positioned relative to the 3′ recessed end such that it can prevent a partial extension reaction from the 3′ recessed end and preserve the 5′ overhang. By way of example but not limitation, the replication blocking sequence can be within 0, 1, 2, 3, 4, 5, 6, 7, 8, 9, or 10 nucleotides of the double-stranded region, i.e. the corresponding complementary region on the same oligonucleotide, or any range therebetween.

In some embodiments, the single-stranded region can include 5′ terminal deoxynucleotides positioned 5′ of the replication blocking sequence. By way of example, but not limitation, the single-stranded region can include 1, 2, 3, 4, 5, 6, 7, 8, 9, 10, 15, 20, 25, 30, 35, 40, 45, 50, 60, 70, 80, 90, 100 or more deoxynucleotides and any range therebetween. Exemplary terminal deoxynucleotide sequences are shown in FIGS. 2-4.

Extension Blocking Groups and Nuclease Resistant Modifications

In some embodiments, the first oligonucleotide or second oligonucleotide can further include an extension blocking group at a 3′ end of the first oligonucleotide or second oligonucleotide, respectively. As an alternative to a replication blocking sequence or in addition to one, an extension blocking group on the 3′ recessed end can prevent polymerase extension of the polymerase inhibitor. An extension blocking group is any group that, when included, at the 3′ end of the polymerase inhibitor with a complementary 5′ overhang, can prevent extension by a polymerase, while allowing annealing of the polymerase to the polymerase inhibitor when it is partially double-stranded. By way of example, but not limitation, an extension blocking group can be selected from the group consisting of a 3′ phosphate, a 3′ carbon or other spacer, a 3′ inverted dT, a 3′ amino modification and a 3′ dideoxynucleotide or any combination thereof.

Where an extension blocking group is included in a polymerase inhibitor of the present disclosure, a further nuclease resistant modification of the 3′ terminal end can be required to maintain the extension blocking group such as, by way of example but not limitation, when the polymerase is a high fidelity polymerase or polymerase with 3′ to 5′ exonuclease activity. By way of example, but not limitation, the nuclease resistant modification can be a 3′ terminal phosphorothioate linkage. In some embodiments, the nuclease resistant modification can be two or more phosphorothioate linkages. By way of example, but not limitation, the nuclease resistant modification can include 2, 3, 4 or more phosphorothioate linkages. In any of the foregoing embodiments, a polymerase inhibitor of the present disclosure can include both one or more replication blocking sequences and one or more extension blocking groups as well as nuclease resistant modifications.

Polymerases

In some embodiments, a polymerase inhibitor of the present disclosure can be bound to a polymerase. In some embodiments, a polymerase inhibitor of the present disclosure can be bound to one or more polymerases. In some embodiments, the polymerase inhibitor of the present disclosure is bound to two polymerases.

As noted, the types of replication blocking sequence can, in some instances, be specific to certain polymerases. The polymerase inhibitors of the present disclosure can generally be used to inhibit any polymerase, however, certain embodiments may be useful to inhibit specific polymerases such as Taq polymerase or a high fidelity polymerase.

Melting Temperatures

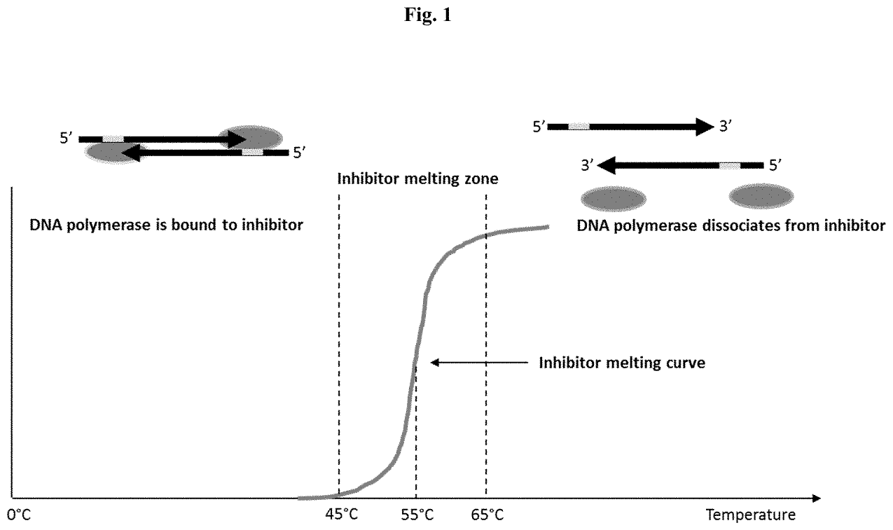

The melting temperature of the polymerase inhibitor can be calculated by known methods known to one of ordinary skill in the art. By way of example, but not limitation, commercial software such as that found on the IDT (idtdna.com), NEB (neb.com) and ThermoFisher (thermofisher.com) websites can be used to calculate the Tₘ of the polymerase inhibitor based on the sequences of the first oligonucleotide and the second oligonucleotide and on other parameters such as buffer, salt concentration and the like. In addition, in some instances, polymerase binding can alter the melting temperature by stabilizing the polymerase inhibitor. The melting temperature of the double-stranded portion of the inhibitor is important for regulating the switch to reversible polymerase inactivation. Incubation at temperatures below the melting temperature of the inhibitor duplex results in polymerase binding and inhibition of polymerase activity as shown in FIG. 1. Incubation at temperatures above the melting temperature of the partially dsDNA duplex results in denaturation, which also results in release of bound DNA polymerase and as a result, activation of polymerase activity, because DNA polymerases do not have an affinity for single-stranded polynucleotides.

In some embodiments, the melting temperature of the polymerase inhibitor is below 90° C., 89° C., 88° C., 87° C., 86° C., 85° C., 84° C., 83° C., 82° C., 81° C., 80° C., 79° C., 78° C., 77° C., 76° C., 75° C., 74° C., 73° C., 72° C., 71° C., 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C. or lower and any range or value therebetween. In some embodiments the melting temperature of the polymerase inhibitor is 90° C., 89° C., 88° C., 87° C., 86° C., 85° C., 84° C., 83° C., 82° C., 81° C., 80° C., 79° C., 78° C., 77° C., 76° C., 75° C., 74° C., 73° C., 72° C., 71° C., 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C. or lower and any range or value therebetween. The desired melting temperature of the polymerase inhibitor can depend on the application. By way of example, but not limitation, if a polymerase chain reaction (PCR) is to be performed, the melting temperature of the polymerase inhibitor can be below an annealing temperature or an extension temperature of the PCR. By adjusting the length, base composition, or both of the polymerase inhibitor, a desired melting temperature can be achieved. By way of further example, but not limitation, if the polymerase inhibitor is to be used to inhibit polymerase in a reaction such as an enzymatic processing reaction, the melting temperature can be above the enzymatic processing temperature. In any of the foregoing embodiments, the melting temperature refers to the temperature of the partially double-stranded polymerase inhibitor at which 50% of the partially double-stranded structure is denatured.

Kits

In some embodiments, a kit can include a polymerase inhibitor of the present disclosure. In some embodiments, a kit of the present disclosure can include a polymerase inhibitor of the present disclosure and a polymerase. In some embodiments, the polymerase is a thermostable polymerase. In some embodiments, a kit of the present disclosure can include a polymerase inhibitor of the present disclosure, a polymerase and at least one primer pair. In some embodiments, a kit of the present disclosure can include a polymerase inhibitor of the present disclosure, a polymer, at least one primer pair and deoxynucleotides. In any of the foregoing embodiments, a kit of the present disclosure can further comprise a reaction buffer. In any of the foregoing embodiments, a kit of the present disclosure can include a universal primer. In some embodiments, where a kit of the present disclosure includes a universal primer, it can also include a plurality of different target-specific primer pairs for amplifying a plurality of different loci of a nucleic acid substrate, wherein each of the plurality of target-specific primer pairs comprise a forward primer and a reverse primer, wherein the forward primer and the reverse primer comprise a 3′ complementary sequence that is complementary to a first sequence of the nucleic acid substrate and a second sequence of the nucleic acid sequence, respectively, wherein the first sequence and second sequence is different for each of the plurality of different target-specific primer pairs, wherein the forward and reverse primer of each of the plurality of different target-specific primer pairs further comprise a 5′ terminal sequence that is not complementary to the nucleic acid substrate, wherein the 5′ terminal sequence and the universal primer are complementary to a common sequence.

In any of the foregoing embodiments, the polymerase inhibitor of a kit can include a replication blocking sequence specific to the polymerase that is included in the kit. By way of example, but not limitation, where the kit includes Taq polymerase, the polymerase inhibitor can include at least one replication blocking sequence such as the first replication blocking sequence which includes a consecutive stretch of two or more ribo(G) bases. By way of further example, but not limitation, where the kit includes a high fidelity polymerase, the polymerase inhibitor can include at least one replication blocking sequence such as the first replication blocking sequence which includes a consecutive stretch of at least two ribo(U) bases or two ribo(A) bases.

In accordance with the present disclosure, a kit can include any of the components necessary to perform a reaction, such as a PCR reaction of enzymatic processing step, in addition to a polymerase inhibitor of the present disclosure.

In some embodiments, a kit of the present disclosure includes a polymerase inhibitor of the present disclosure and a ligase. In some embodiments, a kit of the present disclosure can include a polymerase inhibitor of the present disclosure, a ligase, and an adaptor polynucleotide.

In any of the foregoing embodiments where the polymerase inhibitor is not a single polynucleotide, a kit of the present disclosure can include the first oligonucleotide and the second oligonucleotide in separate packaging, e.g. separate tubes.

Methods

Polymerase inhibitors of the present disclosure can be useful for reversible activation of thermostable polymerases for PCR. Exemplary polymerase inhibitors are shown in FIGS. 2-4. In this case, lower melting temperature polymerase inhibitors, where the inhibitor duplex melting temperature is lower than the PCR primer annealing temperature, allow PCR reaction assembly at room temperature while activating polymerase at temperatures at and above the PCR primer annealing temperature, where the reversible nature of the inhibitor additionally improves priming specificity during each PCR cycle. In some embodiments, the melting temperature of the polymerase inhibitor can be from about 5° C. to about 50° C. lower than the PCR primer annealing temperature or an extension temperature. By way of example, but not limitation, the melting temperature of the polymerase inhibitor can be about 5° C., 6° C., 7° C., 8° C., 9° C., 10° C., 11° C., 12° C., 13° C., 14° C., 15° C., 20° C., 25° C., 30° C., 35° C., 40° C., 45° C. or 50° C. below the primer annealing temperature or extension temperature and any range of value therebetween. When used for room temperature setup of PCR reactions, the polymerase inhibitor can be mixed with the polymerase prior to adding primers, substrate polynucleotide and nucleotides to ensure polymerase inhibition.

Polymerase inhibitors of the present disclosure can also be used for temperature controlled inactivation of polymerase activity after PCR or other polymerase based enzymatic incubation and can eliminate a purification step when needed for compatibility with subsequent enzymatic incubations. In such methods, high melting temperature polymerase inhibitors can be used that are above the desired reaction conditions. Exemplary polymerase inhibitors are shown in FIG. 3. By way of example but not limitation, if the ligase used is T4 DNA ligase with reaction conditions between 16-37° C., then a high melting temperature polymerase can be used, where the inhibitor duplex melting temperature is above the reaction conditions. By way of further example but not limitation, if a thermostable DNA ligase is used such as Taq DNA ligase with reaction conditions between 37-75° C., a corresponding higher melting temperature polymerase inhibitor can be used. In some embodiments, the melting temperature of the polymerase inhibitor can be from about 5° C. to about 50° C. higher than the reactions conditions, e.g. enzymatic processing temperature. By way of example, but not limitation, the melting temperature of the polymerase inhibitor can be about 5° C., 6° C., 7° C., 8° C., 9° C., 10° C., 11° C., 12° C., 13° C., 14° C., 15° C., 20° C., 25° C., 30° C., 35° C., 40° C., 45° C. or 50° C. above the reaction conditions, e.g. enzymatic processing temperature, and any range of value therebetween. When used for subsequent enzymatic processing, the inhibitor can be added into the reaction prior to the addition of other reagents such as, by way of example but not limitation, oligonucleotide adaptors that could be processed by the active polymerase.

In some embodiments, a method for performing PCR includes the steps of (i) combining a polymerase inhibitor of the present disclosure with a thermostable polymerase, deoxynucleotides, a substrate polynucleotide, and at least one primer pair comprising a forward primer and a reverse primer sufficient to amplify a target portion of the substrate polynucleotide to form a first reaction mixture under conditions sufficient for the first complementary region and secondary complementary region to form a double-stranded region, and (ii) incubating the first reaction mixture under conditions sufficient to (a) dissociate the first oligonucleotide and the second oligonucleotide of the polymerase inhibitor, (b) allow the forward and reverse primers to anneal to the substrate polynucleotide, and (c) allow the thermostable polymerase to extend the forward and reverse primers to yield PCR amplicons. In some embodiments, the method for performing PCR further includes step (iii) enzymatically processing the PCR amplicons in the first reaction mixture at an enzymatic processing temperature. In some embodiments, the thermostable polymer and polymerase inhibitor are mixed before being combined with the substrate polynucleotide, at least one primer pair and deoxynucleotides. In some embodiments, the temperature for the conditions sufficient in step (ii) is a temperature above the melting temperature of the polymerase inhibitor. In some embodiments, the temperature for the conditions sufficient in step (ii) is at least 5° C. above the melting temperature of the polymerase inhibitor. By way of example but not limitation, the temperature can be about 5° C., 6° C., 7° C., 8° C., 9° C., 10° C., 11° C., 12° C., 13° C., 14° C., 15° C., 20° C., 25° C., 30° C., 35° C., 40° C., 45° C. or 50° C. higher than the melting temperature of the polymerase inhibitor or any range or value therebetween. In some embodiments, the enzymatic processing temperature in step (iii) is below the melting temperature of the polymerase inhibitor. In some embodiments, the enzymatic processing temperature of step (iii) can be at least 5° C. below the melting temperature of the polymerase inhibitor. By way of example but not limitation, the enzymatic processing temperature can be about 5° C., 6° C., 7° C., 8° C., 9° C., 10° C., 11° C., 12° C., 13° C., 14° C., 15° C., 20° C., 25° C., 30° C., 35° C., 40° C., 45° C. or 50° C. lower than the melting temperature of the polymerase inhibitor or any range or value therebetween. In some embodiments, the enzymatic processing step (iii) can include (a) adding a ligase and an adaptor polynucleotide to the first reaction mixture after step (ii) and (b) incubating the PCR amplicons, ligase and adaptor polynucleotide under conditions sufficient to ligate the adaptor polynucleotide to at least a portion of the PCR amplicons. In some embodiments, the method for performing PCR in step (i) can include (a) adding a universal primer to the first reaction mixture, where the forward and reverse primer of each of the at least one primer pair each comprise a 5′ terminal sequence that is not complementary to the substrate polynucleotide, where at least a portion of the universal primer and the 5′ terminal sequence are complementary to a common sequence. In embodiments with the universal primer, the conditions sufficient in step (ii) can be further sufficient to (d) allow the universal primer to anneal to the PCR amplicons; and (e) allow the thermostable polymerase to extend the universal primer to yield universal PCR amplicons. In some embodiments, where the PCR reaction yield universal PCR amplicons, the method can further comprise an enzymatic processing step at an enzymatic processing step as described in the present disclosure.

In some embodiments, a method for inhibiting polymerase in a PCR reaction product can include the steps of (i) adding a polymerase inhibitor of the present disclosure to a reaction product that includes a thermostable polymerase and PCR amplicons and (ii) enzymatically processing the PCR amplicons at an enzymatic processing temperature. In some embodiments, the enzymatic processing temperature of step (iii) can be at least 5° C. below the melting temperature of the polymerase inhibitor. By way of example but not limitation, the enzymatic processing temperature can be about 5° C., 6° C., 7° C., 8° C., 9° C., 10° C., 11° C., 12° C., 13° C., 14° C., 15° C., 20° C., 25° C., 30° C., 35° C., 40° C., 45° C. or 50° C. lower than the melting temperature of the polymerase inhibitor or any range or value therebetween. In some embodiments, the enzymatic processing step (iii) can include (a) adding a ligase and an adaptor polynucleotide to the first reaction mixture after step (ii) and (b) incubating the PCR amplicons, ligase and adaptor polynucleotide under conditions sufficient to ligate the adaptor polynucleotide to at least a portion of the PCR amplicons. I

Generally, the molarity of the polymerase inhibitor should be in excess of the polymerase molecules in order to drive complete polymerase binding and inhibition at temperatures below the melting temperature of the inhibitor duplex. In some embodiments, the polymerase inhibitor is added to a reaction at a molar amount between about 2 and about 1000 times the molar amount of the polymerase, i.e. a molar ratio of polymerase inhibitor to polymerase of between about 1:1 and about 1000:1. By way of example, but not limitation, the molar ratio between the polymerase inhibitor and polymerase can be about 1:1, 2:1, 3:1, 4:1, 5:1, 6:1, 7:1, 8:1, 9:1, 10:1, 20:1, 30:1, 40:1, 50:1, 100:1, 200:1, 300:1, 400:1, 500:1, 600:1, 700:1, 800:1, 900:1, 1000:1 or more and any range or value therebetween. Generally, the molarity of the polymerase inhibitor should be in excess of the substrate polynucleotide when PCR is performed. In some embodiments, where PCR is performed, the molar ratio of polymerase inhibitor to the substrate polynucleotide can, by way of example, but not limitation, between about 2:1 to about 1000:1. By way of example, but not limitation, the molar ratio between the polymerase inhibitor and substrate polynucleotide can be about 1.1:1, 1.5:1, 2:1, 3:1, 4:1, 5:1, 6:1, 7:1, 8:1, 9:1, 10:1, 20:1, 30:1, 40:1, 50:1, 100:1, 200:1, 300:1, 400:1, 500:1, 600:1, 700:1, 800:1, 900:1, 1000:1 or more and any range or value therebetween.

The polymerase inhibitors of the present disclosure can be used in any method where polymerases are used or present. By way of example but not limitation, such methods include PCR and amplicon processing. Non-limiting, exemplary methods that could include a polymerase inhibitor of the present disclosure include the multiplex PCR methods used for the following commercial kits: Swift Biosciences Accel-Amplicon, Swift Biosciences Swift Amplicon HS, Pillar Biosciences ONCO/Reveal BRCA1 & BRCA2 panel, Ion AmpliSeq Cancer Hotspot Panel v2, Illumina TruSeq Amplicon—Cancer Panel, Illumina TruSight Panels, AmpliSeq for Illumina Comprehensive Cancer Panel, Qiagen QuantiFast Kits. Non-limiting, exemplary methods that could include a polymerase inhibitor of the present disclosure include the methods used for the following commercial kits: Swift Biosciences Swift 2S Turbo Library kit, Swift 2S library kit, Roche Kapa HiFi, Roche Kapa Hyper, New England Biolabs NEBNext® Multiplex Oligos.

### EXAMPLES

The following examples, as described below, use materials with the sequences in Tables 1-3 as provided below.

TABLE 1
Oligonucleotides used in Example 1 and 2

| | SEQ | |
|---|---|---|
| Sequence | ID | |
| Name | NO | Sequence (5′-3′) |
| 18-57 | 1 | AGTCrUrUrUrUGGTTTTTTTTTTTTGG |
| 18-58 | 2 | CCAAAAAAAAAAAACCrUrUrUrUCTGA |

TABLE 2
Target-Specific Oligonucleotides
used in Example 2

| | | Concen- |
|---|---|---|
| SEQ | | tration |
| ID | | in Reagent |
| NO | Sequence (5′-3′) | G1 (nM) |
| 3 | CATCGTGGGGCCTGGTGG | 34 |
| 4 | TGGCTACAAGGAGCGGCC | 34 |
| 5 | CAGAGAAGTTGTTGAGGGGAGC | 51 |
| 6 | CATCAAGACCTGGCGGCC | 51 |
| 7 | CATCATTCTTGAGGAGGAAGTAGCG | 34 |
| 8 | AGGCACATCTGTCCTGGCA | 34 |
| 9 | TCTAGTTTTTAGAAGTACCCAGATTTGACA | 130 |
| 10 | TTGTGCCTCCACTGTCCAAAAA | 130 |
| 11 | ACTCATTTTGTGTAGGAAAGGTACAATGAT | 86 |
| 12 | TCCACAGATTTATATCATCCAGGCCT | 86 |
| 13 | GTAGTTCTGTTAAAGTTCATGGCTTTTGT | 34 |
| 14 | TTTCAGTGCACGCAGGGC | 34 |
| 15 | CGAGAGCTGGAGTTGGATGAATT | 51 |
| 16 | GCCAGAGGGAACAAAGTCGG | 51 |
| 17 | GTGGAGAAGAACATGATATGTGGGT | 51 |
| 18 | TGTCTAAGTTTTTCAAGCACAGGGT | 51 |
| 19 | TGGACCACACAGGAGAATATGGA | 51 |
| 20 | GCTCACCTTAACAAGCTGTCTCC | 51 |
| 21 | TTCTTTAAGTGCAAATAGTGTATCTGACCT | 51 |
| 22 | ATTTACACCTCCTGCTAAGCGAAAT | 51 |
| 23 | GATTGTGTAGGTTCCGATGGCA | 51 |
| 24 | AAAGACAAAATCCCAAATAAAGCAGAAAGA | 51 |
| 25 | TGGAATCAGTGATTTCAGATTGTTTGTTT | 130 |
| 26 | AGATCTATATGTACAAGTTCTGCTGACTG | 130 |
| 27 | AAAGTAGCTGAACGTGTCTTAATGAGA | 51 |
| 28 | TCCTGGGAAAAGTCGGCTGA | 51 |
| 29 | GCAGGCCATAGACCCCAAAAA | 51 |
| 30 | CCCTACTTAAAGTATGTTGGCAGGTT | 51 |
| 31 | TCTTTACATGGCTTTTGGTCTTCTAAGT | 103 |
| 32 | CCATTCTGGCACGCTTTGGAA | 103 |
| 33 | CTCACTATGAAAAACTGTAAAGCTGCAA | 103 |
| 34 | TTATTAAGATGCAGCTACTACCCAGC | 103 |
| 35 | TCCATGAATCTATTTAACGATTACCCTGAT | 51 |
| 36 | GAGGCCTCTTATACTGCCAAATCAA | 51 |
| 37 | GCCATTTGACCGTGGAGAAGT | 51 |
| 38 | ACTTTGGCTCTCTCCAGGTTCG | 51 |
| 39 | TTGACTACAGCATGCTCCTGC | 51 |
| 40 | TCCTCCACCTCCATTAGATTTCCA | 51 |
| 41 | TTTTTCAGTGGAGGTTAACATTCATCAAG | 51 |
| 42 | GAAACTATGTCCAGTCTTTGTGGCT | 51 |
| 43 | AGTTCGATCAGCAGCTGTTACC | 51 |
| 44 | AAGGCTGTAGATAGGCCAGCA | 51 |
| 45 | GGGAGATTTATAAGATGACAACAGATCCA | 51 |
| 46 | AGTCATGACCCACAGCAAACAG | 51 |
| 47 | TTGGAGCCGCAGCCTCTC | 86 |
| 48 | TGGAAGAAGGGAAGCGGTGA | 86 |
| 49 | ACTCCAGCCCGCTCCAGC | 51 |
| 50 | CCCTCGCAAGTCAGGGGA | 51 |
| 51 | CCTGGGCAGAGGTGAGGG | 51 |
| 52 | CTGCGTAAATTCCAAGGGGTGT | 51 |
| 53 | CAGGCTTTGTGGATTTGACCCT | 51 |
| 54 | CTGTCCAAGAGCAAGTTAGGAGC | 51 |
| 55 | GGAGCACCCAGGGAAGCT | 51 |
| 56 | TCTGAAGCTTCACCGAGAGATGA | 51 |
| 57 | CCAGGGAAAATGTGTAGAGGGC | 51 |
| 58 | TGTGTTATCAACTCACCAGAATTAAGCA | 51 |
| 59 | CCAGTGGATTTTTATGAATGTGAACCC | 130 |
| 60 | CAGCATGTCGAAGATCTCCACC | 130 |
| 61 | CAGGGAGAGGAGTTTGTGTGC | 51 |
| 62 | GACATTATGCCTTTGGAGTGGGT | 51 |
| 63 | TCCTAGACCTCATCCTCTTTGAGC | 51 |
| 64 | TGTCTGTGATCTTGTCCAGGACT | 51 |
| 65 | GGCCTGACCCTGCAGCAG | 51 |
| 66 | CCCGGCCAGCTGTCACAT | 51 |
| 67 | TCAGCACTCCTGGGGCTC | 51 |
| 68 | TCCAGCATCTCCAGCAGCA | 51 |
| 69 | TGCAAGAACGTGGTGCCC | 27 |
| 70 | CCAAGTGGCTTTGGTCCGTC | 27 |
| 71 | ATGCGCCCACTAGCCGTG | 27 |
| 72 | GGAAACCCTCTGCCTCCCC | 27 |
| 73 | GGGCTCTACTTCATCGCATTCC | 51 |
| 74 | GCTAAAGTGGTGCATGATGAGGG | 51 |
| 75 | GAACCCTATTGGTGTTACT | 172 |
| 76 | AGTCAAACTCCAACTCTAAG | 172 |
| 77 | TCCCCGAAATTCTACCCAAATTGC | 86 |
| 78 | GTGGACTTTCTGAGAGAAAACAATTTAAGT | 86 |
| 79 | CAACCTTTTGAACAGCATGCAAGA | 51 |
| 80 | ACCTGGGCTACTTCATCTCTAGAAT | 51 |
| 81 | TCTTTTCTCAAGTTGGCCTGAATCA | 86 |
| 82 | TGACGATCTCCAATTCCCAAAATGA | 86 |
| 83 | CGTTCATGTGCTGGATACTGTGT | 51 |
| 84 | AACACCAAAACATTTTAAACAGAGAAAACC | 51 |
| 85 | CATGGTGAAAGACGATGGACAAGT | 257 |
| 86 | AGTGTCCAAAATCTATATGAAACAGCTTTC | 257 |
| 87 | TGATGACATTGCATACATTCGAAAGAC | 51 |
| 88 | GCATGCTGTTTAATTGTGTGGAAGA | 51 |
| 89 | CGACAGCATGCCAATCTCTTCA | 86 |
| 90 | CATGAAATACTCCAAAGCCTCTTGC | 86 |
| 91 | CCTGAAGGTATTAACATCATTTGCTCCA | 130 |
| 92 | CCAGAGCCAAGCATCATTGAGA | 130 |
| 93 | TCATGGTGGCTGGACAACAAAA | 51 |
| 94 | CGGTCTTTGCCTGCTGAGAG | 51 |
| 95 | CTCTGCCAGGAGCCGGAG | 52 |
| 96 | CCTACTCCGCCCAGGGAC | 52 |
| 97 | GGCGCTGTTGGTTTCGGT | 52 |
| 98 | GTGAACGTGTAGCTCTCGGC | 52 |
| 99 | ACAGTCAAAAGGCCTCTACGGT | 52 |
| 100 | ATACCTGATGGGGCGGGG | 52 |
| 101 | CGCTCTTTGGAGAAGGAATGCT | 52 |
| 102 | GTGCCCACTTTGAATCGGGT | 52 |
| 103 | TTCCACCAAAGTCACGCTGAAT | 52 |
| 104 | GTCAACGGTACCAAGGCTGAG | 52 |
| 105 | TGGATAGAGAACGCATTGCCAC | 52 |
| 106 | TTAAGCTCCTCATGTGTTCAGAGC | 52 |
| 107 | GCATCACTGGCCAAGGAGC | 52 |
| 108 | CAACAAGCTTCTAAGAGATACTTACAGTGT | 52 |
| 109 | CCAGTGTTGGGATCCTTCTTTACTAAT | 87 |
| 110 | CTTTCAATAATAAAGACACCAACAGGGG | 87 |
| 111 | TTCTCTGGGAGGGATTTGGCA | 87 |
| 112 | TGTCTTTGTTGGATTTGATCTGAAGACA | 87 |
| 113 | AGAGAGACTGGGTTATTCCTCCC | 52 |
| 114 | TTCCCTTTCTCTCCTTGGTACTTCT | 52 |
| 115 | CATCTTCTTTCCTTTTAGGCCTCCG | 52 |
| 116 | GGGCCTTTTTCATTTTCTGGGC | 52 |
| 117 | TGTCTGGCTAGGTTGGACTGT | 52 |
| 118 | GCCAGGAGAGGAGTTGGGAA | 52 |
| 119 | CGCTGTGTCATCCAACGGG | 52 |
| 120 | TGGATATACCTGGAAGAGCACCTT | 52 |
| 121 | CCCTTCTCCCATGTTTTCTTCCTC | 87 |
| 122 | CGGTTACCGTGATCAAAATCTCCA | 87 |
| 123 | AGCCCGAATTCACCCAGGA | 52 |
| 124 | AACCTTTGGGCTTGGACAACA | 52 |
| 125 | CCCAGTCCCAAAGTGCAGC | 52 |
| 126 | GGCTGAGGATGGTGTAAGCG | 52 |
| 127 | CCACAGACGCGGACGATG | 52 |
| 128 | TCCAGCCCAGTGGTGACC | 52 |
| 129 | ACAGGAACACAGGAGTCATCAGT | 52 |
| 130 | TGACAACTGGCCTAGCAGGA | 52 |
| 131 | CAAAGGTGGCTAGTGTTCCTGG | 52 |
| 132 | TGGTGTCAGTGACTGTGATCACA | 52 |
| 133 | GAGGGGTTAAGCACAACAGCA | 52 |
| 134 | TGGTGACACTTAGTTCATGAACAACA | 52 |
| 135 | GCCCCCTGAGACTCAGCT | 52 |
| 136 | TGGGGGCATCAGCATCAGT | 52 |
| 137 | GCTAACGTCGTAATCACCACACT | 52 |
| 138 | AATGCCATCGTTGTTCACTGGA | 52 |
| 139 | AC CATATTGAATGATGATGGTGGACA | 52 |
| 140 | CCAGGGGACAAGGGTATGAACA | 52 |
| 141 | CTGCCCAGGAGCCAGACA | 52 |
| 142 | ACAGACAAATGACAAAATGCCATGAA | 52 |
| 143 | GCCCCCATCTTTGTGCCTC | 174 |
| 144 | GCAAGTCAGTTGAAAAATCCTCACAC | 174 |
| 145 | GCAGTGACGAATGTGGTACC TT | 52 |
| 146 | GCCCACGCCAAAGTCCTC | 52 |
| 147 | TTCATTGTTTCTGCTCTCTAGGGC | 52 |
| 148 | CACATCCAGCACATCCACGG | 52 |
| 149 | GCTACATGTTGTTTGCTGGTCCT | 52 |
| 150 | TCCCTGTCCAGCTCAGCC | 52 |
| 151 | TCCGGACACTGGTGCCAT | 52 |
| 152 | GGTCGAGGCAGCAAAGGC | 52 |
| 153 | TCTAGACTTGGTCTGGTGGAAGG | 52 |
| 154 | TGTCATTCACATCAGACAGGATCAG | 52 |
| 155 | CCCCCATACCAGAACCTCGA | 52 |
| 156 | GGTCCAGTTGGCACTCGC | 52 |
| 157 | CCCAATACATCTCCCTTCACAGC | 52 |
| 158 | AGGTGGGGATCTGGGGGA | 52 |
| 159 | TGAGCTTTTTATTTTCCTCCCCTGG | 87 |
| 160 | TCTGGTTATCCATGAGCTTGAGATTG | 87 |
| 161 | TGGCCTTAGAGGTGGGTGAC | 52 |
| 162 | TCCTGCTTCGACAGGCTGT | 52 |
| 163 | GC GTGTGTGACTGTGAAGGG | 52 |
| 164 | CAGCTGGACTTACTTAGCAAAGCA | 52 |
| 165 | GAGGATGACACCCGGGACA | 52 |
| 166 | GCTGTTTCAAATGCCTACCTCTTACT | 52 |
| 167 | CCCCACCATCCCAGTTCTGA | 52 |
| 168 | CCTCTTCTCCGCCTCCTTCT | 52 |
| 169 | CTAACTGCCCCCTGTCTGGT | 52 |
| 170 | GCTCTCCTCCGAAGAAACAGC | 52 |
| 171 | AACTGAACATAGCCCTGTGTGTATG | 52 |
| 172 | GGGTTGGTGCAACGTCGT | 52 |
| 173 | TATCTTCCCCGCCCTGCC | 52 |
| 174 | AGACTCTTTTTCTCATTTTTGACACAACTC | 52 |
| 175 | GCTGCTAGTCTGAGCTCCCT | 27 |
| 176 | GCCTCTCTCGAGTCCCCTAGT | 27 |
| 177 | TGTCGTACCTTACATATTGCTAGACTTC | 52 |
| 178 | AGAGAATCATAAGGCGGGGCT | 52 |
| 179 | CTTCAAGAAGCTGGCTGACATGT | 52 |
| 180 | ACTCATCTCAAGGGAAGGGAGC | 52 |
| 181 | GCACCTCCCGCTCCTGGA | 52 |
| 182 | GTTGGAGGCAGTAGAAGGGGA | 52 |
| 183 | CCTGTCACCATTTCCAGGGC | 87 |
| 184 | GGCGCACGGGAGGTTTAAA | 87 |
| 185 | TGGCACATCCAGGGACCC | 52 |
| 186 | GGCAGCGGAATGGGGAGA | 52 |
| 187 | CCAGAGCCATTTCCATCCTGC | 52 |
| 188 | TCCTCTTGATATCTCCTTTTGTTTCTGC | 52 |
| 189 | ATAGTATTAATGTAATTTCAAATGTTAGCT | 262 |
| 190 | GCAAGCATACAAATAAGAAAACATACTTA | 262 |
| 191 | TGTGCATATTTATTACATCGGGGCA | 174 |
| 192 | CCAATAAATTCTCAGATCCAGGAAGAGG | 174 |
| 193 | CTGTCCACCAGGGAGTAAC | 52 |
| 194 | TTCCGCCACTGAACATTGG | 52 |
| 195 | CTTCCACAAACAGAACAAGATGCTAAA | 132 |
| 196 | AAAACACCTGCAGATCTAATAGAAAACAAA | 132 |
| 197 | CGGGAAGACAAGTTCATGTACTTTGA | 87 |
| 198 | TGTCCTTATTTTGGATATTTCTCCCAATGA | 87 |
| 199 | ACAGAATCCATATTTCGTGTATATTGCTGA | 52 |
| 200 | CACCTTTAGCTGGCAGACCAC | 52 |
| 201 | TAGATATTCTGACACCACTGACTCTGATCC | 152 |
| 202 | AAGGTCCATTTTCAGTTTATTCAAGTTTATTT | 152 |
| 203 | AGATGAGTCATATTTGTGGGTTTTCATTTT | 52 |
| 204 | TCAGGTTCATTGTCACTAACATCTGG | 52 |
| 205 | GATAGCATTTGCAGTATAGAGCGTG | 52 |
| 206 | AACCCCCACAAAATGTTTAATTTAAC | 52 |
| 207 | TGGAGAAGGGAAGTCGGAACA | 52 |
| 208 | CACCGACATCAGCTCGCC | 52 |
| 209 | GCAGCAGCTGGGCATGTT | 52 |
| 210 | GCAGGTCCCCCATCAGGT | 52 |
| 211 | CATCTACCAGCCGCGCCG | 52 |
| 212 | TGAGGATCTTGACGGCCCTC | 52 |
| 213 | AGGAGGTGCTGGACTCGG | 52 |
| 214 | ACCCCAGCAAGCCATACTTACT | 52 |
| 215 | CGGGGAGGCCAACGTGAA | 52 |
| 216 | GGTCCAGCTCAGGGTGTTAAGA | 52 |
| 217 | GGGCCTGTGGTGTTTGGG | 52 |
| 218 | TGCCCTGGCTATGCAGGT | 52 |
| 219 | CGCAGGTACTTCTGTCAGCTG | 52 |
| 220 | GCCTACCTCGGCCACGCC | 35 |
| 221 | ACCGGTGGCACCCTCAAA | 35 |
| 222 | GAACGGGTGCAGTGCCTG | 52 |
| 223 | CTCTGTCCCTGGGGTAGAGC | 52 |
| 224 | ACAACTTGTAGATGTTGTCCCCTTC | 52 |
| 225 | AGCTACAACATCACCACGGGT | 52 |
| 226 | AGGCTCCCACCTTTCAGCA | 52 |
| 227 | CATCCCGGGCGACTGTGG | 52 |
| 228 | GGGGTCTCGGGGCCAATA | 52 |
| 229 | TGGTGCCAGCCTGACAGG | 52 |
| 230 | CAGTCATGCTGCGCCACC | 52 |
| 231 | CGAGCCCAGACACCAAGGA | 27 |
| 232 | GCCAGACTCACCGGGCAC | 27 |
| 233 | TGACATCATCTACACTCAGGACTTCA | 52 |
| 234 | AC CAGAGGGCAGAAGCTGT | 52 |
| 235 | CTCTGTCTCCTTCCTCTTCCTAC | 52 |
| 236 | GTGCTGTGACTGCTTGTAGA | 52 |
| 237 | CTGTGCAGCTGTGGGTTGA | 52 |
| 238 | CATGACGGAGGTTGTGAGG | 52 |
| 239 | CAGGTAGGACCTGATTTCCTTAC | 52 |
| 240 | TTCTTGCGGAGATTCTCTTCC | 52 |
| 241 | TGGGACGGAACAGCTTTGAG | 52 |
| 242 | CCACCGCTTCTTGTCCTG | 52 |
| 243 | GGGTGCAGTTATGCCTCAG | 52 |
| 244 | AGACTTAGTACCTGAAGGGTGA | 52 |
| 245 | TAGCACTGCCCAACAACACC | 52 |
| 246 | CGGCATTTTGAGTGTTAGACTGG | 52 |
| 247 | TTACTTCTCCCCCTCCTCTG | 52 |
| 248 | CTTCCCAGCCTGGGCATC | 52 |
| 249 | GCTGAATGAGGCCTTGGAAC | 52 |
| 250 | CTTTCCAACCTAGGAAGGCAG | 52 |
| 251 | TCCTCCCTGCTTCTGTCTC | 52 |
| 252 | CTGTCAGTGGGGAACAAGAAG | 52 |
| 253 | TCTTGCAGCAGCCAGACT | 52 |
| 254 | CCTGCCCTTCCAATGGATC | 52 |
| 255 | CCCCTAGCAGAGACCTGT | 523 |
| 256 | GCCCAACCCTTGTCCTTAC | 523 |
| 257 | CTGACTGCTCTTTTCACCCAT | 52 |
| 258 | GAGCAGCCTCTGGCATTCTG | 52 |
| 259 | TGAAGACCCAGGTCCAGATGA | 52 |
| 260 | GCTGCCCTGGTAGGTTTTCTG | 52 |
| 261 | CTGGCCCCTGTCATCTTCTG | 52 |
| 262 | CAGGCATTGAAGTCTCATGGA | 52 |
| 263 | GCTCACCATCGCTATCTGAG | 52 |
| 264 | AGCAATCAGTGAGGAATCAGAG | 52 |
| 265 | AGCTGGGGCTGGAGAGA | 52 |
| 266 | GTCATCCAAATACTCCACACGCA | 52 |
| 267 | GCATCTTATCCGAGTGGAAGG | 52 |
| 268 | CACTGACAACCACCCTTAACC | 52 |
| 269 | CGCACTGGCCTCATCTTG | 52 |
| 270 | CTTCCAGTGTGATGATGGTGAG | 52 |
| 271 | CATGTGTAACAGTTCCTGCATG | 52 |
| 272 | GGTCAGAGGCAAGCAGAG | 52 |
| 273 | CCTGGTTGTAGCTAACTAACTTC | 87 |
| 274 | ACCATCGTAAGTCAAGTAGCATC | 87 |
| 275 | ATGGTTCTATGACTTTGCCTGA | 52 |
| 276 | AGCAGGCTAGGCTAAGCTATG | 52 |
| 277 | TGATTTAGGTTTCTGCTTTGGGACA | 436 |
| 278 | TGCCCCACAGTTCACCTGA | 436 |
| 279 | CAGAACAATGCCTCCACGACC | 52 |
| 280 | ATGGTTATTAATGTAGCCTCACGGAG | 52 |
| 281 | TGTTTACTACCAAATGGAATGATAGTGACT | 174 |
| 282 | TGAAGAAGTTGATGGAGGGGGT | 174 |
| 283 | AGTGTTACTCAAGAAGCAGAAAGGG | 52 |
| 284 | TCATACCAATTTCTCGATTGAGGATCTT | 52 |
| 285 | TGCACCATTGATGTCTACATGATCA | 44 |
| 286 | GGTCCCCTTTCATGCCCCT | 44 |
| 287 | CAGCAAGCACACAGGGCC | 44 |
| 288 | CACAAAGCGCTGGGGGTC | 44 |
| 289 | GAATTCTCCCGCATGGCCAG | 44 |
| 290 | AGGGGCCTGGCATACTGG | 44 |
| 291 | TGCCTCTCCTTCCTCCACAG | 44 |
| 292 | TGGACAGAAGAAGCCCTGCT | 44 |
| 293 | GGACCTGGTGGATGCTGAGG | 44 |
| 294 | ACACAGTGTGACCGAGGGC | 44 |
| 295 | TGGTCCACCACAGGCACC | 73 |
| 296 | TGGGGTTTCCTTGAGAGGTGA | 73 |
| 297 | TCACCTTCCATGGAGTCCCC | 44 |
| 298 | CCTGGGGGCCTCCTCTTC | 44 |
| 299 | GGACCTGACACTAGGGCTGG | 44 |
| 300 | GTGTGGGGAGGCTTTGCAG | 44 |
| 301 | CCAGCCCTCTACAGCGGT | 44 |
| 302 | CACCTCCCCTGCCCATCA | 44 |
| 303 | CAGCCTGGTATGGAGTCCAGT | 44 |
| 304 | TTCTGAAAGGTCAAGAGAAGGTGAC | 44 |
| 305 | AGTGGCAGAGACACCGGG | 44 |
| 306 | TCCAGAGTGGCACCAGCA | 44 |
| 307 | ACGTTTTTGCCTTTGGGGGT | 44 |
| 308 | GCTCTGGTGGGTCCTGGT | 44 |
| 309 | TCCTCCTGCCTTCAGCCC | 44 |
| 310 | TGGCACGTCCAGACCCAG | 44 |
| 311 | CCTACGGCAGAGAACCCAGA | 44 |
| 312 | GAAGTGGTCGGAGGGCCC | 44 |
| 313 | GAGCAGGGAAGGCCTGACT | 44 |
| 314 | ACGAGGCTGGACCCCTTC | 44 |
| 315 | TCCTTCCTGCTTGAGTTCCCA | 44 |
| 316 | GGAAAGGGCCAAGCTGGG | 44 |
| 317 | AAGCTGGCCTGAGAGGGG | 44 |
| 318 | AAGCACTCTGTACAAAGCCTGG | 44 |
| 319 | GGAAGGAACAGCAATGGTGTCA | 44 |
| 320 | CCCACACTTGCCTCCCCA | 44 |
| 321 | CCAGGGGGAGAATGGGTGT | 44 |
| 322 | ACAGAGCCACCCCCAGAC | 44 |
| 323 | AATTTGTAGACCCTCTTAAGATCATGCT | 183 |
| 324 | TGGTTTTCCCACCACATCCTCT | 183 |
| 325 | CCGCAGTGAGCACCATGG | 44 |
| 326 | ATCCACAGGGCAGGGTCC | 44 |
| 327 | GATGGGGTGGCCAGGTCT | 23 |
| 328 | CCCTGGTAGAGGTGGCGG | 23 |
| 329 | CCCGAGACCCACCTGGAC | 23 |
| 330 | CAGGGCCTGGCTGGGTTG | 23 |
| 331 | CAACCAAGTGAGGCAGGTCC | 29 |
| 332 | GTGGTATTGTTCAGCGGGTCTC | 29 |
| 333 | TATGCCCTGGCCGTGCTA | 44 |
| 334 | ACCAAGAGAAGGTTTCAATGACGG | 44 |
| 335 | TGGGGAATCTGGGGGTTGTT | 44 |
| 336 | CCGCTGGATCAAGACCCCT | 44 |
| 337 | AAGGGTCCTCTGATCATTGCTCA | 44 |
| 338 | AGTGTGAGAGCCAGCTGGT | 44 |
| 339 | AGGACACGATTTTGTGGAAGGAC | 44 |
| 340 | GTTTGCGGCTGGGGTCAG | 44 |
| 341 | AACCCGTCCTCTCGCTGTT | 44 |
| 342 | CCTCAGAACTCTCTCCCCAGC | 44 |
| 343 | TCCGATGTGTAAGGGCTCCC | 44 |
| 344 | CCCCTCCCATGTCACCTGT | 44 |
| 345 | CCTGGGCCAGGTAGTCTCC | 44 |
| 346 | ACAGCCACCGGCACAGAC | 44 |
| 347 | GGGCCCCAAGCACTCTGA | 73 |
| 348 | TGGCTGGCTTTCACTGTGC | 73 |
| 349 | ACTGCCCTATTGCCCCTGG | 44 |
| 350 | CGTGTCTGTGTTGTAGGTGACC | 44 |
| 351 | CAGTGGCATCTGTGAGCTGC | 44 |
| 352 | CTGTGTTTCTCCCTGGCACTC | 44 |
| 353 | GCCCCCTGCACAACCAAG | 44 |
| 354 | GCTGGGCCAGGCTGCATG | 44 |
| 355 | GCACTTGCGAGAGGTGAGG | 44 |
| 356 | TGTCTGCCCTGACACTGTCT | 44 |
| 357 | TGTCCACCCTGTTCCTGGC | 44 |
| 358 | TGCAGAGACAGAGCCCACC | 44 |
| 359 | CCAGAGCAGCTCCAAGTGTTT | 44 |
| 360 | TGCTGAGATGTATAGGTAACCTGCA | 44 |
| 361 | GGAGTCCTTGTCCTGTCCCC | 23 |
| 362 | TTGTGCAGAATTCGTCCCCG | 23 |
| 363 | TGCCTGACCTCAGCGTCTT | 44 |
| 364 | GGAGTACTCCCTCAGGCCC | 44 |
| 365 | CCTTTCTCCCATAGTGGCGC | 44 |
| 366 | GTGTTATGGTGGATGAGGGCC | 44 |
| 367 | GAGGGAACTGGGCAGTGGA | 23 |
| 368 | ACCACACTCGTCCTCTGGC | 23 |
| 369 | CACCAAGCTCTGCTCCACAC | 44 |
| 370 | TCTGCACAAGTCCAAGAACGC | 44 |
| 371 | CCACAGCCATGCCCACAG | 44 |
| 372 | CACAGCTGGTGGCAGGCC | 44 |
| 373 | CCCGAGGGCACTGCTGGG | 44 |
| 374 | CATGCACCCCTCCAGCCA | 44 |
| 375 | GCCGAGTACTGCAGGGGTA | 44 |
| 376 | GGTGGCACGGCAAACAGT | 44 |
| 377 | TCTCAGGCTCCCCAGGGA | 44 |
| 378 | CAATCCCCCTCGCTGCCC | 44 |
| 379 | CCTTGGGAAGCACAAAGGGG | 44 |
| 380 | ACGCAGAAGGGAGGGTCC | 44 |
| 381 | CCAGTGTGTGGCCTGTGC | 44 |
| 382 | GGGTGCAGTTGATGGGGC | 44 |
| 383 | GATGAGGAGGGCGCATGC | 44 |
| 384 | GATATGACAAAGGGAGAGTTGGTCC | 73 |
| 385 | TTCTGCCTTTGTCAAATGGGGAT | 73 |
| 386 | AGCCCTTGTCATCCAGGTCC | 44 |
| 387 | GAGACTGTTTCTCCTGCAGCTG | 44 |
| 388 | ACAGCAGTGACCACCCAGC | 44 |
| 389 | CCCAGCCCTCTGACGTCC | 44 |
| 390 | CACCTCCGTTTCCTGCAGC | 44 |
| 391 | CCGGAAGTACACGATGCGG | 44 |
| 392 | AGGTGTCAGCGGCTCCAC | 44 |
| 393 | ACCACCCCCTCACCCCAG | 44 |
| 394 | GGCCCTGACCTTGTAGACTGT | 44 |
| 395 | GGTGCTTGGATCTGGCGC | 44 |
| 396 | CCCAAACACTGCCTCCAGC | 44 |
| 397 | AGGTAGGATCCAGCCCACG | 44 |
| 398 | TTTGTTGGCTTTGGGGGATGT | 44 |
| 399 | TCCAGTGGCCATCAAAGTGTTG | 44 |
| 400 | TGAAGAGAGACCAGAGCCCAG | 44 |
| 401 | TGGGGGTGTGTGGTCTCC | 44 |
| 402 | AAGCTGTGTCACCAGCTGC | 44 |
| 403 | TGTCTCCCGCCTTCTGGG | 44 |
| 404 | AGCAGGTCCTGGGAGCCC | 44 |
| 405 | GCAGGTCTCTCCGGAGCA | 44 |
| 406 | AGCCGCACATCCTCCAGG | 44 |
| 407 | CCAGAAGGTCTACATGGGTGCT | 44 |
| 408 | AGCCAGCCCGAAGTCTGT | 44 |
| 409 | CGTGCTGGTCAAGAGTCCCA | 44 |
| 410 | CACCACTCCACCCAGCCT | 44 |
| 411 | GGCCACCTCCCCACAACA | 44 |
| 412 | CCCCATCACACACCATAACTCC | 44 |
| 413 | GTCCATTCTCCGCCGGCG | 44 |
| 414 | CACATGCTGAGGTGGCCC | 44 |
| 415 | AAGCTCCCTCTGGCCCTC | 44 |
| 416 | CAGCCGCTCCCCCTTTTC | 44 |
| 417 | GCTGATGACTTTTGGGGCCA | 44 |
| 418 | CACAGCTCAGCCACGCAC | 44 |
| 419 | AGGCTGTTGGAAGCTGCTTG | 61 |
| 420 | GGTCAGCATTATGAAGGTCCACTG | 61 |
| 421 | TTTTTAATGATGCTTTCTGGCTGGATTT | 511 |
| 422 | AATTCCATTACCTTTTCTCTTGATCATCCA | 511 |
| 423 | ACTCTATGCAGAAATCTATGCAGATAAGAA | 306 |
| 424 | ATGGGGAACAGGAGGCAAAATAAA | 306 |
| 425 | TGACCTGAGACAAGATGCTGTCA | 511 |
| 426 | TGTTTTTGGTGAACTAACAGAAGTACAAAT | 511 |
| 427 | GGCTCAGCATACTACACATGAGAG | 511 |
| 428 | GGTTAACAGAGTTTCCTGAGAGTTTCT | 511 |
| 429 | GTGTTTGACTCTAGATGCTGTGAG | 204 |
| 430 | CCTGATGAGATACACAGTCTACC | 204 |
| 431 | CATTTGGATAAAGACACTGACTTGTGC | 73 |
| 432 | AGCACTCTTTAGATAAACAGGTCATAAACA | 73 |
| 433 | CTCTTCCTCGGCTTCTCCTGA | 49 |
| 434 | CCTGGAGCTGCAGCCGCC | 49 |
| 435 | TCTTCCTAAGTGCAAAAGATAACTTTATAT | 972 |
| 436 | TAGTACAGTACATTCATACCTACCTCTGC | 972 |
| 437 | AGACCAGTGGCACTGTTGTTTC | 63 |
| 438 | ATGGTTAAGAAAACTGTTCCAATACATGG | 63 |
| 439 | TGGTATGTATTTAACCATGCAGATCCTC | 49 |
| 440 | CCACACACAGGTAACGGCTG | 49 |
| 441 | CCCTGATGCTCATGTGGCTG | 1361 |
| 442 | ACTCCTGGATATTGGCACTGGT | 1361 |
| 443 | GC CGGAGAGCTTTGATGGG | 486 |
| 444 | GCTTTCTTTGCATTCTTGATCCCC | 486 |
| 445 | CCGTGGGCCCCCTTTGTC | 1701 |
| 446 | CCCAAGACCACGACCAGCA | 1701 |
| 447 | TGGTGACCTGGGAATGGGG | 49 |
| 448 | CATCAGTCTCAGAGGGCAGGG | 49 |
| 449 | GGCCCTGCCCAATGAGACT | 49 |
| 450 | CGCTTTTGTTCTTAGACACTCCCT | 49 |
| 451 | TGGACTTGGTGATAGACATGTACAGA | 531 |
| 452 | TGGTAGGCAAACAACATTCCATGA | 531 |
| 453 | TTCCCAAGGCCTTTAAACTGTTCA | 40 |
| 454 | ACAGTGCCTTCTTCCACTCCT | 40 |
| 455 | GGACAGCCTATTTTTCCCTCGAC | 40 |
| 456 | CTGTAGGTGGAGTCCCAGGC | 40 |
| 457 | ACACCGGGGTAACATCCACC | 40 |
| 458 | CAACCCCAAACTGTCCCACG | 40 |
| 459 | AGCGGCTGATACTGACCCC | 40 |
| 460 | TCAAGTAGTCATAGTCCTGGTCTTTGT | 40 |
| 461 | TGTCAGTTCAAATCCCTGTTGCA | 40 |
| 462 | AGCCAGGCACATTCTAGAAGGT | 40 |
| 463 | ACCTGTTAAGTTTGTATGCAACATTTCTAA | 133 |
| 464 | AGCTGTGGTGGGTTATGGTCT | 133 |
| 465 | CCCACCAATGCCAGCCTG | 40 |
| 466 | AACAAGAGAGGAAACAGAAGGGC | 40 |
| 467 | CTGCTTCCCCCTCCCAGG | 27 |
| 468 | CAAAGAGCTGGGTGCCTCG | 27 |
| 469 | TTGCCCAACAGTGACGCG | 57 |
| 470 | GCAGAGGCACATACCAGGC | 57 |
| 471 | TGTGACTGCCTGTCCCTGT | 40 |
| 472 | AAGGCAGCTCGGCAGGAA | 40 |
| 473 | CATGGTGGTGCACGAAGGG | 40 |
| 474 | ACCGCTGTGTTCCATCCTCT | 40 |
| 475 | CTGCGGTCCCTTCCTCCT | 40 |
| 476 | GGAACTGGCTGCAGTTGACA | 40 |
| 477 | TGGCTGCCTCTTAGACCATGT | 40 |
| 478 | AGCCCCTTGTGGACATAGGG | 40 |
| 479 | AGAGCACCCTCCTGCAGAG | 40 |
| 480 | TCCAAGGGACTGGCTGGG | 40 |
| 481 | AAAACAGCTAGGCACCGGC | 40 |
| 482 | AACTGGATGTCTGGCTCCTCA | 40 |
| 483 | TGCTATGGGATTTCCTGCAGAAAGAC | 437 |
| 484 | CACAACATGAATATAAACATCAATATTTGAA | 437 |
| 485 | TGGATTCAAAGCATAAAAACCATTACAAGA | 170 |
| 486 | ACTCTACCTCACTCTAACAAGCAGA | 170 |
| 487 | TTTAGTTGTGCTGAAAGACATTATGACAC | 243 |
| 488 | TCTCACTCGATAATCTGGATGACTCA | 243 |
| 489 | TCTCTTAGGTTCTCCAGTTGCTACT | 73 |
| 490 | TGATGTTTATGACCTGAGGCTTTGG | 73 |
| 491 | GGCAGCCGTTCGGAGGAT | 49 |
| 492 | TTGGCTCTGGACCGCAGC | 49 |
| 493 | CAACCATCCAGCAGCCGC | 49 |
| 494 | CAGAAGCTGCTGGTGGCG | 49 |
| 495 | CAAATTCCTGCCATTCTGGGGA | 45 |
| 496 | CATTTCCAGGAAATAAACCTCCTCCA | 45 |
| 497 | CCAGCTGCACAGGGGCCT | 45 |
| 498 | TTCCACGTGGATTACTTACTTCATCAA | 45 |
| 499 | TCCAGCACCCTGAAGTCTCTG | 45 |
| 500 | GAGGAGGAGCTGGGCCAG | 45 |
| 501 | GACAGCCATCATCAAAGAGATCGT | 45 |
| 502 | TCGCATCCGTCTACTCCCAC | 45 |
| 503 | GAGGTTATCTTTTTACCACAGTTGCAC | 45 |
| 504 | CCAGCTTTACAGTGAATTGCTGC | 45 |
| 505 | AGATCTTGACCAATGGCTAAGTGAAG | 45 |
| 506 | TCTAGGGCCTCTTGTGCCTTT | 45 |
| 507 | TCCAGAGGCTAGCAGTTCAACT | 45 |
| 508 | CAGACTTTTGTAATTTGTGTATGCTGATCT | 45 |
| 509 | CACCCCTCGCAGCACCCC | 45 |
| 510 | AAGAGGGCGAGGAGGAGC | 45 |
| 511 | AGCGGGAACAGGACTGCT | 45 |
| 512 | GCCCTGCACCTCCTGGAT | 45 |
| 513 | TGCAGATGGGGGCAAGGT | 45 |
| 514 | GCACCTGGGAGGGCAGAA | 45 |
| 515 | AC CAGTAGGCAACCGTGAAGA | 61 |
| 516 | AGATTACGAAGGTATTGGTTTAGACAGAAA | 61 |
| 517 | AAAATGAAAAACCTTACAGGAAATGGCT | 61 |
| 518 | AACAGTCCATTGGCAGTTGAGAA | 61 |
| 519 | ATGCCCAATTTGATGTTGATGGC | 61 |
| 520 | CCAAAGGGATTTTGTAGATGTTTCTCCA | 61 |
| 521 | CGCATTTCCACAGCTACACCA | 306 |
| 522 | GCATTTGACTTTACCTTATCAATGTCTCGA | 306 |
| 523 | GCTATATCTGAACAAAAATTCCGTGGTT | 204 |
| 524 | AGGGTTCTCCTCCATGGTAGATAC | 204 |
| 525 | TTCCCATTATTATAGAGATGATTGTTGAAT | 511 |
| 526 | CCAGATACTAGAGTGTCTGTGTAATC | 511 |
| 527 | CATTGGCATGGGGAAATATAAACTTGT | 204 |
| 528 | AATAGGGTTCAGCAAATCTTCTAATCCA | 204 |
| 529 | TGGCTTTGAATCTTTGGCCAGT | 61 |
| 530 | ACATAAGAGAGAAGGTTTGACTGCC | 61 |
| 531 | TTTTGGATTACAGGTGCTTATGAATCAAC | 511 |
| 532 | TCTTTGACGGCAATATTACGAAATCCT | 511 |
| 533 | GTCATATAGGAAGTAGAGGAAAGTATTC | 511 |
| 534 | TTAACAGGAAATTTCTAAATGTGACATG | 511 |
| 535 | TCTGTCACCAGGTACAGTAAGTAGG | 204 |
| 536 | AAAGGAATAGTTGCATGTACAGAGTCA | 204 |
| 537 | CGAGATCGTGCTGTTCCACTC | 511 |
| 538 | GTGTAAGATTGAGAAATCTCCAAGGATCT | 511 |
| 539 | ACACAAAGAGAATCTAGTGATTACAGTGT | 511 |
| 540 | ACCAAGGCACAAGATCAAAATCATTC | 511 |
| 541 | TGGGAAGTAATAAAAGATCACCTTCAGAA | 511 |
| 542 | TGAAAGGATTCCACTGAAAGTTTTCTGA | 511 |
| 543 | TTTGATGAGGTGAAGTCCATTGCT | 61 |
| 544 | GTCTCTCTTTGCTGTGCCATCC | 61 |
| 545 | TCTTCCTTATTTTGCCTATGAGGGTAC | 61 |
| 546 | TTGAAGCCATACCTGTTTTCCCAA | 61 |

TABLE 3
Oligonucleotides used in Example 3

| | SEQ | |
|---|---|---|
| | ID | |
| Sequence Name | NO | Sequence (5′-3′) |
| 16-365 | 547 | TCAGACGTGTGCTCTTC |
| | | CGAT*C*T |
| Forward target-specific | 548 | TCAGACGTGTGCTCTTC |
| primers (target specific | | CGATCTAGCAGGATCGG |
| sequence denoted by XXXs) | | TATGG-CXXXXXXXXXX |
| | | XXXXXXXXXX |
| Reverse target-specific | 549 | TCAGACGTGTGCTCTTC |
| primers (target specific | | CGATCTXXXXXXXXXXX |
| sequence denoted by XXXs) | | XXXXXXXXX |
| 18-190 | 550 | AATGATACGGCGACCAC |
| | | CGAGATCTACACTCTTT |
| | | CCCTACACGACGCTCTT |
| | | CCGATCTAGCAGGATCG |
| | | GTATGGC |
| 17-1195 | 551 | CAAGCAGAAGACGGCAT |
| | | ACGAGATTTCTGAATGT |
| | | GACTGGAGTTCAGACGT |
| | | GTGCTCTTCCGATCT |
| 17-1196 | 552 | CAAGCAGAAGACGGCAT |
| | | ACGAGATACGAATTCGT |
| | | GACTGGAGTTCAGACGT |
| | | GTGCTCTTCCGATCT |

### Example 1

### Addition of a Partially Double-Stranded Polynucleotide Duplex With a 5′ Overhang Containing a riboU Stretch at Different Concentrations to Determine the Effect on Multiplex PCR Amplification

Materials

Inhibitor oligonucleotide (Table 1, 18-57)

Inhibitor oligonucleotide (Table 1, 18-58)

Accel-Amplicon 56G Oncology Panel (Swift Biosciences, cat #AL-56248)

10 ng/μl Human genomic DNA (Coriell Institute, NA12878)

Low TE buffer (Teknova cat #TO227)

Methods

An Accel-Amplicon 56G library was made following the manufacturers protocol with the following changes. A partially double stranded polynucleotide duplex with a 5′ overhang and a 4 riboU stretch at each end was made by combining oligonucleotides 18-57 and 18-58 at equimolar concentrations in low TE buffer and is referred to as the polymerase inhibitor in this example. The multiplex PCR reaction was set up by first adding the amount of polymerase inhibitor to the polymerase enzyme that would make a final concentration of 0 μM, 0.1 μM, 0.5 μM, 1 μM, 5 μM, or 10 μM in the 30 μl multiplex PCR reaction. Reactions were set-up on ice and at room temperature for each concentration. The additional reagents were then added to the reactions either on ice or at room temperature such that the 30 μl reaction volume consisted of 24 μl polymerase enzyme plus polymerase inhibitor, 2 μl target-specific primers, 3 μl universal primer, and 1 μl of human genomic DNA. For room temperature set-up, the reaction was incubated at room temperature for 30 minutes before cycling. For ice set-up, the reaction was immediately placed in the thermocycler for amplification. PCR amplification, adapter ligation, and library quantification were performed as described by the manufacturer. Libraries were sequenced on a MiniSeq (Illumina) with paired end reads of 151 bases.

Results

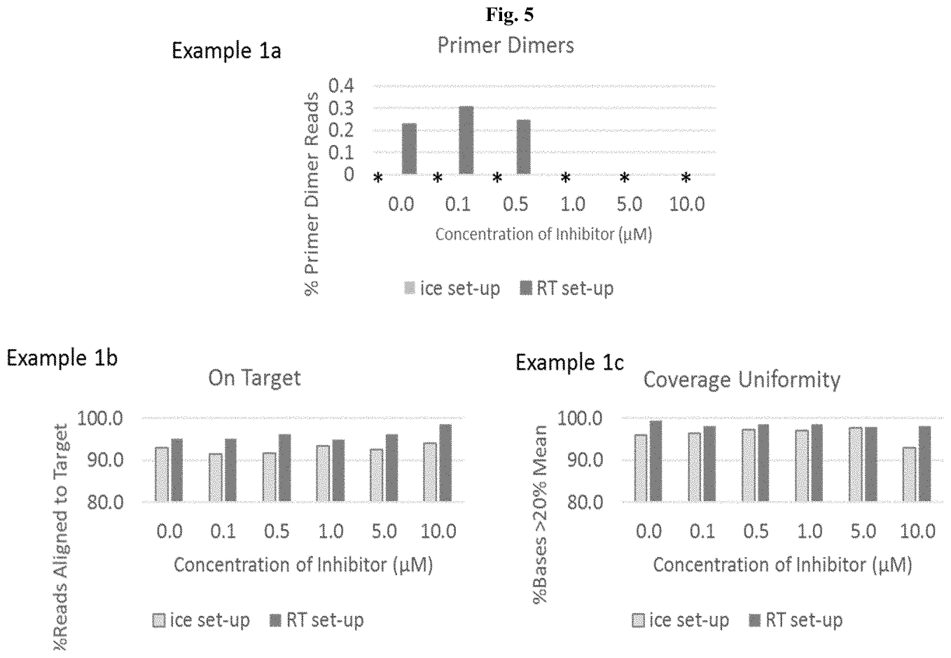

Prior to data analysis, sequence-specific primer trimming was performed from the 5′ end of both read 1 and read 2 to remove synthetic primer sequences. Reads were aligned to the human genome and to the target regions. Primer dimers were defined as reads with an insert size of less than 35 bases. No primer dimer formation was detected with an on-ice set-up and addition of the polymerase inhibitor eliminated detectable primer dimers with a room temperature set-up when added at 1 μM or greater (FIG. 5). Asterisks on Example 1a Table indicate primer dimers less than 0.1% but the actual value is not reported, as Illumina software does not report frequencies of reads with an insert size of less than 35 bases when the frequency is below 0.1%. On-target reads were defined as reads that map to the target regions. Percent of on target reads was high, greater than 90%, for all conditions tested (FIG. 5). Coverage uniformity was defined as the number of target bases higher than 20% of the mean per base coverage and describes how evenly the 263 target amplicons were represented in the final 56G panel library. Coverage uniformity was high, greater than 90%, for all conditions tested (FIG. 5). A slight decrease in coverage uniformity was observed in the presence of 10 μM polymerase inhibitor.

Conclusions

Addition of a partially double-stranded polynucleotide duplex with a 5′ overhang containing a 4 riboU stretch decreased primer dimer amplification when added at greater than or equal to 1 μM in a multiplex PCR with a non-hotstart polymerase set up at room temperature. Addition of this molecule did not have a notable effect on other sequencing metrics used to evaluate multiplex PCR quality, on target and coverage uniformity, when used at less than 10 μM.

### Example 2

### Addition of a Partially Double-Stranded Polynucleotide Duplex With a 5′ Overhang Containing a riboU Stretch Reduced Primer Dimer Amplification and Increased Polymerase Specificity in a Multiplex PCR Reaction With a Non-Hotstart DNA Polymerase

Materials

Inhibitor oligonucleotide (Table 1, 18-57)

Inhibitor oligonucleotide (Table 1, 18-58)

Accel-Amplicon Custom NGS Panel (Swift Biosciences)

10 ng/μl Human genomic DNA (Coriell Institute, NA12878)

Low TE buffer (Teknova cat #TO227)

544 target-specific primers (Table 2)

Methods

An Accel-Amplicon NGS library was made following the manufacturers protocol with the following changes. For the multiplex PCR reaction, a custom set of target-specific primer pairs was used consisting of a mix of 544 target-specific primers present at different concentrations as indicated in Table 2. Primers listed in Table 2 have a 5′ tail of the following sequence TCAGACGTGTGCTCTTCCGATCT (SEQ ID NO: 553). This panel was not fully optimized for amplicon performance and therefore made it possible to observe changes in nonspecific priming by the polymerase. A partially double stranded polynucleotide duplex with a 5′ overhang and a 4 riboU stretch at each end was made by combining oligonucleotides 18-57 and 18-58 at 30 μM each and is referred to as the polymerase inhibitor in this example. The multiplex PCR reaction was set up by first adding 5 μl of the polymerase inhibitor for a final concentration of 5 μM or 5 μl of low TE buffer to polymerase at room temperature or on ice. The additional reagents were then added to the reactions either at room temperature or on ice such that the 30 μl reaction volume consisted of 20 μl polymerase plus polymerase inhibitor or low TE buffer, 2 μl target-specific primer pairs, 3 μl universal primer, 1 μl of human genomic DNA, and 4 μl low TE buffer. For room temperature set-up, the reaction was incubated at room temperature for 30 minutes before cycling. For ice set-up, the reaction was immediately placed in the thermocycler for amplification. The following cycling program was run on all reaction mixes: 30 seconds at 98° C. followed by 4 cycles of 10 seconds at 98° C., 5 minutes at 63° C., and 1 minute at 65° C., then 22 cycles of 10 seconds at 98° C. and 1 minute at 64° C., and completed with 1 minute at 65° C. Reaction purification, adapter ligation, and library quantification was performed as described by the manufacturer. Libraries were sequenced on a MiniSeq (Illumina) with paired end reads of 151 bases.

Results

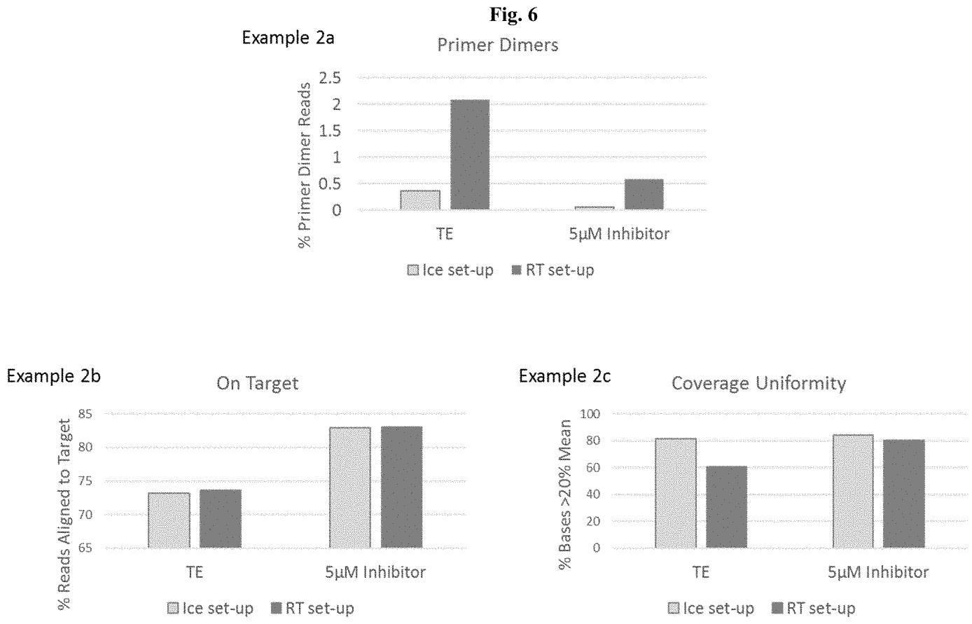

All libraries were prepared in duplicate and data shown are an average of the two libraries. Prior to data analysis, sequence-specific primer trimming was performed from the 5′ end of both read 1 and read 2 to remove synthetic primer sequences. Reads were aligned to the human genome and to the target regions. Primer dimers were defined as reads with an insert size of less than 35 bases. Primer dimer formation increased by greater than 5-fold with a room temperature set-up compared to ice (FIG. 6). The addition of the polymerase inhibitor reduced primer dimer formation with both a room temperature and ice set-up such that the room-temperature set-up in the polymerase inhibitor displayed close to the same percent primer dimer reads as the ice set up with low TE buffer (FIG. 6). Reduced polymerase specificity during PCR can result in off target products as well as a reduction in the intended target amplification. The percent of on target reads as well as the coverage uniformity of the intended targets were assessed in order to evaluate polymerase specificity. On target reads were defined as reads that map to the target regions. Percent of on target reads increased by roughly 10% in the presence of the polymerase inhibitor with both ice and room temperature set-up (FIG. 6). Coverage uniformity was defined as the number of target bases higher than 20% of the mean per base coverage and describes how evenly the 274 amplicons were represented in the final library. Coverage uniformity was reduced by 20% with room temperature compared to ice set-up and this was rescued by the addition of the polymerase inhibitor (FIG. 6).

Conclusions

Addition of a partially double-stranded polynucleotide duplex with a 5′ overhang containing a 4 riboU stretch decreased primer dimer amplification and increased polymerase specificity in a multiplex PCR used to create a targeted NGS library. These advantages were most evident with a room temperature set-up but were also observed when the reaction was set-up on ice.

### Example 3

### Addition of a Partially Double-Stranded Polynucleotide Duplex With a 5′ Overhang Containing a riboU Stretch Reduced Unintended Products in a Multiplex PCR Reaction With a Hotstart DNA Polymerase

Materials

Inhibitor oligonucleotide (Table 1, 18-57)

Inhibitor oligonucleotide (Table 1, 18-58)

Universal primer (Table 3, 16-365)

Q5® Hot Start High-Fidelity 2× Master Mix (NEB, cat #M0494)

10 ng/μl genomic DNA

Low TE buffer (Teknova cat #T0227)

1244 forward target-specific primers (Table 3)

1244 reverse target-specific primers (Table 3)

P5 primer consisting of full-length Illumina P5 adapter sequence and a 3′ tag (Table 3, 18-190)

P7 indexing primers consisting of full-length Illumina P7 adapter sequence (Table 3, 17-1195 and 17-1196)

SPRIselect reagent (Beckman Coulter, B23318)

20% PEG-8000/2.5M NaCl solution.

Methods

Genomic DNA was diluted in low TE buffer. 1244 target-specific forward primers and 1244 target-specific reverse primers, targeting hotspot SNPs found throughout the genome were designed with a melting temperature between 62.5° C. and 68.0° C. and with an amplicon size from 116 to 211 base pairs. These 2488 target-specific primers were combined at 60 nM each. For each amplicon both the forward and reverse target-specific primers contained the following 23 base pair universal sequence, TCAGACGTGTGCTCTTCCGATCT (SEQ ID NO: 554), at the 5′ end. The forward target-specific primer also contained the following 17 base pair sequence, AGCAGGATCGGTATGGC (SEQ ID NO: 555), between the 23 base pair universal sequence and the target-specific sequence. A partially double stranded polynucleotide duplex with a 5′ overhang and a 4 riboU stretch at each end was made by combining oligonucleotides 18-57 and 18-58 at 30 μM each and is referred to as the polymerase inhibitor in this example. A first multiplex PCR reaction for target selection and amplification was performed in 30 μl. The reaction was set up by first adding 5 μl of the polymerase inhibitor for a final concentration of 5 μM or 5 μl of low TE buffer to 15 μl of Q5 Hot Start High-Fidelity 2× Master Mix (2000 U/mL) on ice. The additional reagents were then added to the reactions on ice such that the 30 μl reaction volume consisted of 20 μl Q5 Hot Start High-Fidelity 2× Master Mix plus polymerase inhibitor or low TE buffer, 3 μl 100 μM universal primer 16-365, 2 μl target-specific primer mix, 1 μl of genomic DNA, and 4 μl low TE buffer. The following cycling program was run on all reaction mixes: 30 seconds at 98° C. followed by 4 cycles of 10 seconds at 98° C. and 6 minutes at 66° C., then 18 cycles of 10 seconds at 98° C., 15 seconds at 60° C., and 1 minute at 66° C., and completed with 1 minute at 65° C. A purification was performed with 30 μl SPRIselect beads (1.0× ratio) and the beads were resuspended in 30 μl of a second reaction mix containing 15 μl Q5 Hot Start High-Fidelity 2× Master Mix, 2.5 μl 6 uM P5 primer 18-190, and 2.5 ul 6 μM P7 indexing primer 17-1195 or 17-1196. The following cycling program was run on all reaction mixes: 45 seconds at 98° C. followed by 8 cycles of 10 seconds at 98° C., 15 seconds at 60° C., and 1 minute at 66° C. to index and add full-length adapters to the amplicons. The reaction was purified with 26 μl of 20% PEG-8000/2.5M NaCl solution (0.85× ratio) and the DNA was eluted in 20 μl low TE Buffer. Library was quantified by qPCR and sequenced on a Mini Seq (Illumina) with paired end reads of 151 bases.

Results

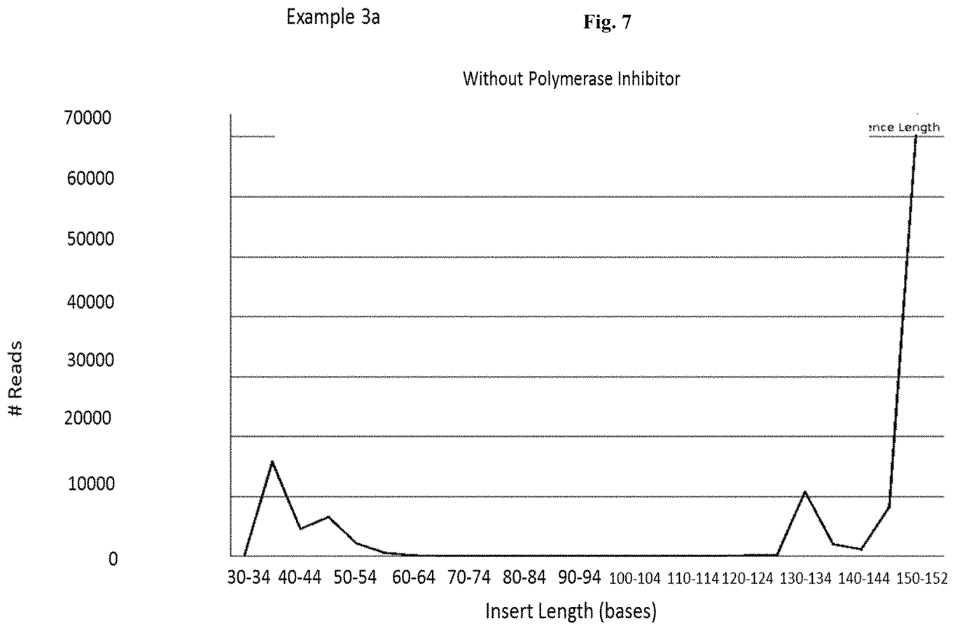

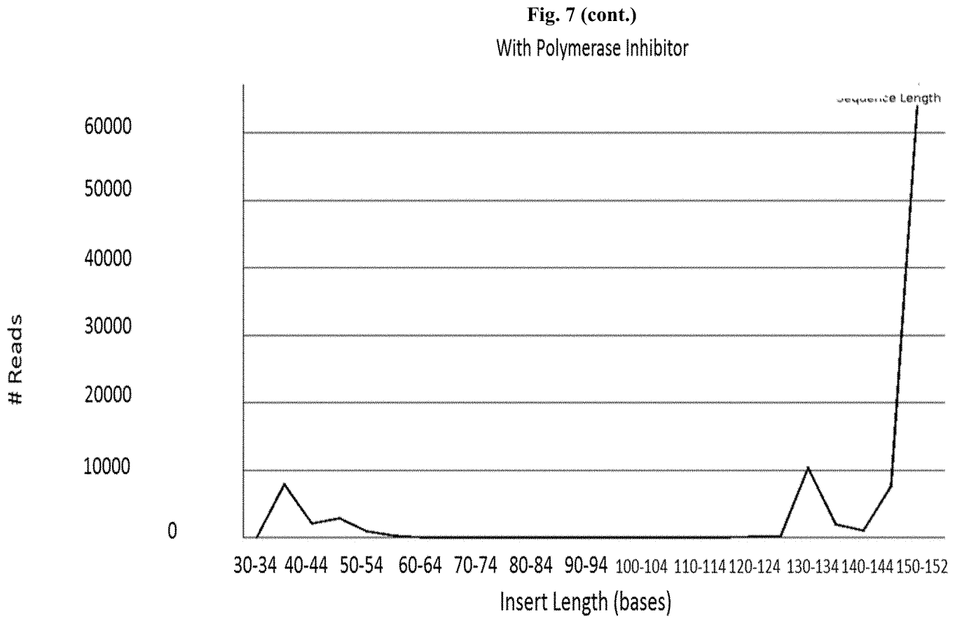

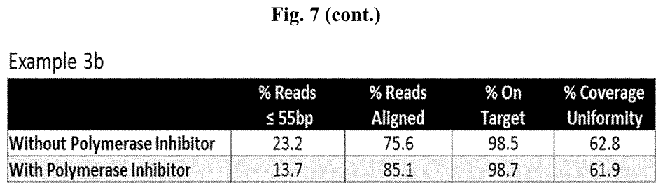

Adapter trimming was performed, and reads were aligned to the reference genome and to the target regions. Intended amplicons had a minimum insert length of 133 bp, a 116 bp minimal amplicon size plus a 17 bp tag from the forward primer. Therefore, any sequences shorter than 133 bp are unintended products from primer dimer formation and/or off-target priming. Read length was assessed in the presence or absence of the polymerase inhibitor and the presence of short reads, less than 55 bp, was reduced by 9.5% in the presence of the polymerase inhibitor (FIG. 7). Short reads, especially those that result from primer dimers that do not align to unique genomic positions, are difficult to map using standard aligners. In the presence of the polymerase inhibitor the percent of reads aligned to the reference genome was increased by 9.5% depicting the increase in usable data in the presence of the polymerase inhibitor. Coverage uniformity, defined as the number of target bases higher than 20% of the mean per base coverage, and percent of mapped reads that are on-target were not affected by the polymerase inhibitor.

Conclusions

Addition of a partially double-stranded polynucleotide duplex with a 5′ overhang containing a 4 riboU stretch decreased the presence of short, unwanted reads in a multiplex PCR with a hotstart DNA polymerase used to create a targeted NGS library.

It should be understood that the foregoing description provides embodiments of the present invention which can be varied and combined without departing from the spirit of this disclosure. To the extent that the different aspects disclosed can be combined, such combinations are disclosed herein.

What is claimed is:

1-183. (canceled)

184. A polymerase inhibitor comprising a synthetic nucleic acid molecule comprising:
a first oligonucleotide comprising a first complementary region; and
a second oligonucleotide comprising a second complementary region and a first single-stranded region positioned 5′ to the second complementary region,
wherein the first complementary region is sufficiently complementary to the second complementary region to form a double-stranded region at a temperature below a melting temperature of the polymerase inhibitor,
wherein the first oligonucleotide further comprises a second single-stranded region positioned 5′ to the first complementary region,
wherein the first single-stranded region comprises a first replication blocking sequence or the first oligonucleotide further comprises a first extension blocking group at a 3′ end of the first oligonucleotide, and
wherein the second single-stranded region comprises a second replication blocking sequence or the second oligonucleotide further comprises a second extension blocking group at a 3′ end of the second oligonucleotide.

185. The polymerase inhibitor of claim 184, wherein first complementary region and second complementary region are from about 6 to about 100 nucleotides.

186-187. (canceled)

188. The polymerase inhibitor of claim 184, wherein the first complementary region is sufficiently complementary to the second complementary region to form a double-stranded region below the melting temperature, and wherein the melting temperature is selected from the group consisting of below 90° C., 80° C., 70° C., 60° C., and 50° C.

189-191. (canceled)

192. The polymerase inhibitor of claim 184, wherein the first complementary region comprises a homopolymer sequence or a heteropolymeric sequence comprising a dinucleotide sequence.

193-199. (canceled)

200. The polymerase inhibitor of claim 184, wherein the first single-stranded region comprises the first replication blocking sequence and the second single-stranded region comprises the second replication blocking sequence.

201. The polymerase inhibitor of claim 184, wherein the first oligonucleotide further comprises the first extension blocking group and the second oligonucleotide further comprises the second extension blocking group.

202. The polymerase inhibitor of claim 184, wherein the first single-stranded region comprises the first replication blocking sequence and the second oligonucleotide further comprises the second extension blocking group.

203. The polymerase inhibitor of claim 184, wherein the second single-stranded region comprises the second replication blocking sequence and the first oligonucleotide further comprises the first extension blocking group.

204. The polymerase inhibitor of claim 184, wherein the first single-stranded region comprises the first replication blocking sequence, the second single-stranded region comprises the second replication blocking sequence, the first oligonucleotide further comprises the first extension blocking group, and the second oligonucleotide further comprises the second extension blocking group.

205. The polymerase inhibitor of claim 200, wherein the first replication blocking sequence and the second replication blocking sequence are each selected from the group consisting of a stable abasic site, a carbon spacer, at least one 2′-O methyl nucleotide, a deoxyuridine base, a deoxyinosine base, and from about 2 to about 50 consecutive ribonucleotide bases.

206. The polymerase inhibitor of claim 201, wherein the first extension blocking group and the second extension blocking group are each selected from the group consisting of a 3′ phosphate, a carbon spacer, and a dideoxynucleotide.

207. The polymerase inhibitor of claim 202, wherein the first replication blocking sequence is selected from the group consisting of a stable abasic site, a carbon spacer, at least one 2′-O methyl nucleotide, a deoxyuridine base, a deoxyinosine base, and from about 2 to about 50 consecutive ribonucleotide bases, and wherein the second extension blocking group is selected from the group consisting of a 3′ phosphate, a carbon spacer, and a dideoxynucleotide.

208. The polymerase inhibitor of claim 203, wherein the second replication blocking sequence is selected from the group consisting of a stable abasic site, a carbon spacer, at least one 2′-O methyl nucleotide, a deoxyuridine base, a deoxyinosine base, and from about 2 to about 50 consecutive ribonucleotide bases, and wherein the first extension blocking group is selected from the group consisting of a 3′ phosphate, a carbon spacer, and a dideoxynucleotide.

209. The polymerase inhibitor of claim 204, wherein the first replication blocking sequence and the second replication blocking sequence are each selected from the group consisting of a stable abasic site, a carbon spacer, at least one 2′-O methyl nucleotide, a deoxyuridine base, a deoxyinosine base, and from about 2 to about 50 consecutive ribonucleotide bases, and wherein wherein the first extension blocking group and the second extension blocking group are each selected from the group consisting of a 3′ phosphate, a carbon spacer, and a dideoxynucleotide.

210. The polymerase inhibitor of claim 201, wherein the first oligonucleotide further comprises a first nuclease resistant linkage at the 3′ end of the first oligonucleotide.

211. The polymerase inhibitor of claim 201, wherein the second oligonucleotide further comprises a second nuclease resistant linkage at the 3′ end of the second oligonucleotide.

212. The polymerase inhibitor of claim 201, wherein the first oligonucleotide further comprises a first nuclease resistant linkage at the 3′ end of the first oligonucleotide, and wherein the second oligonucleotide further comprises a second nuclease resistant linkage at the 3′ end of the second oligonucleotide.

213. The polymerase inhibitor of claim 206, wherein the first oligonucleotide further comprises a first nuclease resistant linkage at the 3′ end of the first oligonucleotide.

214. The polymerase inhibitor of claim 206, wherein the second oligonucleotide further comprises a second nuclease resistant linkage at the 3′ end of the second oligonucleotide.

215. The polymerase inhibitor of claim 206, wherein the first oligonucleotide further comprises a first nuclease resistant linkage at the 3′ end of the first oligonucleotide, and wherein the second oligonucleotide further comprises a second nuclease resistant linkage at the 3′ end of the second oligonucleotide.

216-449. (canceled)


SEQUENCE LISTING (577 sequences)

SEQ ID NO: 1 - DNA, 24 bp, Artificial Sequence
  Description of Artificial Sequence Synthetic oligonucleotide
  Description of Combined DNA/RNA Molecule Synthetic oligonucleotide
agtcuuuuggttttttttttttgg

SEQ ID NO: 2 - DNA, 24 bp, Artificial Sequence
  Description of Artificial Sequence Synthetic oligonucleotide
  Description of Combined DNA/RNA Molecule Synthetic oligonucleotide
ccaaaaaaaaaaaaccuuuuctga

SEQ ID NO: 3 - DNA, 18 bp, Artificial Sequence
  Description of Artificial Sequence Synthetic primer
catcgtggggcctggtgg

SEQ ID NO: 4 - DNA, 18 bp, Artificial Sequence
  Description of Artificial Sequence Synthetic primer
tggctacaaggagcggcc

SEQ ID NO: 5 - DNA, 22 bp, Artificial Sequence
  Description of Artificial Sequence Synthetic primer
cagagaagttgttgaggggagc

SEQ ID NO: 6 - DNA, 18 bp, Artificial Sequence
  Description of Artificial Sequence Synthetic primer
catcaagacctggcggcc

SEQ ID NO: 7 - DNA, 25 bp, Artificial Sequence
  Description of Artificial Sequence Synthetic primer
catcattcttgaggaggaagtagcg

SEQ ID NO: 8 - DNA, 19 bp, Artificial Sequence
  Description of Artificial Sequence Synthetic primer
aggcacatctgtcctggca

SEQ ID NO: 9 - DNA, 30 bp, Artificial Sequence
  Description of Artificial Sequence Synthetic primer
tctagtttttagaagtacccagatttgaca

SEQ ID NO: 10 - DNA, 22 bp, Artificial Sequence
  Description of Artificial Sequence Synthetic primer
ttgtgcctccactgtccaaaaa

SEQ ID NO: 11 - DNA, 30 bp, Artificial Sequence
  Description of Artificial Sequence Synthetic primer
actcattttgtgtaggaaaggtacaatgat

SEQ ID NO: 12 - DNA, 26 bp, Artificial Sequence
  Description of Artificial Sequence Synthetic primer
tccacagatttatatcatccaggcct

SEQ ID NO: 13 - DNA, 29 bp, Artificial Sequence
  Description of Artificial Sequence Synthetic primer
gtagttctgttaaagttcatggcttttgt

SEQ ID NO: 14 - DNA, 18 bp, Artificial Sequence
  Description of Artificial Sequence Synthetic primer
tttcagtgcacgcagggc

SEQ ID NO: 15 - DNA, 23 bp, Artificial Sequence
  Description of Artificial Sequence Synthetic primer
cgagagctggagttggatgaatt

SEQ ID NO: 16 - DNA, 20 bp, Artificial Sequence
  Description of Artificial Sequence Synthetic primer
gccagagggaacaaagtcgg

SEQ ID NO: 17 - DNA, 25 bp, Artificial Sequence
  Description of Artificial Sequence Synthetic primer
gtggagaagaacatgatatgtgggt

SEQ ID NO: 18 - DNA, 25 bp, Artificial Sequence
  Description of Artificial Sequence Synthetic primer
tgtctaagtttttcaagcacagggt

SEQ ID NO: 19 - DNA, 23 bp, Artificial Sequence
  Description of Artificial Sequence Synthetic primer
tggaccacacaggagaatatgga

SEQ ID NO: 20 - DNA, 23 bp, Artificial Sequence
  Description of Artificial Sequence Synthetic primer
gctcaccttaacaagctgtctcc

SEQ ID NO: 21 - DNA, 30 bp, Artificial Sequence
  Description of Artificial Sequence Synthetic primer
ttctttaagtgcaaatagtgtatctgacct

SEQ ID NO: 22 - DNA, 25 bp, Artificial Sequence
  Description of Artificial Sequence Synthetic primer
atttacacctcctgctaagcgaaat

SEQ ID NO: 23 - DNA, 22 bp, Artificial Sequence
  Description of Artificial Sequence Synthetic primer
gattgtgtaggttccgatggca

SEQ ID NO: 24 - DNA, 30 bp, Artificial Sequence
  Description of Artificial Sequence Synthetic primer
aaagacaaaatcccaaataaagcagaaaga

SEQ ID NO: 25 - DNA, 29 bp, Artificial Sequence
  Description of Artificial Sequence Synthetic primer
tggaatcagtgatttcagattgtttgttt

SEQ ID NO: 26 - DNA, 29 bp, Artificial Sequence
  Description of Artificial Sequence Synthetic primer
agatctatatgtacaagttctgctgactg

SEQ ID NO: 27 - DNA, 27 bp, Artificial Sequence
  Description of Artificial Sequence Synthetic primer
aaagtagctgaacgtgtcttaatgaga

SEQ ID NO: 28 - DNA, 20 bp, Artificial Sequence
  Description of Artificial Sequence Synthetic primer
tcctgggaaaagtcggctga

SEQ ID NO: 29 - DNA, 21 bp, Artificial Sequence
  Description of Artificial Sequence Synthetic primer
gcaggccatagaccccaaaaa

SEQ ID NO: 30 - DNA, 26 bp, Artificial Sequence
  Description of Artificial Sequence Synthetic primer
ccctacttaaagtatgttggcaggtt

SEQ ID NO: 31 - DNA, 28 bp, Artificial Sequence
  Description of Artificial Sequence Synthetic primer
tctttacatggcttttggtcttctaagt

SEQ ID NO: 32 - DNA, 21 bp, Artificial Sequence
  Description of Artificial Sequence Synthetic primer
ccattctggcacgctttggaa

SEQ ID NO: 33 - DNA, 28 bp, Artificial Sequence
  Description of Artificial Sequence Synthetic primer
ctcactatgaaaaactgtaaagctgcaa

SEQ ID NO: 34 - DNA, 26 bp, Artificial Sequence
  Description of Artificial Sequence Synthetic primer
ttattaagatgcagctactacccagc

SEQ ID NO: 35 - DNA, 30 bp, Artificial Sequence
  Description of Artificial Sequence Synthetic primer
tccatgaatctatttaacgattaccctgat

SEQ ID NO: 36 - DNA, 25 bp, Artificial Sequence
  Description of Artificial Sequence Synthetic primer
gaggcctcttatactgccaaatcaa

SEQ ID NO: 37 - DNA, 21 bp, Artificial Sequence
  Description of Artificial Sequence Synthetic primer
gccatttgaccgtggagaagt

SEQ ID NO: 38 - DNA, 22 bp, Artificial Sequence
  Description of Artificial Sequence Synthetic primer
actttggctctctccaggttcg

SEQ ID NO: 39 - DNA, 21 bp, Artificial Sequence
  Description of Artificial Sequence Synthetic primer
ttgactacagcatgctcctgc

SEQ ID NO: 40 - DNA, 24 bp, Artificial Sequence
  Description of Artificial Sequence Synthetic primer
tcctccacctccattagatttcca

SEQ ID NO: 41 - DNA, 29 bp, Artificial Sequence
  Description of Artificial Sequence Synthetic primer
tttttcagtggaggttaacattcatcaag

SEQ ID NO: 42 - DNA, 25 bp, Artificial Sequence
  Description of Artificial Sequence Synthetic primer
gaaactatgtccagtctttgtggct

SEQ ID NO: 43 - DNA, 22 bp, Artificial Sequence
  Description of Artificial Sequence Synthetic primer
agttcgatcagcagctgttacc

SEQ ID NO: 44 - DNA, 21 bp, Artificial Sequence
  Description of Artificial Sequence Synthetic primer
aaggctgtagataggccagca

SEQ ID NO: 45 - DNA, 29 bp, Artificial Sequence
  Description of Artificial Sequence Synthetic primer
gggagatttataagatgacaacagatcca

SEQ ID NO: 46 - DNA, 22 bp, Artificial Sequence
  Description of Artificial Sequence Synthetic primer
agtcatgacccacagcaaacag

SEQ ID NO: 47 - DNA, 18 bp, Artificial Sequence
  Description of Artificial Sequence Synthetic primer
ttggagccgcagcctctc

SEQ ID NO: 48 - DNA, 20 bp, Artificial Sequence
  Description of Artificial Sequence Synthetic primer
tggaagaagggaagcggtga

SEQ ID NO: 49 - DNA, 18 bp, Artificial Sequence
  Description of Artificial Sequence Synthetic primer
actccagcccgctccagc

SEQ ID NO: 50 - DNA, 18 bp, Artificial Sequence
  Description of Artificial Sequence Synthetic primer
ccctcgcaagtcagggga

SEQ ID NO: 51 - DNA, 18 bp, Artificial Sequence
  Description of Artificial Sequence Synthetic primer
cctgggcagaggtgaggg

SEQ ID NO: 52 - DNA, 22 bp, Artificial Sequence
  Description of Artificial Sequence Synthetic primer
ctgcgtaaattccaaggggtgt

SEQ ID NO: 53 - DNA, 22 bp, Artificial Sequence
  Description of Artificial Sequence Synthetic primer
caggctttgtggatttgaccct

SEQ ID NO: 54 - DNA, 23 bp, Artificial Sequence
  Description of Artificial Sequence Synthetic primer
ctgtccaagagcaagttaggagc

SEQ ID NO: 55 - DNA, 18 bp, Artificial Sequence
  Description of Artificial Sequence Synthetic primer
ggagcacccagggaagct

SEQ ID NO: 56 - DNA, 23 bp, Artificial Sequence
  Description of Artificial Sequence Synthetic primer
tctgaagcttcaccgagagatga

SEQ ID NO: 57 - DNA, 22 bp, Artificial Sequence
  Description of Artificial Sequence Synthetic primer
ccagggaaaatgtgtagagggc

SEQ ID NO: 58 - DNA, 28 bp, Artificial Sequence
  Description of Artificial Sequence Synthetic primer
tgtgttatcaactcaccagaattaagca

SEQ ID NO: 59 - DNA, 27 bp, Artificial Sequence
  Description of Artificial Sequence Synthetic primer
ccagtggatttttatgaatgtgaaccc

SEQ ID NO: 60 - DNA, 22 bp, Artificial Sequence
  Description of Artificial Sequence Synthetic primer
cagcatgtcgaagatctccacc

SEQ ID NO: 61 - DNA, 21 bp, Artificial Sequence
  Description of Artificial Sequence Synthetic primer
cagggagaggagtttgtgtgc

SEQ ID NO: 62 - DNA, 23 bp, Artificial Sequence
  Description of Artificial Sequence Synthetic primer
gacattatgcctttggagtgggt

SEQ ID NO: 63 - DNA, 24 bp, Artificial Sequence
  Description of Artificial Sequence Synthetic primer
tcctagacctcatcctctttgagc

SEQ ID NO: 64 - DNA, 23 bp, Artificial Sequence
  Description of Artificial Sequence Synthetic primer
tgtctgtgatcttgtccaggact

SEQ ID NO: 65 - DNA, 18 bp, Artificial Sequence
  Description of Artificial Sequence Synthetic primer
ggcctgaccctgcagcag

SEQ ID NO: 66 - DNA, 18 bp, Artificial Sequence
  Description of Artificial Sequence Synthetic primer
cccggccagctgtcacat

SEQ ID NO: 67 - DNA, 18 bp, Artificial Sequence
  Description of Artificial Sequence Synthetic primer
tcagcactcctggggctc

SEQ ID NO: 68 - DNA, 19 bp, Artificial Sequence
  Description of Artificial Sequence Synthetic primer
tccagcatctccagcagca

SEQ ID NO: 69 - DNA, 18 bp, Artificial Sequence
  Description of Artificial Sequence Synthetic primer
tgcaagaacgtggtgccc

SEQ ID NO: 70 - DNA, 20 bp, Artificial Sequence
  Description of Artificial Sequence Synthetic primer
ccaagtggctttggtccgtc

SEQ ID NO: 71 - DNA, 18 bp, Artificial Sequence
  Description of Artificial Sequence Synthetic primer
atgcgcccactagccgtg

SEQ ID NO: 72 - DNA, 19 bp, Artificial Sequence
  Description of Artificial Sequence Synthetic primer
ggaaaccctctgcctcccc

SEQ ID NO: 73 - DNA, 22 bp, Artificial Sequence
  Description of Artificial Sequence Synthetic primer
gggctctacttcatcgcattcc

SEQ ID NO: 74 - DNA, 23 bp, Artificial Sequence
  Description of Artificial Sequence Synthetic primer
gctaaagtggtgcatgatgaggg

SEQ ID NO: 75 - DNA, 19 bp, Artificial Sequence
  Description of Artificial Sequence Synthetic primer
gaaccctattggtgttact

SEQ ID NO: 76 - DNA, 20 bp, Artificial Sequence
  Description of Artificial Sequence Synthetic primer
agtcaaactccaactctaag

SEQ ID NO: 77 - DNA, 24 bp, Artificial Sequence
  Description of Artificial Sequence Synthetic primer
tccccgaaattctacccaaattgc

SEQ ID NO: 78 - DNA, 30 bp, Artificial Sequence
  Description of Artificial Sequence Synthetic primer
gtggactttctgagagaaaacaatttaagt

SEQ ID NO: 79 - DNA, 24 bp, Artificial Sequence
  Description of Artificial Sequence Synthetic primer
caaccttttgaacagcatgcaaga

SEQ ID NO: 80 - DNA, 25 bp, Artificial Sequence
  Description of Artificial Sequence Synthetic primer
acctgggctacttcatctctagaat

SEQ ID NO: 81 - DNA, 25 bp, Artificial Sequence
  Description of Artificial Sequence Synthetic primer
tcttttctcaagttggcctgaatca

SEQ ID NO: 82 - DNA, 25 bp, Artificial Sequence
  Description of Artificial Sequence Synthetic primer
tgacgatctccaattcccaaaatga

SEQ ID NO: 83 - DNA, 23 bp, Artificial Sequence
  Description of Artificial Sequence Synthetic primer
cgttcatgtgctggatactgtgt

SEQ ID NO: 84 - DNA, 30 bp, Artificial Sequence
  Description of Artificial Sequence Synthetic primer
aacaccaaaacattttaaacagagaaaacc

SEQ ID NO: 85 - DNA, 24 bp, Artificial Sequence
  Description of Artificial Sequence Synthetic primer
catggtgaaagacgatggacaagt

SEQ ID NO: 86 - DNA, 30 bp, Artificial Sequence
  Description of Artificial Sequence Synthetic primer
agtgtccaaaatctatatgaaacagctttc

SEQ ID NO: 87 - DNA, 27 bp, Artificial Sequence
  Description of Artificial Sequence Synthetic primer
tgatgacattgcatacattcgaaagac

SEQ ID NO: 88 - DNA, 25 bp, Artificial Sequence
  Description of Artificial Sequence Synthetic primer
gcatgctgtttaattgtgtggaaga

SEQ ID NO: 89 - DNA, 22 bp, Artificial Sequence
  Description of Artificial Sequence Synthetic primer
cgacagcatgccaatctcttca

SEQ ID NO: 90 - DNA, 25 bp, Artificial Sequence
  Description of Artificial Sequence Synthetic primer
catgaaatactccaaagcctcttgc

SEQ ID NO: 91 - DNA, 28 bp, Artificial Sequence
  Description of Artificial Sequence Synthetic primer
cctgaaggtattaacatcatttgctcca

SEQ ID NO: 92 - DNA, 22 bp, Artificial Sequence
  Description of Artificial Sequence Synthetic primer
ccagagccaagcatcattgaga

SEQ ID NO: 93 - DNA, 22 bp, Artificial Sequence
  Description of Artificial Sequence Synthetic primer
tcatggtggctggacaacaaaa

SEQ ID NO: 94 - DNA, 20 bp, Artificial Sequence
  Description of Artificial Sequence Synthetic primer
cggtctttgcctgctgagag

SEQ ID NO: 95 - DNA, 18 bp, Artificial Sequence
  Description of Artificial Sequence Synthetic primer
ctctgccaggagccggag

SEQ ID NO: 96 - DNA, 18 bp, Artificial Sequence
  Description of Artificial Sequence Synthetic primer
cctactccgcccagggac

SEQ ID NO: 97 - DNA, 18 bp, Artificial Sequence
  Description of Artificial Sequence Synthetic primer
ggcgctgttggtttcggt

SEQ ID NO: 98 - DNA, 20 bp, Artificial Sequence
  Description of Artificial Sequence Synthetic primer
gtgaacgtgtagctctcggc

SEQ ID NO: 99 - DNA, 22 bp, Artificial Sequence
  Description of Artificial Sequence Synthetic primer
acagtcaaaaggcctctacggt

SEQ ID NO: 100 - DNA, 18 bp, Artificial Sequence
  Description of Artificial Sequence Synthetic primer
atacctgatggggcgggg

SEQ ID NO: 101 - DNA, 22 bp, Artificial Sequence
  Description of Artificial Sequence Synthetic primer
cgctctttggagaaggaatgct

SEQ ID NO: 102 - DNA, 20 bp, Artificial Sequence
  Description of Artificial Sequence Synthetic primer
gtgcccactttgaatcgggt

SEQ ID NO: 103 - DNA, 22 bp, Artificial Sequence
  Description of Artificial Sequence Synthetic primer
ttccaccaaagtcacgctgaat

SEQ ID NO: 104 - DNA, 21 bp, Artificial Sequence
  Description of Artificial Sequence Synthetic primer
gtcaacggtaccaaggctgag

SEQ ID NO: 105 - DNA, 22 bp, Artificial Sequence
  Description of Artificial Sequence Synthetic primer
tggatagagaacgcattgccac

SEQ ID NO: 106 - DNA, 24 bp, Artificial Sequence
  Description of Artificial Sequence Synthetic primer
ttaagctcctcatgtgttcagagc

SEQ ID NO: 107 - DNA, 19 bp, Artificial Sequence
  Description of Artificial Sequence Synthetic primer
gcatcactggccaaggagc

SEQ ID NO: 108 - DNA, 30 bp, Artificial Sequence
  Description of Artificial Sequence Synthetic primer
caacaagcttctaagagatacttacagtgt

SEQ ID NO: 109 - DNA, 27 bp, Artificial Sequence
  Description of Artificial Sequence Synthetic primer
ccagtgttgggatccttctttactaat

SEQ ID NO: 110 - DNA, 28 bp, Artificial Sequence
  Description of Artificial Sequence Synthetic primer
ctttcaataataaagacaccaacagggg

SEQ ID NO: 111 - DNA, 21 bp, Artificial Sequence
  Description of Artificial Sequence Synthetic primer
ttctctgggagggatttggca

SEQ ID NO: 112 - DNA, 28 bp, Artificial Sequence
  Description of Artificial Sequence Synthetic primer
tgtctttgttggatttgatctgaagaca

SEQ ID NO: 113 - DNA, 23 bp, Artificial Sequence
  Description of Artificial Sequence Synthetic primer
agagagactgggttattcctccc

SEQ ID NO: 114 - DNA, 25 bp, Artificial Sequence
  Description of Artificial Sequence Synthetic primer
ttccctttctctccttggtacttct

SEQ ID NO: 115 - DNA, 25 bp, Artificial Sequence
  Description of Artificial Sequence Synthetic primer
catcttctttccttttaggcctccg

SEQ ID NO: 116 - DNA, 22 bp, Artificial Sequence
  Description of Artificial Sequence Synthetic primer
gggcctttttcattttctgggc

SEQ ID NO: 117 - DNA, 21 bp, Artificial Sequence
  Description of Artificial Sequence Synthetic primer
tgtctggctaggttggactgt

SEQ ID NO: 118 - DNA, 20 bp, Artificial Sequence
  Description of Artificial Sequence Synthetic primer
gccaggagaggagttgggaa

SEQ ID NO: 119 - DNA, 19 bp, Artificial Sequence
  Description of Artificial Sequence Synthetic primer
cgctgtgtcatccaacggg

SEQ ID NO: 120 - DNA, 24 bp, Artificial Sequence
  Description of Artificial Sequence Synthetic primer
tggatatacctggaagagcacctt

SEQ ID NO: 121 - DNA, 24 bp, Artificial Sequence
  Description of Artificial Sequence Synthetic primer
cccttctcccatgttttcttcctc

SEQ ID NO: 122 - DNA, 24 bp, Artificial Sequence
  Description of Artificial Sequence Synthetic primer
cggttaccgtgatcaaaatctcca

SEQ ID NO: 123 - DNA, 19 bp, Artificial Sequence
  Description of Artificial Sequence Synthetic primer
agcccgaattcacccagga

SEQ ID NO: 124 - DNA, 21 bp, Artificial Sequence
  Description of Artificial Sequence Synthetic primer
aacctttgggcttggacaaca

SEQ ID NO: 125 - DNA, 19 bp, Artificial Sequence
  Description of Artificial Sequence Synthetic primer
cccagtcccaaagtgcagc

SEQ ID NO: 126 - DNA, 20 bp, Artificial Sequence
  Description of Artificial Sequence Synthetic primer
ggctgaggatggtgtaagcg

SEQ ID NO: 127 - DNA, 18 bp, Artificial Sequence
  Description of Artificial Sequence Synthetic primer
ccacagacgcggacgatg

SEQ ID NO: 128 - DNA, 18 bp, Artificial Sequence
  Description of Artificial Sequence Synthetic primer
tccagcccagtggtgacc

SEQ ID NO: 129 - DNA, 23 bp, Artificial Sequence
  Description of Artificial Sequence Synthetic primer
acaggaacacaggagtcatcagt

SEQ ID NO: 130 - DNA, 20 bp, Artificial Sequence
  Description of Artificial Sequence Synthetic primer
tgacaactggcctagcagga

SEQ ID NO: 131 - DNA, 22 bp, Artificial Sequence
  Description of Artificial Sequence Synthetic primer
caaaggtggctagtgttcctgg

SEQ ID NO: 132 - DNA, 23 bp, Artificial Sequence
  Description of Artificial Sequence Synthetic primer
tggtgtcagtgactgtgatcaca

SEQ ID NO: 133 - DNA, 21 bp, Artificial Sequence
  Description of Artificial Sequence Synthetic primer
gaggggttaagcacaacagca

SEQ ID NO: 134 - DNA, 26 bp, Artificial Sequence
  Description of Artificial Sequence Synthetic primer
tggtgacacttagttcatgaacaaca

SEQ ID NO: 135 - DNA, 18 bp, Artificial Sequence
  Description of Artificial Sequence Synthetic primer
gccccctgagactcagct

SEQ ID NO: 136 - DNA, 19 bp, Artificial Sequence
  Description of Artificial Sequence Synthetic primer
tgggggcatcagcatcagt

SEQ ID NO: 137 - DNA, 23 bp, Artificial Sequence
  Description of Artificial Sequence Synthetic primer
gctaacgtcgtaatcaccacact

SEQ ID NO: 138 - DNA, 22 bp, Artificial Sequence
  Description of Artificial Sequence Synthetic primer
aatgccatcgttgttcactgga

SEQ ID NO: 139 - DNA, 26 bp, Artificial Sequence
  Description of Artificial Sequence Synthetic primer
accatattgaatgatgatggtggaca

SEQ ID NO: 140 - DNA, 22 bp, Artificial Sequence
  Description of Artificial Sequence Synthetic primer
ccaggggacaagggtatgaaca

SEQ ID NO: 141 - DNA, 18 bp, Artificial Sequence
  Description of Artificial Sequence Synthetic primer
ctgcccaggagccagaca

SEQ ID NO: 142 - DNA, 26 bp, Artificial Sequence
  Description of Artificial Sequence Synthetic primer
acagacaaatgacaaaatgccatgaa

SEQ ID NO: 143 - DNA, 19 bp, Artificial Sequence
  Description of Artificial Sequence Synthetic primer
gcccccatctttgtgcctc

SEQ ID NO: 144 - DNA, 26 bp, Artificial Sequence
  Description of Artificial Sequence Synthetic primer
gcaagtcagttgaaaaatcctcacac

SEQ ID NO: 145 - DNA, 22 bp, Artificial Sequence
  Description of Artificial Sequence Synthetic primer
gcagtgacgaatgtggtacctt

SEQ ID NO: 146 - DNA, 18 bp, Artificial Sequence
  Description of Artificial Sequence Synthetic primer
gcccacgccaaagtcctc

SEQ ID NO: 147 - DNA, 24 bp, Artificial Sequence
  Description of Artificial Sequence Synthetic primer
ttcattgtttctgctctctagggc

SEQ ID NO: 148 - DNA, 20 bp, Artificial Sequence
  Description of Artificial Sequence Synthetic primer
cacatccagcacatccacgg

SEQ ID NO: 149 - DNA, 23 bp, Artificial Sequence
  Description of Artificial Sequence Synthetic primer
gctacatgttgtttgctggtcct

SEQ ID NO: 150 - DNA, 18 bp, Artificial Sequence
  Description of Artificial Sequence Synthetic primer
tccctgtccagctcagcc

SEQ ID NO: 151 - DNA, 18 bp, Artificial Sequence
  Description of Artificial Sequence Synthetic primer
tccggacactggtgccat

SEQ ID NO: 152 - DNA, 18 bp, Artificial Sequence
  Description of Artificial Sequence Synthetic primer
ggtcgaggcagcaaaggc

SEQ ID NO: 153 - DNA, 23 bp, Artificial Sequence
  Description of Artificial Sequence Synthetic primer
tctagacttggtctggtggaagg

SEQ ID NO: 154 - DNA, 25 bp, Artificial Sequence
  Description of Artificial Sequence Synthetic primer
tgtcattcacatcagacaggatcag

SEQ ID NO: 155 - DNA, 20 bp, Artificial Sequence
  Description of Artificial Sequence Synthetic primer
cccccataccagaacctcga

SEQ ID NO: 156 - DNA, 18 bp, Artificial Sequence
  Description of Artificial Sequence Synthetic primer
ggtccagttggcactcgc

SEQ ID NO: 157 - DNA, 23 bp, Artificial Sequence
  Description of Artificial Sequence Synthetic primer
cccaatacatctcccttcacagc

SEQ ID NO: 158 - DNA, 18 bp, Artificial Sequence
  Description of Artificial Sequence Synthetic primer
aggtggggatctggggga

SEQ ID NO: 159 - DNA, 25 bp, Artificial Sequence
  Description of Artificial Sequence Synthetic primer
tgagctttttattttcctcccctgg

SEQ ID NO: 160 - DNA, 26 bp, Artificial Sequence
  Description of Artificial Sequence Synthetic primer
tctggttatccatgagcttgagattg

SEQ ID NO: 161 - DNA, 20 bp, Artificial Sequence
  Description of Artificial Sequence Synthetic primer
tggccttagaggtgggtgac

SEQ ID NO: 162 - DNA, 19 bp, Artificial Sequence
  Description of Artificial Sequence Synthetic primer
tcctgcttcgacaggctgt

SEQ ID NO: 163 - DNA, 20 bp, Artificial Sequence
  Description of Artificial Sequence Synthetic primer
gcgtgtgtgactgtgaaggg

SEQ ID NO: 164 - DNA, 24 bp, Artificial Sequence
  Description of Artificial Sequence Synthetic primer
cagctggacttacttagcaaagca

SEQ ID NO: 165 - DNA, 19 bp, Artificial Sequence
  Description of Artificial Sequence Synthetic primer
gaggatgacacccgggaca

SEQ ID NO: 166 - DNA, 26 bp, Artificial Sequence
  Description of Artificial Sequence Synthetic primer
gctgtttcaaatgcctacctcttact

SEQ ID NO: 167 - DNA, 20 bp, Artificial Sequence
  Description of Artificial Sequence Synthetic primer
ccccaccatcccagttctga

SEQ ID NO: 168 - DNA, 20 bp, Artificial Sequence
  Description of Artificial Sequence Synthetic primer
cctcttctccgcctccttct

SEQ ID NO: 169 - DNA, 20 bp, Artificial Sequence
  Description of Artificial Sequence Synthetic primer
ctaactgccccctgtctggt

SEQ ID NO: 170 - DNA, 21 bp, Artificial Sequence
  Description of Artificial Sequence Synthetic primer
gctctcctccgaagaaacagc

SEQ ID NO: 171 - DNA, 25 bp, Artificial Sequence
  Description of Artificial Sequence Synthetic primer
aactgaacatagccctgtgtgtatg

SEQ ID NO: 172 - DNA, 18 bp, Artificial Sequence
  Description of Artificial Sequence Synthetic primer
gggttggtgcaacgtcgt

SEQ ID NO: 173 - DNA, 18 bp, Artificial Sequence
  Description of Artificial Sequence Synthetic primer
tatcttccccgccctgcc

SEQ ID NO: 174 - DNA, 30 bp, Artificial Sequence
  Description of Artificial Sequence Synthetic primer
agactctttttctcatttttgacacaactc

SEQ ID NO: 175 - DNA, 20 bp, Artificial Sequence
  Description of Artificial Sequence Synthetic primer
gctgctagtctgagctccct

SEQ ID NO: 176 - DNA, 21 bp, Artificial Sequence
  Description of Artificial Sequence Synthetic primer
gcctctctcgagtcccctagt

SEQ ID NO: 177 - DNA, 28 bp, Artificial Sequence
  Description of Artificial Sequence Synthetic primer
tgtcgtaccttacatattgctagacttc

SEQ ID NO: 178 - DNA, 21 bp, Artificial Sequence
  Description of Artificial Sequence Synthetic primer
agagaatcataaggcggggct

SEQ ID NO: 179 - DNA, 23 bp, Artificial Sequence
  Description of Artificial Sequence Synthetic primer
cttcaagaagctggctgacatgt

SEQ ID NO: 180 - DNA, 22 bp, Artificial Sequence
  Description of Artificial Sequence Synthetic primer
actcatctcaagggaagggagc

SEQ ID NO: 181 - DNA, 18 bp, Artificial Sequence
  Description of Artificial Sequence Synthetic primer
gcacctcccgctcctgga

SEQ ID NO: 182 - DNA, 21 bp, Artificial Sequence
  Description of Artificial Sequence Synthetic primer
gttggaggcagtagaagggga

SEQ ID NO: 183 - DNA, 20 bp, Artificial Sequence
  Description of Artificial Sequence Synthetic primer
cctgtcaccatttccagggc

SEQ ID NO: 184 - DNA, 19 bp, Artificial Sequence
  Description of Artificial Sequence Synthetic primer
ggcgcacgggaggtttaaa

SEQ ID NO: 185 - DNA, 18 bp, Artificial Sequence
  Description of Artificial Sequence Synthetic primer
tggcacatccagggaccc

SEQ ID NO: 186 - DNA, 18 bp, Artificial Sequence
  Description of Artificial Sequence Synthetic primer
ggcagcggaatggggaga

SEQ ID NO: 187 - DNA, 21 bp, Artificial Sequence
  Description of Artificial Sequence Synthetic primer
ccagagccatttccatcctgc

SEQ ID NO: 188 - DNA, 28 bp, Artificial Sequence
  Description of Artificial Sequence Synthetic primer
tcctcttgatatctccttttgtttctgc

SEQ ID NO: 189 - DNA, 30 bp, Artificial Sequence
  Description of Artificial Sequence Synthetic primer
atagtattaatgtaatttcaaatgttagct

SEQ ID NO: 190 - DNA, 29 bp, Artificial Sequence
  Description of Artificial Sequence Synthetic primer
gcaagcatacaaataagaaaacatactta

SEQ ID NO: 191 - DNA, 25 bp, Artificial Sequence
  Description of Artificial Sequence Synthetic primer
tgtgcatatttattacatcggggca

SEQ ID NO: 192 - DNA, 28 bp, Artificial Sequence
  Description of Artificial Sequence Synthetic primer
ccaataaattctcagatccaggaagagg

SEQ ID NO: 193 - DNA, 19 bp, Artificial Sequence
  Description of Artificial Sequence Synthetic primer
ctgtccaccagggagtaac

SEQ ID NO: 194 - DNA, 19 bp, Artificial Sequence
  Description of Artificial Sequence Synthetic primer
ttccgccactgaacattgg

SEQ ID NO: 195 - DNA, 27 bp, Artificial Sequence
  Description of Artificial Sequence Synthetic primer
cttccacaaacagaacaagatgctaaa

SEQ ID NO: 196 - DNA, 30 bp, Artificial Sequence
  Description of Artificial Sequence Synthetic primer
aaaacacctgcagatctaatagaaaacaaa

SEQ ID NO: 197 - DNA, 26 bp, Artificial Sequence
  Description of Artificial Sequence Synthetic primer
cgggaagacaagttcatgtactttga

SEQ ID NO: 198 - DNA, 30 bp, Artificial Sequence
  Description of Artificial Sequence Synthetic primer
tgtccttattttggatatttctcccaatga

SEQ ID NO: 199 - DNA, 30 bp, Artificial Sequence
  Description of Artificial Sequence Synthetic primer
acagaatccatatttcgtgtatattgctga

SEQ ID NO: 200 - DNA, 21 bp, Artificial Sequence
  Description of Artificial Sequence Synthetic primer
cacctttagctggcagaccac

SEQ ID NO: 201 - DNA, 30 bp, Artificial Sequence
  Description of Artificial Sequence Synthetic primer
tagatattctgacaccactgactctgatcc

SEQ ID NO: 202 - DNA, 32 bp, Artificial Sequence
  Description of Artificial Sequence Synthetic primer
aaggtccattttcagtttattcaagtttattt

SEQ ID NO: 203 - DNA, 30 bp, Artificial Sequence
  Description of Artificial Sequence Synthetic primer
agatgagtcatatttgtgggttttcatttt

SEQ ID NO: 204 - DNA, 26 bp, Artificial Sequence
  Description of Artificial Sequence Synthetic primer
tcaggttcattgtcactaacatctgg

SEQ ID NO: 205 - DNA, 25 bp, Artificial Sequence
  Description of Artificial Sequence Synthetic primer
gatagcatttgcagtatagagcgtg

SEQ ID NO: 206 - DNA, 26 bp, Artificial Sequence
  Description of Artificial Sequence Synthetic primer
aacccccacaaaatgtttaatttaac

SEQ ID NO: 207 - DNA, 21 bp, Artificial Sequence
  Description of Artificial Sequence Synthetic primer
tggagaagggaagtcggaaca

SEQ ID NO: 208 - DNA, 18 bp, Artificial Sequence
  Description of Artificial Sequence Synthetic primer
caccgacatcagctcgcc

SEQ ID NO: 209 - DNA, 18 bp, Artificial Sequence
  Description of Artificial Sequence Synthetic primer
gcagcagctgggcatgtt

SEQ ID NO: 210 - DNA, 18 bp, Artificial Sequence
  Description of Artificial Sequence Synthetic primer
gcaggtcccccatcaggt

SEQ ID NO: 211 - DNA, 18 bp, Artificial Sequence
  Description of Artificial Sequence Synthetic primer
catctaccagccgcgccg

SEQ ID NO: 212 - DNA, 20 bp, Artificial Sequence
  Description of Artificial Sequence Synthetic primer
tgaggatcttgacggccctc

SEQ ID NO: 213 - DNA, 18 bp, Artificial Sequence
  Description of Artificial Sequence Synthetic primer
aggaggtgctggactcgg

SEQ ID NO: 214 - DNA, 22 bp, Artificial Sequence
  Description of Artificial Sequence Synthetic primer
accccagcaagccatacttact

SEQ ID NO: 215 - DNA, 18 bp, Artificial Sequence
  Description of Artificial Sequence Synthetic primer
cggggaggccaacgtgaa

SEQ ID NO: 216 - DNA, 22 bp, Artificial Sequence
  Description of Artificial Sequence Synthetic primer
ggtccagctcagggtgttaaga

SEQ ID NO: 217 - DNA, 18 bp, Artificial Sequence
  Description of Artificial Sequence Synthetic primer
gggcctgtggtgtttggg

SEQ ID NO: 218 - DNA, 18 bp, Artificial Sequence
  Description of Artificial Sequence Synthetic primer
tgccctggctatgcaggt

SEQ ID NO: 219 - DNA, 21 bp, Artificial Sequence
  Description of Artificial Sequence Synthetic primer
cgcaggtacttctgtcagctg

SEQ ID NO: 220 - DNA, 18 bp, Artificial Sequence
  Description of Artificial Sequence Synthetic primer
gcctacctcggccacgcc

SEQ ID NO: 221 - DNA, 18 bp, Artificial Sequence
  Description of Artificial Sequence Synthetic primer
accggtggcaccctcaaa

SEQ ID NO: 222 - DNA, 18 bp, Artificial Sequence
  Description of Artificial Sequence Synthetic primer
gaacgggtgcagtgcctg

SEQ ID NO: 223 - DNA, 20 bp, Artificial Sequence
  Description of Artificial Sequence Synthetic primer
ctctgtccctggggtagagc

SEQ ID NO: 224 - DNA, 25 bp, Artificial Sequence
  Description of Artificial Sequence Synthetic primer
acaacttgtagatgttgtccccttc

SEQ ID NO: 225 - DNA, 21 bp, Artificial Sequence
  Description of Artificial Sequence Synthetic primer
agctacaacatcaccacgggt

SEQ ID NO: 226 - DNA, 19 bp, Artificial Sequence
  Description of Artificial Sequence Synthetic primer
aggctcccacctttcagca

SEQ ID NO: 227 - DNA, 18 bp, Artificial Sequence
  Description of Artificial Sequence Synthetic primer
catcccgggcgactgtgg

SEQ ID NO: 228 - DNA, 18 bp, Artificial Sequence
  Description of Artificial Sequence Synthetic primer
ggggtctcggggccaata

SEQ ID NO: 229 - DNA, 18 bp, Artificial Sequence
  Description of Artificial Sequence Synthetic primer
tggtgccagcctgacagg

SEQ ID NO: 230 - DNA, 18 bp, Artificial Sequence
  Description of Artificial Sequence Synthetic primer
cagtcatgctgcgccacc

SEQ ID NO: 231 - DNA, 19 bp, Artificial Sequence
  Description of Artificial Sequence Synthetic primer
cgagcccagacaccaagga

SEQ ID NO: 232 - DNA, 18 bp, Artificial Sequence
  Description of Artificial Sequence Synthetic primer
gccagactcaccgggcac

SEQ ID NO: 233 - DNA, 26 bp, Artificial Sequence
  Description of Artificial Sequence Synthetic primer
tgacatcatctacactcaggacttca

SEQ ID NO: 234 - DNA, 19 bp, Artificial Sequence
  Description of Artificial Sequence Synthetic primer
accagagggcagaagctgt

SEQ ID NO: 235 - DNA, 23 bp, Artificial Sequence
  Description of Artificial Sequence Synthetic primer
ctctgtctccttcctcttcctac

SEQ ID NO: 236 - DNA, 20 bp, Artificial Sequence
  Description of Artificial Sequence Synthetic primer
gtgctgtgactgcttgtaga

SEQ ID NO: 237 - DNA, 19 bp, Artificial Sequence
  Description of Artificial Sequence Synthetic primer
ctgtgcagctgtgggttga

SEQ ID NO: 238 - DNA, 19 bp, Artificial Sequence
  Description of Artificial Sequence Synthetic primer
catgacggaggttgtgagg

SEQ ID NO: 239 - DNA, 23 bp, Artificial Sequence
  Description of Artificial Sequence Synthetic primer
caggtaggacctgatttccttac

SEQ ID NO: 240 - DNA, 21 bp, Artificial Sequence
  Description of Artificial Sequence Synthetic primer
ttcttgcggagattctcttcc

SEQ ID NO: 241 - DNA, 20 bp, Artificial Sequence
  Description of Artificial Sequence Synthetic primer
tgggacggaacagctttgag

SEQ ID NO: 242 - DNA, 18 bp, Artificial Sequence
  Description of Artificial Sequence Synthetic primer
ccaccgcttcttgtcctg

SEQ ID NO: 243 - DNA, 19 bp, Artificial Sequence
  Description of Artificial Sequence Synthetic primer
gggtgcagttatgcctcag

SEQ ID NO: 244 - DNA, 22 bp, Artificial Sequence
  Description of Artificial Sequence Synthetic primer
agacttagtacctgaagggtga

SEQ ID NO: 245 - DNA, 20 bp, Artificial Sequence
  Description of Artificial Sequence Synthetic primer
tagcactgcccaacaacacc

SEQ ID NO: 246 - DNA, 23 bp, Artificial Sequence
  Description of Artificial Sequence Synthetic primer
cggcattttgagtgttagactgg

SEQ ID NO: 247 - DNA, 20 bp, Artificial Sequence
  Description of Artificial Sequence Synthetic primer
ttacttctccccctcctctg

SEQ ID NO: 248 - DNA, 18 bp, Artificial Sequence
  Description of Artificial Sequence Synthetic primer
cttcccagcctgggcatc

SEQ ID NO: 249 - DNA, 20 bp, Artificial Sequence
  Description of Artificial Sequence Synthetic primer
gctgaatgaggccttggaac

SEQ ID NO: 250 - DNA, 21 bp, Artificial Sequence
  Description of Artificial Sequence Synthetic primer
ctttccaacctaggaaggcag

SEQ ID NO: 251 - DNA, 19 bp, Artificial Sequence
  Description of Artificial Sequence Synthetic primer
tcctccctgcttctgtctc

SEQ ID NO: 252 - DNA, 21 bp, Artificial Sequence
  Description of Artificial Sequence Synthetic primer
ctgtcagtggggaacaagaag

SEQ ID NO: 253 - DNA, 18 bp, Artificial Sequence
  Description of Artificial Sequence Synthetic primer
tcttgcagcagccagact

SEQ ID NO: 254 - DNA, 19 bp, Artificial Sequence
  Description of Artificial Sequence Synthetic primer
cctgcccttccaatggatc

SEQ ID NO: 255 - DNA, 18 bp, Artificial Sequence
  Description of Artificial Sequence Synthetic primer
cccctagcagagacctgt

SEQ ID NO: 256 - DNA, 19 bp, Artificial Sequence
  Description of Artificial Sequence Synthetic primer
gcccaacccttgtccttac

SEQ ID NO: 257 - DNA, 21 bp, Artificial Sequence
  Description of Artificial Sequence Synthetic primer
ctgactgctcttttcacccat

SEQ ID NO: 258 - DNA, 20 bp, Artificial Sequence
  Description of Artificial Sequence Synthetic primer
gagcagcctctggcattctg

SEQ ID NO: 259 - DNA, 21 bp, Artificial Sequence
  Description of Artificial Sequence Synthetic primer
tgaagacccaggtccagatga

SEQ ID NO: 260 - DNA, 21 bp, Artificial Sequence
  Description of Artificial Sequence Synthetic primer
gctgccctggtaggttttctg

SEQ ID NO: 261 - DNA, 20 bp, Artificial Sequence
  Description of Artificial Sequence Synthetic primer
ctggcccctgtcatcttctg

SEQ ID NO: 262 - DNA, 21 bp, Artificial Sequence
  Description of Artificial Sequence Synthetic primer
caggcattgaagtctcatgga

SEQ ID NO: 263 - DNA, 20 bp, Artificial Sequence
  Description of Artificial Sequence Synthetic primer
gctcaccatcgctatctgag

SEQ ID NO: 264 - DNA, 22 bp, Artificial Sequence
  Description of Artificial Sequence Synthetic primer
agcaatcagtgaggaatcagag

SEQ ID NO: 265 - DNA, 17 bp, Artificial Sequence
  Description of Artificial Sequence Synthetic primer
agctggggctggagaga

SEQ ID NO: 266 - DNA, 23 bp, Artificial Sequence
  Description of Artificial Sequence Synthetic primer
gtcatccaaatactccacacgca

SEQ ID NO: 267 - DNA, 21 bp, Artificial Sequence
  Description of Artificial Sequence Synthetic primer
gcatcttatccgagtggaagg

SEQ ID NO: 268 - DNA, 21 bp, Artificial Sequence
  Description of Artificial Sequence Synthetic primer
cactgacaaccacccttaacc

SEQ ID NO: 269 - DNA, 18 bp, Artificial Sequence
  Description of Artificial Sequence Synthetic primer
cgcactggcctcatcttg

SEQ ID NO: 270 - DNA, 22 bp, Artificial Sequence
  Description of Artificial Sequence Synthetic primer
cttccagtgtgatgatggtgag

SEQ ID NO: 271 - DNA, 22 bp, Artificial Sequence
  Description of Artificial Sequence Synthetic primer
catgtgtaacagttcctgcatg

SEQ ID NO: 272 - DNA, 18 bp, Artificial Sequence
  Description of Artificial Sequence Synthetic primer
ggtcagaggcaagcagag

SEQ ID NO: 273 - DNA, 23 bp, Artificial Sequence
  Description of Artificial Sequence Synthetic primer
cctggttgtagctaactaacttc

SEQ ID NO: 274 - DNA, 23 bp, Artificial Sequence
  Description of Artificial Sequence Synthetic primer
accatcgtaagtcaagtagcatc

SEQ ID NO: 275 - DNA, 22 bp, Artificial Sequence
  Description of Artificial Sequence Synthetic primer
atggttctatgactttgcctga

SEQ ID NO: 276 - DNA, 21 bp, Artificial Sequence
  Description of Artificial Sequence Synthetic primer
agcaggctaggctaagctatg

SEQ ID NO: 277 - DNA, 25 bp, Artificial Sequence
  Description of Artificial Sequence Synthetic primer
tgatttaggtttctgctttgggaca

SEQ ID NO: 278 - DNA, 19 bp, Artificial Sequence
  Description of Artificial Sequence Synthetic primer
tgccccacagttcacctga

SEQ ID NO: 279 - DNA, 21 bp, Artificial Sequence
  Description of Artificial Sequence Synthetic primer
cagaacaatgcctccacgacc

SEQ ID NO: 280 - DNA, 26 bp, Artificial Sequence
  Description of Artificial Sequence Synthetic primer
atggttattaatgtagcctcacggag

SEQ ID NO: 281 - DNA, 30 bp, Artificial Sequence
  Description of Artificial Sequence Synthetic primer
tgtttactaccaaatggaatgatagtgact

SEQ ID NO: 282 - DNA, 22 bp, Artificial Sequence
  Description of Artificial Sequence Synthetic primer
tgaagaagttgatggagggggt

SEQ ID NO: 283 - DNA, 25 bp, Artificial Sequence
  Description of Artificial Sequence Synthetic primer
agtgttactcaagaagcagaaaggg

SEQ ID NO: 284 - DNA, 28 bp, Artificial Sequence
  Description of Artificial Sequence Synthetic primer
tcataccaatttctcgattgaggatctt

SEQ ID NO: 285 - DNA, 25 bp, Artificial Sequence
  Description of Artificial Sequence Synthetic primer
tgcaccattgatgtctacatgatca

SEQ ID NO: 286 - DNA, 19 bp, Artificial Sequence
  Description of Artificial Sequence Synthetic primer
ggtcccctttcatgcccct

SEQ ID NO: 287 - DNA, 18 bp, Artificial Sequence
  Description of Artificial Sequence Synthetic primer
cagcaagcacacagggcc

SEQ ID NO: 288 - DNA, 18 bp, Artificial Sequence
  Description of Artificial Sequence Synthetic primer
cacaaagcgctgggggtc

SEQ ID NO: 289 - DNA, 20 bp, Artificial Sequence
  Description of Artificial Sequence Synthetic primer
gaattctcccgcatggccag

SEQ ID NO: 290 - DNA, 18 bp, Artificial Sequence
  Description of Artificial Sequence Synthetic primer
aggggcctggcatactgg

SEQ ID NO: 291 - DNA, 20 bp, Artificial Sequence
  Description of Artificial Sequence Synthetic primer
tgcctctccttcctccacag

SEQ ID NO: 292 - DNA, 20 bp, Artificial Sequence
  Description of Artificial Sequence Synthetic primer
tggacagaagaagccctgct

SEQ ID NO: 293 - DNA, 20 bp, Artificial Sequence
  Description of Artificial Sequence Synthetic primer
ggacctggtggatgctgagg

SEQ ID NO: 294 - DNA, 19 bp, Artificial Sequence
  Description of Artificial Sequence Synthetic primer
acacagtgtgaccgagggc

SEQ ID NO: 295 - DNA, 18 bp, Artificial Sequence
  Description of Artificial Sequence Synthetic primer
tggtccaccacaggcacc

SEQ ID NO: 296 - DNA, 21 bp, Artificial Sequence
  Description of Artificial Sequence Synthetic primer
tggggtttccttgagaggtga

SEQ ID NO: 297 - DNA, 20 bp, Artificial Sequence
  Description of Artificial Sequence Synthetic primer
tcaccttccatggagtcccc

SEQ ID NO: 298 - DNA, 18 bp, Artificial Sequence
  Description of Artificial Sequence Synthetic primer
cctgggggcctcctcttc

SEQ ID NO: 299 - DNA, 20 bp, Artificial Sequence
  Description of Artificial Sequence Synthetic primer
ggacctgacactagggctgg

SEQ ID NO: 300 - DNA, 19 bp, Artificial Sequence
  Description of Artificial Sequence Synthetic primer
gtgtggggaggctttgcag

SEQ ID NO: 301 - DNA, 18 bp, Artificial Sequence
  Description of Artificial Sequence Synthetic primer
ccagccctctacagcggt

SEQ ID NO: 302 - DNA, 18 bp, Artificial Sequence
  Description of Artificial Sequence Synthetic primer
cacctcccctgcccatca

SEQ ID NO: 303 - DNA, 21 bp, Artificial Sequence
  Description of Artificial Sequence Synthetic primer
cagcctggtatggagtccagt

SEQ ID NO: 304 - DNA, 25 bp, Artificial Sequence
  Description of Artificial Sequence Synthetic primer
ttctgaaaggtcaagagaaggtgac

SEQ ID NO: 305 - DNA, 18 bp, Artificial Sequence
  Description of Artificial Sequence Synthetic primer
agtggcagagacaccggg

SEQ ID NO: 306 - DNA, 18 bp, Artificial Sequence
  Description of Artificial Sequence Synthetic primer
tccagagtggcaccagca

SEQ ID NO: 307 - DNA, 20 bp, Artificial Sequence
  Description of Artificial Sequence Synthetic primer
acgtttttgcctttgggggt

SEQ ID NO: 308 - DNA, 18 bp, Artificial Sequence
  Description of Artificial Sequence Synthetic primer
gctctggtgggtcctggt

SEQ ID NO: 309 - DNA, 18 bp, Artificial Sequence
  Description of Artificial Sequence Synthetic primer
tcctcctgccttcagccc

SEQ ID NO: 310 - DNA, 18 bp, Artificial Sequence
  Description of Artificial Sequence Synthetic primer
tggcacgtccagacccag

SEQ ID NO: 311 - DNA, 20 bp, Artificial Sequence
  Description of Artificial Sequence Synthetic primer
cctacggcagagaacccaga

SEQ ID NO: 312 - DNA, 18 bp, Artificial Sequence
  Description of Artificial Sequence Synthetic primer
gaagtggtcggagggccc

SEQ ID NO: 313 - DNA, 19 bp, Artificial Sequence
  Description of Artificial Sequence Synthetic primer
gagcagggaaggcctgact

SEQ ID NO: 314 - DNA, 18 bp, Artificial Sequence
  Description of Artificial Sequence Synthetic primer
acgaggctggaccccttc

SEQ ID NO: 315 - DNA, 21 bp, Artificial Sequence
  Description of Artificial Sequence Synthetic primer
tccttcctgcttgagttccca

SEQ ID NO: 316 - DNA, 18 bp, Artificial Sequence
  Description of Artificial Sequence Synthetic primer
ggaaagggccaagctggg

SEQ ID NO: 317 - DNA, 18 bp, Artificial Sequence
  Description of Artificial Sequence Synthetic primer
aagctggcctgagagggg

SEQ ID NO: 318 - DNA, 22 bp, Artificial Sequence
  Description of Artificial Sequence Synthetic primer
aagcactctgtacaaagcctgg

SEQ ID NO: 319 - DNA, 22 bp, Artificial Sequence
  Description of Artificial Sequence Synthetic primer
ggaaggaacagcaatggtgtca

SEQ ID NO: 320 - DNA, 18 bp, Artificial Sequence
  Description of Artificial Sequence Synthetic primer
cccacacttgcctcccca

SEQ ID NO: 321 - DNA, 19 bp, Artificial Sequence
  Description of Artificial Sequence Synthetic primer
ccagggggagaatgggtgt

SEQ ID NO: 322 - DNA, 18 bp, Artificial Sequence
  Description of Artificial Sequence Synthetic primer
acagagccacccccagac

SEQ ID NO: 323 - DNA, 28 bp, Artificial Sequence
  Description of Artificial Sequence Synthetic primer
aatttgtagaccctcttaagatcatgct

SEQ ID NO: 324 - DNA, 22 bp, Artificial Sequence
  Description of Artificial Sequence Synthetic primer
tggttttcccaccacatcctct

SEQ ID NO: 325 - DNA, 18 bp, Artificial Sequence
  Description of Artificial Sequence Synthetic primer
ccgcagtgagcaccatgg

SEQ ID NO: 326 - DNA, 18 bp, Artificial Sequence
  Description of Artificial Sequence Synthetic primer
atccacagggcagggtcc

SEQ ID NO: 327 - DNA, 18 bp, Artificial Sequence
  Description of Artificial Sequence Synthetic primer
gatggggtggccaggtct

SEQ ID NO: 328 - DNA, 18 bp, Artificial Sequence
  Description of Artificial Sequence Synthetic primer
ccctggtagaggtggcgg

SEQ ID NO: 329 - DNA, 18 bp, Artificial Sequence
  Description of Artificial Sequence Synthetic primer
cccgagacccacctggac

SEQ ID NO: 330 - DNA, 18 bp, Artificial Sequence
  Description of Artificial Sequence Synthetic primer
cagggcctggctgggttg

SEQ ID NO: 331 - DNA, 20 bp, Artificial Sequence
  Description of Artificial Sequence Synthetic primer
caaccaagtgaggcaggtcc

SEQ ID NO: 332 - DNA, 22 bp, Artificial Sequence
  Description of Artificial Sequence Synthetic primer
gtggtattgttcagcgggtctc

SEQ ID NO: 333 - DNA, 18 bp, Artificial Sequence
  Description of Artificial Sequence Synthetic primer
tatgccctggccgtgcta

SEQ ID NO: 334 - DNA, 24 bp, Artificial Sequence
  Description of Artificial Sequence Synthetic primer
accaagagaaggtttcaatgacgg

SEQ ID NO: 335 - DNA, 20 bp, Artificial Sequence
  Description of Artificial Sequence Synthetic primer
tggggaatctgggggttgtt

SEQ ID NO: 336 - DNA, 19 bp, Artificial Sequence
  Description of Artificial Sequence Synthetic primer
ccgctggatcaagacccct

SEQ ID NO: 337 - DNA, 23 bp, Artificial Sequence
  Description of Artificial Sequence Synthetic primer
aagggtcctctgatcattgctca

SEQ ID NO: 338 - DNA, 19 bp, Artificial Sequence
  Description of Artificial Sequence Synthetic primer
agtgtgagagccagctggt

SEQ ID NO: 339 - DNA, 23 bp, Artificial Sequence
  Description of Artificial Sequence Synthetic primer
aggacacgattttgtggaaggac

SEQ ID NO: 340 - DNA, 18 bp, Artificial Sequence
  Description of Artificial Sequence Synthetic primer
gtttgcggctggggtcag

SEQ ID NO: 341 - DNA, 19 bp, Artificial Sequence
  Description of Artificial Sequence Synthetic primer
aacccgtcctctcgctgtt

SEQ ID NO: 342 - DNA, 21 bp, Artificial Sequence
  Description of Artificial Sequence Synthetic primer
cctcagaactctctccccagc

SEQ ID NO: 343 - DNA, 20 bp, Artificial Sequence
  Description of Artificial Sequence Synthetic primer
tccgatgtgtaagggctccc

SEQ ID NO: 344 - DNA, 19 bp, Artificial Sequence
  Description of Artificial Sequence Synthetic primer
cccctcccatgtcacctgt

SEQ ID NO: 345 - DNA, 19 bp, Artificial Sequence
  Description of Artificial Sequence Synthetic primer
cctgggccaggtagtctcc

SEQ ID NO: 346 - DNA, 18 bp, Artificial Sequence
  Description of Artificial Sequence Synthetic primer
acagccaccggcacagac

SEQ ID NO: 347 - DNA, 18 bp, Artificial Sequence
  Description of Artificial Sequence Synthetic primer
gggccccaagcactctga

SEQ ID NO: 348 - DNA, 19 bp, Artificial Sequence
  Description of Artificial Sequence Synthetic primer
tggctggctttcactgtgc

SEQ ID NO: 349 - DNA, 19 bp, Artificial Sequence
  Description of Artificial Sequence Synthetic primer
actgccctattgcccctgg

SEQ ID NO: 350 - DNA, 22 bp, Artificial Sequence
  Description of Artificial Sequence Synthetic primer
cgtgtctgtgttgtaggtgacc

SEQ ID NO: 351 - DNA, 20 bp, Artificial Sequence
  Description of Artificial Sequence Synthetic primer
cagtggcatctgtgagctgc

SEQ ID NO: 352 - DNA, 21 bp, Artificial Sequence
  Description of Artificial Sequence Synthetic primer
ctgtgtttctccctggcactc

SEQ ID NO: 353 - DNA, 18 bp, Artificial Sequence
  Description of Artificial Sequence Synthetic primer
gccccctgcacaaccaag

SEQ ID NO: 354 - DNA, 18 bp, Artificial Sequence
  Description of Artificial Sequence Synthetic primer
gctgggccaggctgcatg

SEQ ID NO: 355 - DNA, 19 bp, Artificial Sequence
  Description of Artificial Sequence Synthetic primer
gcacttgcgagaggtgagg

SEQ ID NO: 356 - DNA, 20 bp, Artificial Sequence
  Description of Artificial Sequence Synthetic primer
tgtctgccctgacactgtct

SEQ ID NO: 357 - DNA, 19 bp, Artificial Sequence
  Description of Artificial Sequence Synthetic primer
tgtccaccctgttcctggc

SEQ ID NO: 358 - DNA, 19 bp, Artificial Sequence
  Description of Artificial Sequence Synthetic primer
tgcagagacagagcccacc

SEQ ID NO: 359 - DNA, 21 bp, Artificial Sequence
  Description of Artificial Sequence Synthetic primer
ccagagcagctccaagtgttt

SEQ ID NO: 360 - DNA, 25 bp, Artificial Sequence
  Description of Artificial Sequence Synthetic primer
tgctgagatgtataggtaacctgca

SEQ ID NO: 361 - DNA, 20 bp, Artificial Sequence
  Description of Artificial Sequence Synthetic primer
ggagtccttgtcctgtcccc

SEQ ID NO: 362 - DNA, 20 bp, Artificial Sequence
  Description of Artificial Sequence Synthetic primer
ttgtgcagaattcgtccccg

SEQ ID NO: 363 - DNA, 19 bp, Artificial Sequence
  Description of Artificial Sequence Synthetic primer
tgcctgacctcagcgtctt

SEQ ID NO: 364 - DNA, 19 bp, Artificial Sequence
  Description of Artificial Sequence Synthetic primer
ggagtactccctcaggccc

SEQ ID NO: 365 - DNA, 20 bp, Artificial Sequence
  Description of Artificial Sequence Synthetic primer
cctttctcccatagtggcgc

SEQ ID NO: 366 - DNA, 21 bp, Artificial Sequence
  Description of Artificial Sequence Synthetic primer
gtgttatggtggatgagggcc

SEQ ID NO: 367 - DNA, 19 bp, Artificial Sequence
  Description of Artificial Sequence Synthetic primer
gagggaactgggcagtgga

SEQ ID NO: 368 - DNA, 19 bp, Artificial Sequence
  Description of Artificial Sequence Synthetic primer
accacactcgtcctctggc

SEQ ID NO: 369 - DNA, 20 bp, Artificial Sequence
  Description of Artificial Sequence Synthetic primer
caccaagctctgctccacac

SEQ ID NO: 370 - DNA, 21 bp, Artificial Sequence
  Description of Artificial Sequence Synthetic primer
tctgcacaagtccaagaacgc

SEQ ID NO: 371 - DNA, 18 bp, Artificial Sequence
  Description of Artificial Sequence Synthetic primer
ccacagccatgcccacag

SEQ ID NO: 372 - DNA, 18 bp, Artificial Sequence
  Description of Artificial Sequence Synthetic primer
cacagctggtggcaggcc

SEQ ID NO: 373 - DNA, 18 bp, Artificial Sequence
  Description of Artificial Sequence Synthetic primer
cccgagggcactgctggg

SEQ ID NO: 374 - DNA, 18 bp, Artificial Sequence
  Description of Artificial Sequence Synthetic primer
catgcacccctccagcca

SEQ ID NO: 375 - DNA, 19 bp, Artificial Sequence
  Description of Artificial Sequence Synthetic primer
gccgagtactgcaggggta

SEQ ID NO: 376 - DNA, 18 bp, Artificial Sequence
  Description of Artificial Sequence Synthetic primer
ggtggcacggcaaacagt

SEQ ID NO: 377 - DNA, 18 bp, Artificial Sequence
  Description of Artificial Sequence Synthetic primer
tctcaggctccccaggga

SEQ ID NO: 378 - DNA, 18 bp, Artificial Sequence
  Description of Artificial Sequence Synthetic primer
caatccccctcgctgccc

SEQ ID NO: 379 - DNA, 20 bp, Artificial Sequence
  Description of Artificial Sequence Synthetic primer
ccttgggaagcacaaagggg

SEQ ID NO: 380 - DNA, 18 bp, Artificial Sequence
  Description of Artificial Sequence Synthetic primer
acgcagaagggagggtcc

SEQ ID NO: 381 - DNA, 18 bp, Artificial Sequence
  Description of Artificial Sequence Synthetic primer
ccagtgtgtggcctgtgc

SEQ ID NO: 382 - DNA, 18 bp, Artificial Sequence
  Description of Artificial Sequence Synthetic primer
gggtgcagttgatggggc

SEQ ID NO: 383 - DNA, 18 bp, Artificial Sequence
  Description of Artificial Sequence Synthetic primer
gatgaggagggcgcatgc

SEQ ID NO: 384 - DNA, 25 bp, Artificial Sequence
  Description of Artificial Sequence Synthetic primer
gatatgacaaagggagagttggtcc

SEQ ID NO: 385 - DNA, 23 bp, Artificial Sequence
  Description of Artificial Sequence Synthetic primer
ttctgcctttgtcaaatggggat

SEQ ID NO: 386 - DNA, 20 bp, Artificial Sequence
  Description of Artificial Sequence Synthetic primer
agcccttgtcatccaggtcc

SEQ ID NO: 387 - DNA, 22 bp, Artificial Sequence
  Description of Artificial Sequence Synthetic primer
gagactgtttctcctgcagctg

SEQ ID NO: 388 - DNA, 19 bp, Artificial Sequence
  Description of Artificial Sequence Synthetic primer
acagcagtgaccacccagc

SEQ ID NO: 389 - DNA, 18 bp, Artificial Sequence
  Description of Artificial Sequence Synthetic primer
cccagccctctgacgtcc

SEQ ID NO: 390 - DNA, 19 bp, Artificial Sequence
  Description of Artificial Sequence Synthetic primer
cacctccgtttcctgcagc

SEQ ID NO: 391 - DNA, 19 bp, Artificial Sequence
  Description of Artificial Sequence Synthetic primer
ccggaagtacacgatgcgg

SEQ ID NO: 392 - DNA, 18 bp, Artificial Sequence
  Description of Artificial Sequence Synthetic primer
aggtgtcagcggctccac

SEQ ID NO: 393 - DNA, 18 bp, Artificial Sequence
  Description of Artificial Sequence Synthetic primer
accaccccctcaccccag

SEQ ID NO: 394 - DNA, 21 bp, Artificial Sequence
  Description of Artificial Sequence Synthetic primer
ggccctgaccttgtagactgt

SEQ ID NO: 395 - DNA, 18 bp, Artificial Sequence
  Description of Artificial Sequence Synthetic primer
ggtgcttggatctggcgc

SEQ ID NO: 396 - DNA, 19 bp, Artificial Sequence
  Description of Artificial Sequence Synthetic primer
cccaaacactgcctccagc

SEQ ID NO: 397 - DNA, 19 bp, Artificial Sequence
  Description of Artificial Sequence Synthetic primer
aggtaggatccagcccacg

SEQ ID NO: 398 - DNA, 21 bp, Artificial Sequence
  Description of Artificial Sequence Synthetic primer
tttgttggctttgggggatgt

SEQ ID NO: 399 - DNA, 22 bp, Artificial Sequence
  Description of Artificial Sequence Synthetic primer
tccagtggccatcaaagtgttg

SEQ ID NO: 400 - DNA, 21 bp, Artificial Sequence
  Description of Artificial Sequence Synthetic primer
tgaagagagaccagagcccag

SEQ ID NO: 401 - DNA, 18 bp, Artificial Sequence
  Description of Artificial Sequence Synthetic primer
tgggggtgtgtggtctcc

SEQ ID NO: 402 - DNA, 19 bp, Artificial Sequence
  Description of Artificial Sequence Synthetic primer
aagctgtgtcaccagctgc

SEQ ID NO: 403 - DNA, 18 bp, Artificial Sequence
  Description of Artificial Sequence Synthetic primer
tgtctcccgccttctggg

SEQ ID NO: 404 - DNA, 18 bp, Artificial Sequence
  Description of Artificial Sequence Synthetic primer
agcaggtcctgggagccc

SEQ ID NO: 405 - DNA, 18 bp, Artificial Sequence
  Description of Artificial Sequence Synthetic primer
gcaggtctctccggagca

SEQ ID NO: 406 - DNA, 18 bp, Artificial Sequence
  Description of Artificial Sequence Synthetic primer
agccgcacatcctccagg

SEQ ID NO: 407 - DNA, 22 bp, Artificial Sequence
  Description of Artificial Sequence Synthetic primer
ccagaaggtctacatgggtgct

SEQ ID NO: 408 - DNA, 18 bp, Artificial Sequence
  Description of Artificial Sequence Synthetic primer
agccagcccgaagtctgt

SEQ ID NO: 409 - DNA, 20 bp, Artificial Sequence
  Description of Artificial Sequence Synthetic primer
cgtgctggtcaagagtccca

SEQ ID NO: 410 - DNA, 18 bp, Artificial Sequence
  Description of Artificial Sequence Synthetic primer
caccactccacccagcct

SEQ ID NO: 411 - DNA, 18 bp, Artificial Sequence
  Description of Artificial Sequence Synthetic primer
ggccacctccccacaaca

SEQ ID NO: 412 - DNA, 22 bp, Artificial Sequence
  Description of Artificial Sequence Synthetic primer
ccccatcacacaccataactcc

SEQ ID NO: 413 - DNA, 18 bp, Artificial Sequence
  Description of Artificial Sequence Synthetic primer
gtccattctccgccggcg

SEQ ID NO: 414 - DNA, 18 bp, Artificial Sequence
  Description of Artificial Sequence Synthetic primer
cacatgctgaggtggccc

SEQ ID NO: 415 - DNA, 18 bp, Artificial Sequence
  Description of Artificial Sequence Synthetic primer
aagctccctctggccctc

SEQ ID NO: 416 - DNA, 18 bp, Artificial Sequence
  Description of Artificial Sequence Synthetic primer
cagccgctcccccttttc

SEQ ID NO: 417 - DNA, 20 bp, Artificial Sequence
  Description of Artificial Sequence Synthetic primer
gctgatgacttttggggcca

SEQ ID NO: 418 - DNA, 18 bp, Artificial Sequence
  Description of Artificial Sequence Synthetic primer
cacagctcagccacgcac

SEQ ID NO: 419 - DNA, 20 bp, Artificial Sequence
  Description of Artificial Sequence Synthetic primer
aggctgttggaagctgcttg

SEQ ID NO: 420 - DNA, 24 bp, Artificial Sequence
  Description of Artificial Sequence Synthetic primer
ggtcagcattatgaaggtccactg

SEQ ID NO: 421 - DNA, 28 bp, Artificial Sequence
  Description of Artificial Sequence Synthetic primer
tttttaatgatgctttctggctggattt

SEQ ID NO: 422 - DNA, 30 bp, Artificial Sequence
  Description of Artificial Sequence Synthetic primer
aattccattaccttttctcttgatcatcca

SEQ ID NO: 423 - DNA, 30 bp, Artificial Sequence
  Description of Artificial Sequence Synthetic primer
actctatgcagaaatctatgcagataagaa

SEQ ID NO: 424 - DNA, 24 bp, Artificial Sequence
  Description of Artificial Sequence Synthetic primer
atggggaacaggaggcaaaataaa

SEQ ID NO: 425 - DNA, 23 bp, Artificial Sequence
  Description of Artificial Sequence Synthetic primer
tgacctgagacaagatgctgtca

SEQ ID NO: 426 - DNA, 30 bp, Artificial Sequence
  Description of Artificial Sequence Synthetic primer
tgtttttggtgaactaacagaagtacaaat

SEQ ID NO: 427 - DNA, 24 bp, Artificial Sequence
  Description of Artificial Sequence Synthetic primer
ggctcagcatactacacatgagag

SEQ ID NO: 428 - DNA, 27 bp, Artificial Sequence
  Description of Artificial Sequence Synthetic primer
ggttaacagagtttcctgagagtttct

SEQ ID NO: 429 - DNA, 24 bp, Artificial Sequence
  Description of Artificial Sequence Synthetic primer
gtgtttgactctagatgctgtgag

SEQ ID NO: 430 - DNA, 23 bp, Artificial Sequence
  Description of Artificial Sequence Synthetic primer
cctgatgagatacacagtctacc

SEQ ID NO: 431 - DNA, 27 bp, Artificial Sequence
  Description of Artificial Sequence Synthetic primer
catttggataaagacactgacttgtgc

SEQ ID NO: 432 - DNA, 30 bp, Artificial Sequence
  Description of Artificial Sequence Synthetic primer
agcactctttagataaacaggtcataaaca

SEQ ID NO: 433 - DNA, 21 bp, Artificial Sequence
  Description of Artificial Sequence Synthetic primer
ctcttcctcggcttctcctga

SEQ ID NO: 434 - DNA, 18 bp, Artificial Sequence
  Description of Artificial Sequence Synthetic primer
cctggagctgcagccgcc

SEQ ID NO: 435 - DNA, 30 bp, Artificial Sequence
  Description of Artificial Sequence Synthetic primer
tcttcctaagtgcaaaagataactttatat

SEQ ID NO: 436 - DNA, 29 bp, Artificial Sequence
  Description of Artificial Sequence Synthetic primer
tagtacagtacattcatacctacctctgc

SEQ ID NO: 437 - DNA, 22 bp, Artificial Sequence
  Description of Artificial Sequence Synthetic primer
agaccagtggcactgttgtttc

SEQ ID NO: 438 - DNA, 29 bp, Artificial Sequence
  Description of Artificial Sequence Synthetic primer
atggttaagaaaactgttccaatacatgg

SEQ ID NO: 439 - DNA, 28 bp, Artificial Sequence
  Description of Artificial Sequence Synthetic primer
tggtatgtatttaaccatgcagatcctc

SEQ ID NO: 440 - DNA, 20 bp, Artificial Sequence
  Description of Artificial Sequence Synthetic primer
ccacacacaggtaacggctg

SEQ ID NO: 441 - DNA, 20 bp, Artificial Sequence
  Description of Artificial Sequence Synthetic primer
ccctgatgctcatgtggctg

SEQ ID NO: 442 - DNA, 22 bp, Artificial Sequence
  Description of Artificial Sequence Synthetic primer
actcctggatattggcactggt

SEQ ID NO: 443 - DNA, 19 bp, Artificial Sequence
  Description of Artificial Sequence Synthetic primer
gccggagagctttgatggg

SEQ ID NO: 444 - DNA, 24 bp, Artificial Sequence
  Description of Artificial Sequence Synthetic primer
gctttctttgcattcttgatcccc

SEQ ID NO: 445 - DNA, 18 bp, Artificial Sequence
  Description of Artificial Sequence Synthetic primer
ccgtgggccccctttgtc

SEQ ID NO: 446 - DNA, 19 bp, Artificial Sequence
  Description of Artificial Sequence Synthetic primer
cccaagaccacgaccagca

SEQ ID NO: 447 - DNA, 19 bp, Artificial Sequence
  Description of Artificial Sequence Synthetic primer
tggtgacctgggaatgggg

SEQ ID NO: 448 - DNA, 21 bp, Artificial Sequence
  Description of Artificial Sequence Synthetic primer
catcagtctcagagggcaggg

SEQ ID NO: 449 - DNA, 19 bp, Artificial Sequence
  Description of Artificial Sequence Synthetic primer
ggccctgcccaatgagact

SEQ ID NO: 450 - DNA, 24 bp, Artificial Sequence
  Description of Artificial Sequence Synthetic primer
cgcttttgttcttagacactccct

SEQ ID NO: 451 - DNA, 26 bp, Artificial Sequence
  Description of Artificial Sequence Synthetic primer
tggacttggtgatagacatgtacaga

SEQ ID NO: 452 - DNA, 24 bp, Artificial Sequence
  Description of Artificial Sequence Synthetic primer
tggtaggcaaacaacattccatga

SEQ ID NO: 453 - DNA, 24 bp, Artificial Sequence
  Description of Artificial Sequence Synthetic primer
ttcccaaggcctttaaactgttca

SEQ ID NO: 454 - DNA, 21 bp, Artificial Sequence
  Description of Artificial Sequence Synthetic primer
acagtgccttcttccactcct

SEQ ID NO: 455 - DNA, 23 bp, Artificial Sequence
  Description of Artificial Sequence Synthetic primer
ggacagcctatttttccctcgac

SEQ ID NO: 456 - DNA, 20 bp, Artificial Sequence
  Description of Artificial Sequence Synthetic primer
ctgtaggtggagtcccaggc

SEQ ID NO: 457 - DNA, 20 bp, Artificial Sequence
  Description of Artificial Sequence Synthetic primer
acaccggggtaacatccacc

SEQ ID NO: 458 - DNA, 20 bp, Artificial Sequence
  Description of Artificial Sequence Synthetic primer
caaccccaaactgtcccacg

SEQ ID NO: 459 - DNA, 19 bp, Artificial Sequence
  Description of Artificial Sequence Synthetic primer
agcggctgatactgacccc

SEQ ID NO: 460 - DNA, 27 bp, Artificial Sequence
  Description of Artificial Sequence Synthetic primer
tcaagtagtcatagtcctggtctttgt

SEQ ID NO: 461 - DNA, 23 bp, Artificial Sequence
  Description of Artificial Sequence Synthetic primer
tgtcagttcaaatccctgttgca

SEQ ID NO: 462 - DNA, 22 bp, Artificial Sequence
  Description of Artificial Sequence Synthetic primer
agccaggcacattctagaaggt

SEQ ID NO: 463 - DNA, 30 bp, Artificial Sequence
  Description of Artificial Sequence Synthetic primer
acctgttaagtttgtatgcaacatttctaa

SEQ ID NO: 464 - DNA, 21 bp, Artificial Sequence
  Description of Artificial Sequence Synthetic primer
agctgtggtgggttatggtct

SEQ ID NO: 465 - DNA, 18 bp, Artificial Sequence
  Description of Artificial Sequence Synthetic primer
cccaccaatgccagcctg

SEQ ID NO: 466 - DNA, 23 bp, Artificial Sequence
  Description of Artificial Sequence Synthetic primer
aacaagagaggaaacagaagggc

SEQ ID NO: 467 - DNA, 18 bp, Artificial Sequence
  Description of Artificial Sequence Synthetic primer
ctgcttccccctcccagg

SEQ ID NO: 468 - DNA, 19 bp, Artificial Sequence
  Description of Artificial Sequence Synthetic primer
caaagagctgggtgcctcg

SEQ ID NO: 469 - DNA, 18 bp, Artificial Sequence
  Description of Artificial Sequence Synthetic primer
ttgcccaacagtgacgcg

SEQ ID NO: 470 - DNA, 19 bp, Artificial Sequence
  Description of Artificial Sequence Synthetic primer
gcagaggcacataccaggc

SEQ ID NO: 471 - DNA, 19 bp, Artificial Sequence
  Description of Artificial Sequence Synthetic primer
tgtgactgcctgtccctgt

SEQ ID NO: 472 - DNA, 18 bp, Artificial Sequence
  Description of Artificial Sequence Synthetic primer
aaggcagctcggcaggaa

SEQ ID NO: 473 - DNA, 19 bp, Artificial Sequence
  Description of Artificial Sequence Synthetic primer
catggtggtgcacgaaggg

SEQ ID NO: 474 - DNA, 20 bp, Artificial Sequence
  Description of Artificial Sequence Synthetic primer
accgctgtgttccatcctct

SEQ ID NO: 475 - DNA, 18 bp, Artificial Sequence
  Description of Artificial Sequence Synthetic primer
ctgcggtcccttcctcct

SEQ ID NO: 476 - DNA, 20 bp, Artificial Sequence
  Description of Artificial Sequence Synthetic primer
ggaactggctgcagttgaca

SEQ ID NO: 477 - DNA, 21 bp, Artificial Sequence
  Description of Artificial Sequence Synthetic primer
tggctgcctcttagaccatgt

SEQ ID NO: 478 - DNA, 20 bp, Artificial Sequence
  Description of Artificial Sequence Synthetic primer
agccccttgtggacataggg

SEQ ID NO: 479 - DNA, 19 bp, Artificial Sequence
  Description of Artificial Sequence Synthetic primer
agagcaccctcctgcagag

SEQ ID NO: 480 - DNA, 18 bp, Artificial Sequence
  Description of Artificial Sequence Synthetic primer
tccaagggactggctggg

SEQ ID NO: 481 - DNA, 19 bp, Artificial Sequence
  Description of Artificial Sequence Synthetic primer
aaaacagctaggcaccggc

SEQ ID NO: 482 - DNA, 21 bp, Artificial Sequence
  Description of Artificial Sequence Synthetic primer
aactggatgtctggctcctca

SEQ ID NO: 483 - DNA, 26 bp, Artificial Sequence
  Description of Artificial Sequence Synthetic primer
tgctatgggatttcctgcagaaagac

SEQ ID NO: 484 - DNA, 31 bp, Artificial Sequence
  Description of Artificial Sequence Synthetic primer
cacaacatgaatataaacatcaatatttgaa

SEQ ID NO: 485 - DNA, 30 bp, Artificial Sequence
  Description of Artificial Sequence Synthetic primer
tggattcaaagcataaaaaccattacaaga

SEQ ID NO: 486 - DNA, 25 bp, Artificial Sequence
  Description of Artificial Sequence Synthetic primer
actctacctcactctaacaagcaga

SEQ ID NO: 487 - DNA, 29 bp, Artificial Sequence
  Description of Artificial Sequence Synthetic primer
tttagttgtgctgaaagacattatgacac

SEQ ID NO: 488 - DNA, 26 bp, Artificial Sequence
  Description of Artificial Sequence Synthetic primer
tctcactcgataatctggatgactca

SEQ ID NO: 489 - DNA, 25 bp, Artificial Sequence
  Description of Artificial Sequence Synthetic primer
tctcttaggttctccagttgctact

SEQ ID NO: 490 - DNA, 25 bp, Artificial Sequence
  Description of Artificial Sequence Synthetic primer
tgatgtttatgacctgaggctttgg

SEQ ID NO: 491 - DNA, 18 bp, Artificial Sequence
  Description of Artificial Sequence Synthetic primer
ggcagccgttcggaggat

SEQ ID NO: 492 - DNA, 18 bp, Artificial Sequence
  Description of Artificial Sequence Synthetic primer
ttggctctggaccgcagc

SEQ ID NO: 493 - DNA, 18 bp, Artificial Sequence
  Description of Artificial Sequence Synthetic primer
caaccatccagcagccgc

SEQ ID NO: 494 - DNA, 18 bp, Artificial Sequence
  Description of Artificial Sequence Synthetic primer
cagaagctgctggtggcg

SEQ ID NO: 495 - DNA, 22 bp, Artificial Sequence
  Description of Artificial Sequence Synthetic primer
caaattcctgccattctgggga

SEQ ID NO: 496 - DNA, 26 bp, Artificial Sequence
  Description of Artificial Sequence Synthetic primer
catttccaggaaataaacctcctcca

SEQ ID NO: 497 - DNA, 18 bp, Artificial Sequence
  Description of Artificial Sequence Synthetic primer
ccagctgcacaggggcct

SEQ ID NO: 498 - DNA, 27 bp, Artificial Sequence
  Description of Artificial Sequence Synthetic primer
ttccacgtggattacttacttcatcaa

SEQ ID NO: 499 - DNA, 21 bp, Artificial Sequence
  Description of Artificial Sequence Synthetic primer
tccagcaccctgaagtctctg

SEQ ID NO: 500 - DNA, 18 bp, Artificial Sequence
  Description of Artificial Sequence Synthetic primer
gaggaggagctgggccag

SEQ ID NO: 501 - DNA, 24 bp, Artificial Sequence
  Description of Artificial Sequence Synthetic primer
gacagccatcatcaaagagatcgt

SEQ ID NO: 502 - DNA, 20 bp, Artificial Sequence
  Description of Artificial Sequence Synthetic primer
tcgcatccgtctactcccac

SEQ ID NO: 503 - DNA, 27 bp, Artificial Sequence
  Description of Artificial Sequence Synthetic primer
gaggttatctttttaccacagttgcac

SEQ ID NO: 504 - DNA, 23 bp, Artificial Sequence
  Description of Artificial Sequence Synthetic primer
ccagctttacagtgaattgctgc

SEQ ID NO: 505 - DNA, 26 bp, Artificial Sequence
  Description of Artificial Sequence Synthetic primer
agatcttgaccaatggctaagtgaag

SEQ ID NO: 506 - DNA, 21 bp, Artificial Sequence
  Description of Artificial Sequence Synthetic primer
tctagggcctcttgtgccttt

SEQ ID NO: 507 - DNA, 22 bp, Artificial Sequence
  Description of Artificial Sequence Synthetic primer
tccagaggctagcagttcaact

SEQ ID NO: 508 - DNA, 30 bp, Artificial Sequence
  Description of Artificial Sequence Synthetic primer
cagacttttgtaatttgtgtatgctgatct

SEQ ID NO: 509 - DNA, 18 bp, Artificial Sequence
  Description of Artificial Sequence Synthetic primer
cacccctcgcagcacccc

SEQ ID NO: 510 - DNA, 18 bp, Artificial Sequence
  Description of Artificial Sequence Synthetic primer
aagagggcgaggaggagc

SEQ ID NO: 511 - DNA, 18 bp, Artificial Sequence
  Description of Artificial Sequence Synthetic primer
agcgggaacaggactgct

SEQ ID NO: 512 - DNA, 18 bp, Artificial Sequence
  Description of Artificial Sequence Synthetic primer
gccctgcacctcctggat

SEQ ID NO: 513 - DNA, 18 bp, Artificial Sequence
  Description of Artificial Sequence Synthetic primer
tgcagatgggggcaaggt

SEQ ID NO: 514 - DNA, 18 bp, Artificial Sequence
  Description of Artificial Sequence Synthetic primer
gcacctgggagggcagaa

SEQ ID NO: 515 - DNA, 21 bp, Artificial Sequence
  Description of Artificial Sequence Synthetic primer
accagtaggcaaccgtgaaga

SEQ ID NO: 516 - DNA, 30 bp, Artificial Sequence
  Description of Artificial Sequence Synthetic primer
agattacgaaggtattggtttagacagaaa

SEQ ID NO: 517 - DNA, 28 bp, Artificial Sequence
  Description of Artificial Sequence Synthetic primer
aaaatgaaaaaccttacaggaaatggct

SEQ ID NO: 518 - DNA, 23 bp, Artificial Sequence
  Description of Artificial Sequence Synthetic primer
aacagtccattggcagttgagaa

SEQ ID NO: 519 - DNA, 23 bp, Artificial Sequence
  Description of Artificial Sequence Synthetic primer
atgcccaatttgatgttgatggc

SEQ ID NO: 520 - DNA, 28 bp, Artificial Sequence
  Description of Artificial Sequence Synthetic primer
ccaaagggattttgtagatgtttctcca

SEQ ID NO: 521 - DNA, 21 bp, Artificial Sequence
  Description of Artificial Sequence Synthetic primer
cgcatttccacagctacacca

SEQ ID NO: 522 - DNA, 30 bp, Artificial Sequence
  Description of Artificial Sequence Synthetic primer
gcatttgactttaccttatcaatgtctcga

SEQ ID NO: 523 - DNA, 28 bp, Artificial Sequence
  Description of Artificial Sequence Synthetic primer
gctatatctgaacaaaaattccgtggtt

SEQ ID NO: 524 - DNA, 24 bp, Artificial Sequence
  Description of Artificial Sequence Synthetic primer
agggttctcctccatggtagatac

SEQ ID NO: 525 - DNA, 30 bp, Artificial Sequence
  Description of Artificial Sequence Synthetic primer
ttcccattattatagagatgattgttgaat

SEQ ID NO: 526 - DNA, 26 bp, Artificial Sequence
  Description of Artificial Sequence Synthetic primer
ccagatactagagtgtctgtgtaatc

SEQ ID NO: 527 - DNA, 27 bp, Artificial Sequence
  Description of Artificial Sequence Synthetic primer
cattggcatggggaaatataaacttgt

SEQ ID NO: 528 - DNA, 28 bp, Artificial Sequence
  Description of Artificial Sequence Synthetic primer
aatagggttcagcaaatcttctaatcca

SEQ ID NO: 529 - DNA, 22 bp, Artificial Sequence
  Description of Artificial Sequence Synthetic primer
tggctttgaatctttggccagt

SEQ ID NO: 530 - DNA, 25 bp, Artificial Sequence
  Description of Artificial Sequence Synthetic primer
acataagagagaaggtttgactgcc

SEQ ID NO: 531 - DNA, 29 bp, Artificial Sequence
  Description of Artificial Sequence Synthetic primer
ttttggattacaggtgcttatgaatcaac

SEQ ID NO: 532 - DNA, 27 bp, Artificial Sequence
  Description of Artificial Sequence Synthetic primer
tctttgacggcaatattacgaaatcct

SEQ ID NO: 533 - DNA, 28 bp, Artificial Sequence
  Description of Artificial Sequence Synthetic primer
gtcatataggaagtagaggaaagtattc

SEQ ID NO: 534 - DNA, 28 bp, Artificial Sequence
  Description of Artificial Sequence Synthetic primer
ttaacaggaaatttctaaatgtgacatg

SEQ ID NO: 535 - DNA, 25 bp, Artificial Sequence
  Description of Artificial Sequence Synthetic primer
tctgtcaccaggtacagtaagtagg

SEQ ID NO: 536 - DNA, 27 bp, Artificial Sequence
  Description of Artificial Sequence Synthetic primer
aaaggaatagttgcatgtacagagtca

SEQ ID NO: 537 - DNA, 21 bp, Artificial Sequence
  Description of Artificial Sequence Synthetic primer
cgagatcgtgctgttccactc

SEQ ID NO: 538 - DNA, 29 bp, Artificial Sequence
  Description of Artificial Sequence Synthetic primer
gtgtaagattgagaaatctccaaggatct

SEQ ID NO: 539 - DNA, 29 bp, Artificial Sequence
  Description of Artificial Sequence Synthetic primer
acacaaagagaatctagtgattacagtgt

SEQ ID NO: 540 - DNA, 26 bp, Artificial Sequence
  Description of Artificial Sequence Synthetic primer
accaaggcacaagatcaaaatcattc

SEQ ID NO: 541 - DNA, 29 bp, Artificial Sequence
  Description of Artificial Sequence Synthetic primer
tgggaagtaataaaagatcaccttcagaa

SEQ ID NO: 542 - DNA, 28 bp, Artificial Sequence
  Description of Artificial Sequence Synthetic primer
tgaaaggattccactgaaagttttctga

SEQ ID NO: 543 - DNA, 24 bp, Artificial Sequence
  Description of Artificial Sequence Synthetic primer
tttgatgaggtgaagtccattgct

SEQ ID NO: 544 - DNA, 22 bp, Artificial Sequence
  Description of Artificial Sequence Synthetic primer
gtctctctttgctgtgccatcc

SEQ ID NO: 545 - DNA, 27 bp, Artificial Sequence
  Description of Artificial Sequence Synthetic primer
tcttccttattttgcctatgagggtac

SEQ ID NO: 546 - DNA, 24 bp, Artificial Sequence
  Description of Artificial Sequence Synthetic primer
ttgaagccatacctgttttcccaa

SEQ ID NO: 547 - DNA, 23 bp, Artificial Sequence
  Description of Artificial Sequence Synthetic oligonucleotide
tcagacgtgtgctcttccgatct

SEQ ID NO: 548 - DNA, 60 bp, Artificial Sequence
  Description of Artificial Sequence Synthetic primer
  modified_base (41)..(60): a, c, t, g, unknown or other
tcagacgtgtgctcttccgatctagcaggatcggtatggcnnnnnnnnnnnnnnnnnnnn

SEQ ID NO: 549 - DNA, 43 bp, Artificial Sequence
  Description of Artificial Sequence Synthetic primer
  modified_base (24)..(43): a, c, t, g, unknown or other
tcagacgtgtgctcttccgatctnnnnnnnnnnnnnnnnnnnn

SEQ ID NO: 550 - DNA, 75 bp, Artificial Sequence
  Description of Artificial Sequence Synthetic oligonucleotide
aatgatacggcgaccaccgagatctacactctttccctacacgacgctcttccgatctagcaggatcggtatggc

SEQ ID NO: 551 - DNA, 66 bp, Artificial Sequence
  Description of Artificial Sequence Synthetic oligonucleotide
caagcagaagacggcatacgagatttctgaatgtgactggagttcagacgtgtgctcttccgatct

SEQ ID NO: 552 - DNA, 66 bp, Artificial Sequence
  Description of Artificial Sequence Synthetic oligonucleotide
caagcagaagacggcatacgagatacgaattcgtgactggagttcagacgtgtgctcttccgatct

SEQ ID NO: 553 - DNA, 23 bp, Artificial Sequence
  Description of Artificial Sequence Synthetic oligonucleotide
tcagacgtgtgctcttccgatct

SEQ ID NO: 554 - DNA, 23 bp, Artificial Sequence
  Description of Artificial Sequence Synthetic oligonucleotide
tcagacgtgtgctcttccgatct

SEQ ID NO: 555 - DNA, 17 bp, Artificial Sequence
  Description of Artificial Sequence Synthetic oligonucleotide
agcaggatcggtatggc

SEQ ID NO: 556 - DNA, 24 bp, Artificial Sequence
  Description of Artificial Sequence Synthetic oligonucleotide
  Description of Combined DNA/RNA Molecule Synthetic oligonucleotide
agtcuuuuggttttttttttttgg

SEQ ID NO: 557 - DNA, 24 bp, Artificial Sequence
  Description of Artificial Sequence Synthetic oligonucleotide
  Description of Combined DNA/RNA Molecule Synthetic oligonucleotide
agtcuuuuccaaaaaaaaaaaacc

SEQ ID NO: 558 - DNA, 21 bp, Artificial Sequence
  Description of Artificial Sequence Synthetic oligonucleotide
  Description of Combined DNA/RNA Molecule Synthetic oligonucleotide
agtcuuuuggtttttttttgg

SEQ ID NO: 559 - DNA, 21 bp, Artificial Sequence
  Description of Artificial Sequence Synthetic oligonucleotide
  Description of Combined DNA/RNA Molecule Synthetic oligonucleotide
agtcuuuuccaaaaaaaaacc

SEQ ID NO: 560 - DNA, 46 bp, Artificial Sequence
  Description of Artificial Sequence Synthetic oligonucleotide
  Description of Combined DNA/RNA Molecule Synthetic oligonucleotide
agtcuuuugcggctttttttttgcgcgcgcgctttttttttggcgg

SEQ ID NO: 561 - DNA, 46 bp, Artificial Sequence
  Description of Artificial Sequence Synthetic oligonucleotide
  Description of Combined DNA/RNA Molecule Synthetic oligonucleotide
agtcuuuuccgccaaaaaaaaagcgcgcgcgcaaaaaaaaagccgc

SEQ ID NO: 562 - DNA, 46 bp, Artificial Sequence; identical to SEQ ID NO: 560
  Description of Artificial Sequence Synthetic oligonucleotide
  Description of Combined DNA/RNA Molecule Synthetic oligonucleotide

SEQ ID NO: 563 - DNA, 46 bp, Artificial Sequence; identical to SEQ ID NO: 561
  Description of Artificial Sequence Synthetic oligonucleotide
  Description of Combined DNA/RNA Molecule Synthetic oligonucleotide

SEQ ID NO: 564 - DNA, 33 bp, Artificial Sequence
  Description of Artificial Sequence Synthetic oligonucleotide
  Description of Combined DNA/RNA Molecule Synthetic oligonucleotide
agtcuuuugcgtttttttttgtttttttttggc

SEQ ID NO: 565 - DNA, 33 bp, Artificial Sequence
  Description of Artificial Sequence Synthetic oligonucleotide
  Description of Combined DNA/RNA Molecule Synthetic oligonucleotide
agtcuuuugccaaaaaaaaacaaaaaaaaacgc

SEQ ID NO: 566 - DNA, 27 bp, Artificial Sequence
  Description of Artificial Sequence Synthetic oligonucleotide
  Description of Combined DNA/RNA Molecule Synthetic oligonucleotide
agtcuuuugcgttttttgttttttggc

SEQ ID NO: 567 - DNA, 27 bp, Artificial Sequence
  Description of Artificial Sequence Synthetic oligonucleotide
  Description of Combined DNA/RNA Molecule Synthetic oligonucleotide
agtcuuuugccaaaaaacaaaaaacgc

SEQ ID NO: 568 - DNA, 24 bp, Artificial Sequence
  Description of Artificial Sequence Synthetic oligonucleotide
  Description of Combined DNA/RNA Molecule Synthetic oligonucleotide
agtcuuuuggttttttttttttgg

SEQ ID NO: 569 - DNA, 24 bp, Artificial Sequence
  Description of Artificial Sequence Synthetic oligonucleotide
  Description of Combined DNA/RNA Molecule Synthetic oligonucleotide
agtcuuuuccaaaaaaaaaaaacc

SEQ ID NO: 570 - DNA, 45 bp, Artificial Sequence
  Description of Artificial Sequence Synthetic oligonucleotide
  Description of Combined DNA/RNA Molecule Synthetic oligonucleotide
agtcggggcggctttttttttgcgcgcgcgctttttttttggcgg

SEQ ID NO: 571 - DNA, 45 bp, Artificial Sequence
  Description of Artificial Sequence Synthetic oligonucleotide
  Description of Combined DNA/RNA Molecule Synthetic oligonucleotide
agtcgggccgccaaaaaaaaagcgcgcgcgcaaaaaaaaagccgc

SEQ ID NO: 572 - DNA, 32 bp, Artificial Sequence
  Description of Artificial Sequence Synthetic oligonucleotide
  Description of Combined DNA/RNA Molecule Synthetic oligonucleotide
agtcggggcgtttttttttgtttttttttggc

SEQ ID NO: 573 - DNA, 32 bp, Artificial Sequence
  Description of Artificial Sequence Synthetic oligonucleotide
  Description of Combined DNA/RNA Molecule Synthetic oligonucleotide
agtcggggccaaaaaaaaacaaaaaaaaacgc

SEQ ID NO: 574 - DNA, 26 bp, Artificial Sequence
  Description of Artificial Sequence Synthetic oligonucleotide
  Description of Combined DNA/RNA Molecule Synthetic oligonucleotide
agtcggggcgttttttgttttttggc

SEQ ID NO: 575 - DNA, 26 bp, Artificial Sequence
  Description of Artificial Sequence Synthetic oligonucleotide
  Description of Combined DNA/RNA Molecule Synthetic oligonucleotide
agtcggggccaaaaaacaaaaaacgc

SEQ ID NO: 576 - DNA, 23 bp, Artificial Sequence
  Description of Artificial Sequence Synthetic oligonucleotide
  Description of Combined DNA/RNA Molecule Synthetic oligonucleotide
agtcgggcgttttttttttttcg

SEQ ID NO: 577 - DNA, 23 bp, Artificial Sequence
  Description of Artificial Sequence Synthetic oligonucleotide
  Description of Combined DNA/RNA Molecule Synthetic oligonucleotide
agtcgggcgaaaaaaaaaaaacg